(19) United States
(12) Patent Application Publication
(10) Pub. No.: US 2026/0215153 A1
(43) Pub. Date: Jul. 23, 2026

(54) PLURALITY OF HOST MATERIALS AND ORGANIC ELECTROLUMINESCENT DEVICE COMPRISING THE SAME

(71) Applicant: DuPont Specialty Materials Korea Ltd., Chungcheongnam-do (KR)

(72) Inventors: So-Young JUNG, Gyeonggi-do (KR); Su-Hyun LEE, Gyeonggi-do (KR); Mi-Ja LEE, Gyeonggi-do (KR); Doo-Hyeon MOON, Gyeonggi-do (KR); Sang-Hee CHO, Gyeonggi-do (KR)

(21) Appl. No.: 19/570,287

(22) Filed: Mar. 18, 2026

Related U.S. Application Data
(63) Continuation of application No. 17/119,922, filed on Dec. 11, 2020, now abandoned.

(30) Foreign Application Priority Data
Dec. 26, 2019 (KR) 10-2019-0175302

(51) Int. Cl. H10K 85/60; C07D 263/52; C07D 403/10; C07D 413/04; C07D 413/14

(52) U.S. Cl. CPC H10K 85/6565; C07D 263/52; C07D 403/10; C07D 413/04; C07D 413/14; H10K 85/6572

(57) ABSTRACT

The present disclosure relates to a plurality of host materials comprising a first host material comprising a compound represented by formula 1, and a second host material comprising a compound represented by formula 2, and an organic electroluminescent device comprising the same. By comprising a specific combination of compounds as host materials, it is possible to provide an organic electroluminescent device having lower driving voltage, higher luminous efficiency, higher power efficiency, and/or better lifetime properties, compared to conventional organic electroluminescent devices.

### CLAIM OF BENEFIT OF PRIOR APPLICATION

This application is a continuation of U.S. patent application Ser. No. 17/119,922, filed Dec. 11, 2020, which is incorporated by reference herein in its entirety.

### TECHNICAL FIELD

The present disclosure relates to a plurality of host materials and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. An OLED having high luminous efficiency and/or long lifetime characteristics is required for long-time use and high resolution of a display.

In order to enhance luminous efficiency, driving voltage, and/or lifetime properties, various materials or concepts for an organic layer of an organic electroluminescent device have been proposed. However, they were not satisfactory in practical use.

Korean Laid-open Patent Application No. 2015-0124902 discloses a plurality of host materials using a compound of a carbazole derivative, and Korean Laid-open Patent Application Nos. 2017-0022865 and 2018-0099487 disclose a host compound having a phenanthrooxazole-type and/or phenanthrothiazole-type compound as a basic structure. However, these references do not specifically disclose the plurality of host materials of the present disclosure. In addition, there is a continuous need for developing a light-emitting material having improved performance such as low driving voltage, high luminous efficiency, high power efficiency, and/or excellent lifetime properties.

### DISCLOSURE OF INVENTION

### Technical Problem

The objective of the present disclosure is to provide an organic electroluminescent device having low driving voltage, high luminous efficiency, high power efficiency and/or excellent lifetime properties by comprising a plurality of host materials including a specific combination of compounds.

### Solution to Problem

The present inventors found that the above objective can be achieved by a plurality of host materials comprising a first host material comprising a compound represented by the following formula 1, and a second host material comprising a compound represented by the following formula 2:

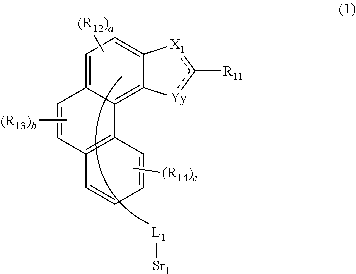

wherein
X₁ and Y₁ each independently represent —N═, —NR₁₅—, —O—, or —S—, with the proviso that one of X₁ and Y₁ is —N═, and the other of X₁ and Y₁ is —NR₁₅—, —O—, or —S—;
L₁ represents a single bond, or a substituted or unsubstituted (C6-C30) arylene;
Ar₁ represents a substituted or unsubstituted (C6-C30) aryl, a substituted or unsubstituted (3- to 30-membered) heteroaryl, a substituted or unsubstituted mono- or di-(C1-C30)alkylamino, a substituted or unsubstituted mono- or di-(C6-C30) arylamino, a substituted or unsubstituted (C1-C30)alkyl(C6-C30) arylamino, or a substituted or unsubstituted (C6-C30) aryl(3- to 30-membered) heteroarylamino;
R₁₁ represents a substituted or unsubstituted (C6-C30) aryl, or a substituted or unsubstituted (3- to 30-membered) heteroaryl;
R₁₂ to R₁₅ each independently represent hydrogen, deuterium, a halogen, a cyano, a substituted or unsubstituted (C1-C30)alkyl, a substituted or unsubstituted (C6-C30) aryl, a substituted or unsubstituted (3- to 30-membered) heteroaryl, a substituted or unsubstituted (C3-C30)cycloalkyl, a substituted or unsubstituted (C1-C30)alkoxy, a substituted or unsubstituted tri (C1-C30)alkylsilyl, a substituted or unsubstituted di(C1-C30)alkyl(C6-C30) arylsilyl, a substituted or unsubstituted (C1-C30)alkyldi (C6-C30) arylsilyl, a substituted or unsubstituted tri (C6-C30) arylsilyl, a substituted or unsubstituted mono- or di-(C1-C30)alkylamino, a substituted or unsubstituted mono- or di-(C6-C30) arylamino, or a substituted or unsubstituted (C1-C30)alkyl(C6-C30) arylamino; or may be linked to an adjacent substituent to form a ring(s);
a and b each independently represent 1 or 2, and c is an integer of 1 to 3, where if a to c are an integer of 2 or more, each of R₁₂ to each of R₁₄ may be the same or different;

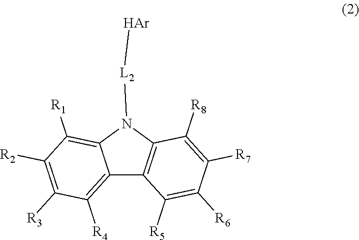

wherein
HAr represents a substituted or unsubstituted (3- to 30-membered) heteroaryl containing a nitrogen atom(s);
L₂ represents a single bond, or a substituted or unsubstituted (C6-C30) arylene; and
R₁ to R₈ each independently represent hydrogen, deuterium, or a (C6-C30) aryl unsubstituted or substituted with deuterium.

### Advantageous Effects of Invention

By comprising the plurality of host materials according to the present disclosure, an organic electroluminescent device having lower driving voltage, higher luminous efficiency, higher power efficiency and/or better lifetime properties compared to conventional organic electroluminescent devices can be provided, and it is possible to produce a display device or a lighting device using the same.

### MODE FOR THE INVENTION

Hereinafter, the present disclosure will be described in detail. However, the following description is intended to explain the invention and is not meant in any way to restrict the scope of the present disclosure.

The term “organic electroluminescent material” in the present disclosure means a material that may be used in an organic electroluminescent device, and may comprise at least one compound. The organic electroluminescent material may be comprised in any layer constituting an organic electroluminescent device as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material of a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or different layers, and may be mixture-evaporated or co-evaporated, or may be individually evaporated.

The term “a plurality of host materials” in the present disclosure means an organic electroluminescent material of a combination of two or more host materials. It may mean both a material before being comprised in an organic electroluminescent device (for example, before vapor deposition) and a material after being comprised in an organic electroluminescent device (for example, after vapor deposition). The plurality of host materials of the present disclosure may be comprised in any of the light-emitting layers constituting the organic electroluminescent device. The at least two compounds comprised in the plurality of host materials may be comprised together in one light-emitting layer or may respectively be comprised in different light-emitting layers. When the at least two host materials are comprised in one layer, these may be mixture-evaporated to form a layer, or separately co-evaporated at the same time to form a layer, for example.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, iso-propyl, n-butyl, iso-butyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered) heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30) aryl(ene)” is meant to be a monocyclic or fused ring radical derived from an aromatic hydrocarbon having 6 to 30 ring backbone carbon atoms, in which the number of the ring backbone carbon atoms is preferably 6 to 25, and more preferably 6 to 18. The above aryl(ene) may be partially saturated, and may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, etc. More specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenylyl, 4″-t-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, etc.

The term “(3- to 30-membered) heteroaryl or (3- to 50-membered) heteroaryl” is meant to be an aryl having 3 to 30 or 3 to 50 ring backbone atoms, and including at least one, preferably 1 to 4 heteroatoms selected from the group consisting of B, N, O, S, Si, and P. The above heteroaryl may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, naphthobenzofuranyl, naphthobenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, and dihydroacridinyl. More specifically, the above heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl) pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent. The substituents of the substituted alkyl, the substituted aryl, the substituted arylene, the substituted heteroaryl, the substituted cycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, the substituted alkylarylamino, and the substituted arylheteroarylamino in the formulas of the present disclosure, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered) heterocycloalkyl; a (C6-C30) aryloxy; a (C6-C30) arylthio; a (3- to 50-membered) heteroaryl unsubstituted or substituted with at least one of a (C1-C30)alkyl(s), a (C6-C30) aryl(s), and a di(C6-C30) arylamino(s); a (C6-C30) aryl unsubstituted or substituted with at least one of deuterium, a cyano(s), a (C1-C30)alkyl(s), a (3- to 50-membered) heteroaryl(s), a di(C6-C30) arylamino(s), and a tri (C6-C30) arylsilyl(s); a tri (C1-C30)alkylsilyl; a tri (C6-C30) arylsilyl; a di(C1-C30)alkyl(C6-C30) arylsilyl; a (C1-C30)alkyldi(C6-C30) arylsilyl; an amino; a mono- or di-(C1-C30)alkylamino; a mono- or di-(C6-C30) arylamino; a (C1-C30)alkyl(C6-C30) arylamino; a (C1-C30)alkylcarbonyl; a (C1—C30)alkoxycarbonyl; a (C6-C30) arylcarbonyl; a di(C6-C30) arylboronyl; a di(C1-C30)alkylboronyl; a (C1-C30)alkyl(C6-C30) arylboronyl; a (C6-C30) aryl(C1-C30)alkyl; and a (C1-C30)alkyl(C6-C30) aryl, and the substituents may be substituted with deuterium instead of hydrogen in an optional position. According to one embodiment of the present disclosure, the substituents each independently are at least one selected from the group consisting of deuterium, a (C1-C6)alkyl, a (C6-C25) aryl unsubstituted or substituted with deuterium, a (5- to 15-membered) heteroaryl, and a di(C6-C12) arylamino. Specifically, the substituents each independently may be at least one selected from the group consisting of deuterium, a methyl, a phenyl, a naphthyl, a biphenyl, a terphenyl, a fluorenyl, a spirobifluorenyl, a phenanthrenyl, a phenyl substituted with deuterium, a naphthylphenyl, a naphthyl substituted with deuterium, a phenylnaphthyl, a dibenzofuranyl, a dibenzothiophenyl, a carbazolyl, and a diphenylamino.

In the formulas of the present disclosure, if a substituent is linked to an adjacent substituent to form a ring, the ring may be a substituted or unsubstituted, mono- or polycyclic, (3- to 30-membered) alicyclic or aromatic ring, or the combination thereof, which two or more adjacent substituents are linked to form. In addition, the formed ring may contain at least one heteroatom selected from B, N, O, S, Si, and P, preferably at least one heteroatom selected from N, O, and S. According to one embodiment of the present disclosure, the number of the ring backbone atoms is 5 to 20. According to another embodiment of the present disclosure, the number of the ring backbone atoms is 5 to 15.

In the formulas of the present disclosure, heteroaryl may, each independently, contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30) aryl, a substituted or unsubstituted (5- to 30-membered) heteroaryl, a substituted or unsubstituted (C3-C30)cycloalkyl, a substituted or unsubstituted (C1-C30)alkoxy, a substituted or unsubstituted tri (C1-C30)alkylsilyl, a substituted or unsubstituted di(C1-C30)alkyl(C6-C30) arylsilyl, a substituted or unsubstituted (C1-C30)alkyldi (C6-C30) arylsilyl, a substituted or unsubstituted tri (C6-C30) arylsilyl, a substituted or unsubstituted mono- or di-(C1-C30)alkylamino, a substituted or unsubstituted mono- or di-(C6-C30) arylamino, and a substituted or unsubstituted (C1-C30)alkyl(C6-C30) arylamino.

The plurality of host materials according to one embodiment of the present disclosure comprises a first host material comprising the compound represented by formula 1 and a second host material comprising the compound represented by formula 2, and may be comprised in a light-emitting layer of an organic electroluminescent device according to one embodiment of the present disclosure.

Hereinafter, the compound represented by formula 1 will be described in more detail.

In formula 1, L₁ may be linked to the mother nucleus, which is the phenanthrene ring, at any position of the mother nucleus. Preferably, L₁ may be linked to the benzene ring of which R₁₄ is bonded, and more preferably, L₁ may be linked to the benzene ring of which R₁₄ is bonded at the 2- or 3-position.

According to one embodiment of the present disclosure, the compound represented by formula 1 may be represented by at least one of the following formulas 1-1 and 1-2:

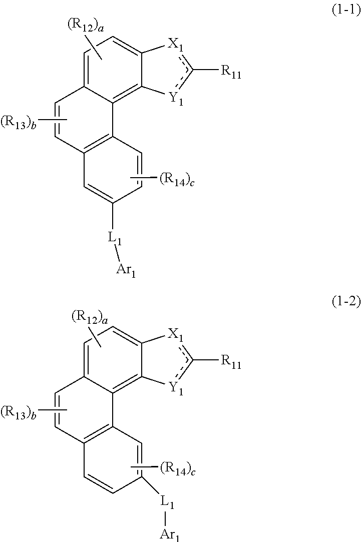

wherein X₁, Y₁, Ar₁, L₁, R₁₁ to R₁₄, and a to c are as defined in formula 1.

In formula 1, X₁ and Y₁ each independently represent —N═, —NR₁₅—, —O—, or —S—, with the proviso that one of X₁ and Y₁ is —N═, and the other of X₁ and Y₁ is —NR₁₅—, —O—, or —S—. According to one embodiment of the present disclosure, when X₁ is —N═, Y₁ is —NR₁₅—, —O—, or —S—, and when Y₁ is —N═, X₁ is —NR₁₅—, —O—, or —S—. According to another embodiment of the present disclosure, when X₁ is —N═, Y₁ is —O— or —S—, and when Y₁ is —N═, X₁ is —NR₁₅—, —O—, or —S—. Herein, R₁₅ represents hydrogen, deuterium, a halogen, a cyano, a substituted or unsubstituted (C1-C30)alkyl, a substituted or unsubstituted (C6-C30) aryl, a substituted or unsubstituted (3- to 30-membered) heteroaryl, a substituted or unsubstituted (C3-C30)cycloalkyl, a substituted or unsubstituted (C1-C30)alkoxy, a substituted or unsubstituted tri (C1-C30)alkylsilyl, a substituted or unsubstituted di(C1-C30)alkyl(C6-C30) arylsilyl, a substituted or unsubstituted (C1-C30)alkyldi (C6-C30) arylsilyl, a substituted or unsubstituted tri (C6-C30) arylsilyl, a substituted or unsubstituted mono- or di-(C1-C30)alkylamino, a substituted or unsubstituted mono- or di-(C6-C30) arylamino, or a substituted or unsubstituted (C1-C30)alkyl(C6-C30) arylamino; or may be linked to an adjacent substituent to form a ring(s). According to one embodiment of the present disclosure, R₁₅ may represent hydrogen, deuterium, a halogen, a cyano, a substituted or unsubstituted (C1-C30)alkyl, a substituted or unsubstituted (C6-C30) aryl, or a substituted or unsubstituted (3- to 30-membered) heteroaryl. According to another embodiment of the present disclosure, R₁₅ may represent hydrogen, deuterium, a halogen, a cyano, a substituted or unsubstituted (C1-C10)alkyl, or a substituted or unsubstituted (C6-C25) aryl. According to still another embodiment of the present disclosure, R₁₅ may represent hydrogen or a substituted or unsubstituted (C6-C18) aryl.

In formula 1, L₁ represents a single bond, or a substituted or unsubstituted (C6-C30) arylene. According to one embodiment of the present disclosure, L₁ represents a single bond, or a substituted or unsubstituted (C6-C25) arylene. According to another embodiment of the present disclosure, L₁ represents a single bond, or a substituted or unsubstituted (C6-C18) arylene. Specifically, L₁ may represent a single bond, a substituted or unsubstituted phenylene, a substituted or unsubstituted naphthylene, a substituted or unsubstituted biphenylene, etc.

In formula 1, Ar₁ represents a substituted or unsubstituted (C6-C30) aryl, a substituted or unsubstituted (3- to 30-membered) heteroaryl, a substituted or unsubstituted mono- or di-(C1-C30)alkylamino, a substituted or unsubstituted mono- or di-(C6-C30) arylamino, a substituted or unsubstituted (C1-C30)alkyl(C6-C30) arylamino, or a substituted or unsubstituted (C6-C30) aryl(3- to 30-membered) heteroarylamino. According to one embodiment of the present disclosure, Ar₁ represents a substituted or unsubstituted (C6-C30) aryl, a substituted or unsubstituted (5- to 30-membered) heteroaryl, a substituted or unsubstituted di(C6-C25) arylamino, or a substituted or unsubstituted (C6-C25) aryl(5- to 25-membered) heteroarylamino. According to another embodiment of the present disclosure, Ar₁ represents a substituted or unsubstituted (C6-C25) aryl, a substituted or unsubstituted (5- to 25-membered) heteroaryl containing at least one nitrogen atom, a substituted or unsubstituted di(C6-C18) arylamino, or a substituted or unsubstituted (C6-C18) aryl(5- to 18-membered) heteroarylamino. Specifically, Ar₁ may represent a substituted or unsubstituted phenyl, a substituted or unsubstituted naphthyl, a substituted or unsubstituted biphenyl, a substituted or unsubstituted terphenyl, a substituted or unsubstituted phenanthrenyl, a substituted or unsubstituted benzophenanthrenyl, a substituted or unsubstituted fluoranthenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted spiro[cyclopentane-fluorene]yl, a substituted or unsubstituted spiro[dihydroindene-fluorene]yl, a substituted or unsubstituted spiro[fluorene-benzofluorene]yl, a substituted or unsubstituted pyrimidinyl, a substituted or unsubstituted triazinyl, a substituted or unsubstituted quinazolinyl, a substituted or unsubstituted quinoxalinyl, an amino substituted with at least one of a substituted or unsubstituted phenyl(s), a naphthyl(s), a biphenyl(s), a terphenyl(s), a phenanthrenyl(s), a substituted or unsubstituted fluorenyl(s), a substituted or unsubstituted carbazolyl(s), a dibenzofuranyl(s), and a dibenzothiophenyl(s), etc.

In formula 1, R₁₁ represents a substituted or unsubstituted (C6-C30) aryl, or a substituted or unsubstituted (3- to 30-membered) heteroaryl. According to one embodiment of the present disclosure, R₁₁ represents a substituted or unsubstituted (C6-C25) aryl, or a substituted or unsubstituted (5- to 25-membered) heteroaryl. According to another embodiment of the present disclosure, R₁₁ represents a substituted or unsubstituted (C6-C18) aryl, or a substituted or unsubstituted (5- to 18-membered) heteroaryl. Specifically, R₁₁ may represent a substituted or unsubstituted phenyl, a substituted or unsubstituted naphthyl, a substituted or unsubstituted biphenyl, a substituted or unsubstituted fluorenyl, a substituted or unsubstituted dibenzothiophenyl, a substituted or unsubstituted quinolinyl, a substituted or unsubstituted isoquinolinyl, etc.

In formula 1, R₁₂ to R₁₄ each independently represent hydrogen, deuterium, a halogen, a cyano, a substituted or unsubstituted (C1-C30)alkyl, a substituted or unsubstituted (C6-C30) aryl, a substituted or unsubstituted (3- to 30-membered) heteroaryl, a substituted or unsubstituted (C3-C30)cycloalkyl, a substituted or unsubstituted (C1-C30)alkoxy, a substituted or unsubstituted tri (C1-C30)alkylsilyl, a substituted or unsubstituted di(C1-C30)alkyl(C6-C30) arylsilyl, a substituted or unsubstituted (C1-C30)alkyldi (C6-C30) arylsilyl, a substituted or unsubstituted tri (C6-C30) arylsilyl, a substituted or unsubstituted mono- or di-(C1-C30)alkylamino, a substituted or unsubstituted mono- or di-(C6-C30) arylamino, or a substituted or unsubstituted (C1-C30)alkyl(C6-C30) arylamino; or may be linked to an adjacent substituent to form a ring(s). According to one embodiment of the present disclosure, R₁₂ to R₁₄ each independently represent hydrogen, deuterium, a halogen, a cyano, a substituted or unsubstituted (C1-C10)alkyl, a substituted or unsubstituted (C6-C25) aryl, or a substituted or unsubstituted (5- to 25-membered) heteroaryl. According to another embodiment of the present disclosure, R₁₂ to R₁₄ each independently represent hydrogen, deuterium, a halogen, a cyano, or a substituted or unsubstituted (C1-C10)alkyl.
a and b each independently represent 1 or 2, and c is an integer of 1 to 3, where if a to c are an integer of 2 or more, each of R₁₂ to each of R₁₄ may be the same or different.

The compound represented by formula 1 may be at least one selected from the following compounds, but is not limited thereto.

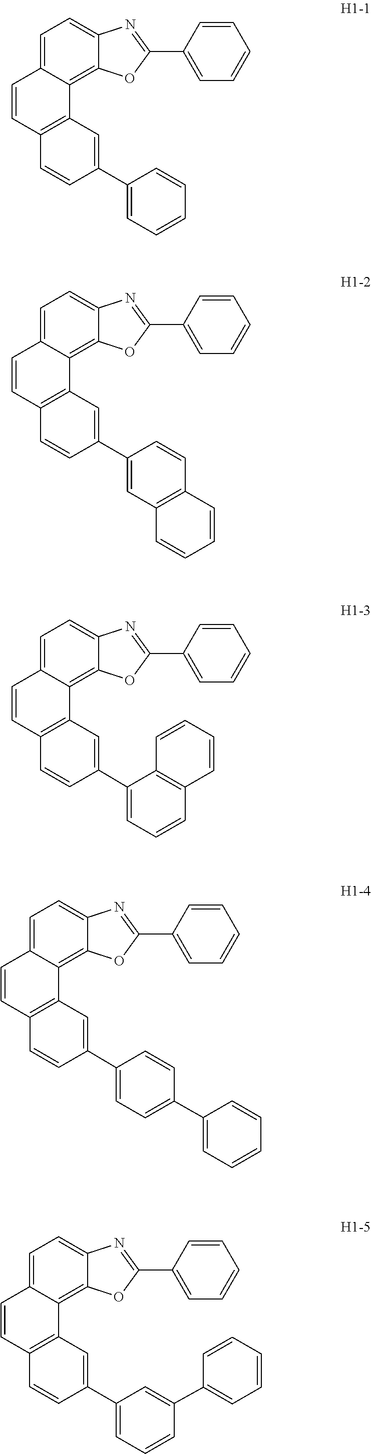 [SMILES: c1ccc(-c2ccc3ccc4ccc5nc(-c6ccccc6)oc5c4c3c2)cc1 ; c1ccc(-c2nc3ccc4ccc5ccc(-c6ccc7ccccc7c6)cc5c4c3o2)cc1 ; c1ccc(-c2nc3ccc4ccc5ccc(-c6cccc7ccccc67)cc5c4c3o2)cc1 ; c1ccc(-c2ccc(-c3ccc4ccc5ccc6nc(-c7ccccc7)oc6c5c4c3)cc2)cc1 ; c1ccc(-c2cccc(-c3ccc4ccc5ccc6nc(-c7ccccc7)oc6c5c4c3)c2)cc1]

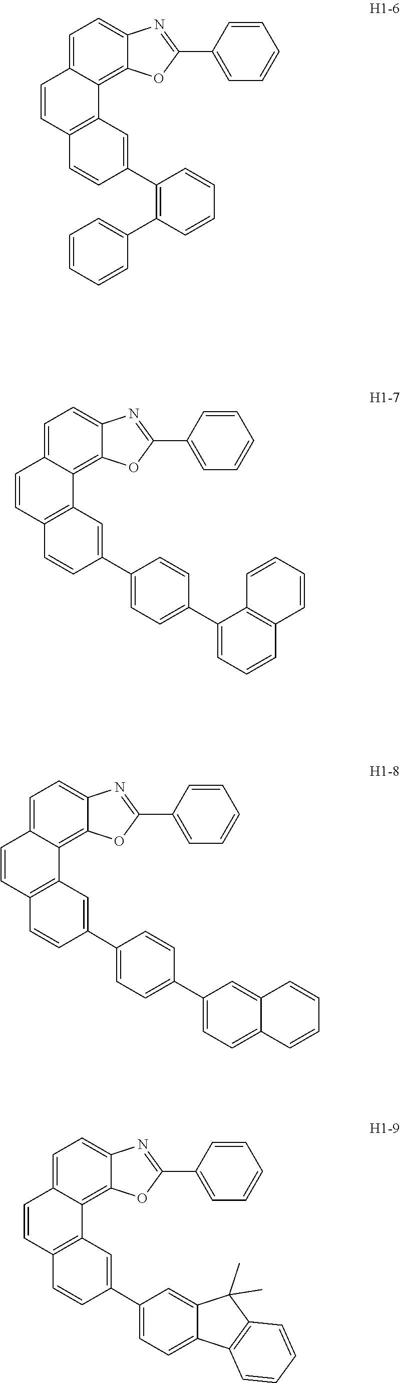 [SMILES: c1ccc(-c2nc3ccc4ccc5ccc(-c6ccccc6-c6ccccc6)cc5c4c3o2)cc1 ; c1ccc(-c2nc3ccc4ccc5ccc(-c6ccc(-c7cccc8ccccc78)cc6)cc5c4c3o2)cc1 ; c1ccc(-c2nc3ccc4ccc5ccc(-c6ccc(-c7ccc8ccccc8c7)cc6)cc5c4c3o2)cc1 ; CC1(C)c2ccccc2-c2ccc(-c3ccc4ccc5ccc6nc(-c7ccccc7)oc6c5c4c3)cc21]

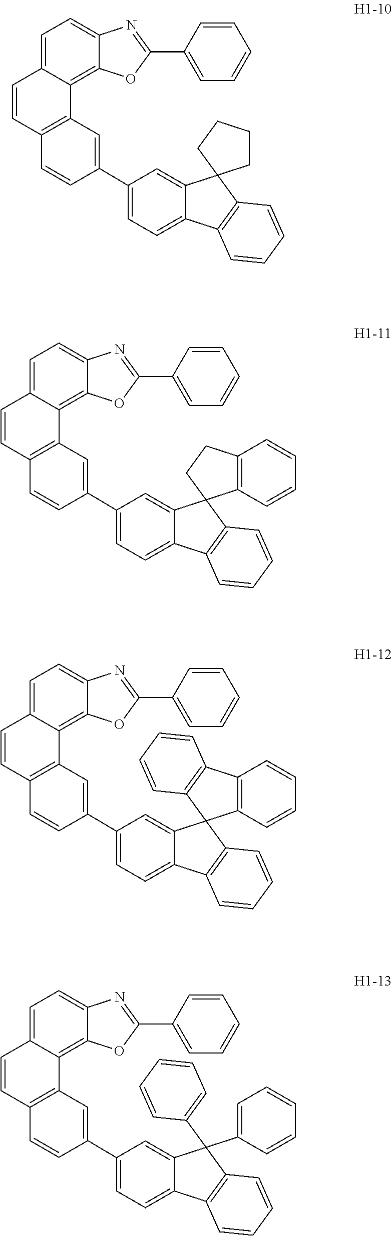 [SMILES: c1ccc(-c2nc3ccc4ccc5ccc(-c6ccc7c(c6)C6(CCCC6)c6ccccc6-7)cc5c4c3o2)cc1 ; c1ccc(-c2nc3ccc4ccc5ccc(-c6ccc7c(c6)C6(CCc8ccccc86)c6ccccc6-7)cc5c4c3o2)cc1 ; c1ccc(-c2nc3ccc4ccc5ccc(-c6ccc7c(c6)C6(c8ccccc8-c8ccccc86)c6ccccc6-7)cc5c4c3o2)cc1 ; c1ccc(-c2nc3ccc4ccc5ccc(-c6ccc7c(c6)C(c6ccccc6)(c6ccccc6)c6ccccc6-7)cc5c4c3o2)cc1]

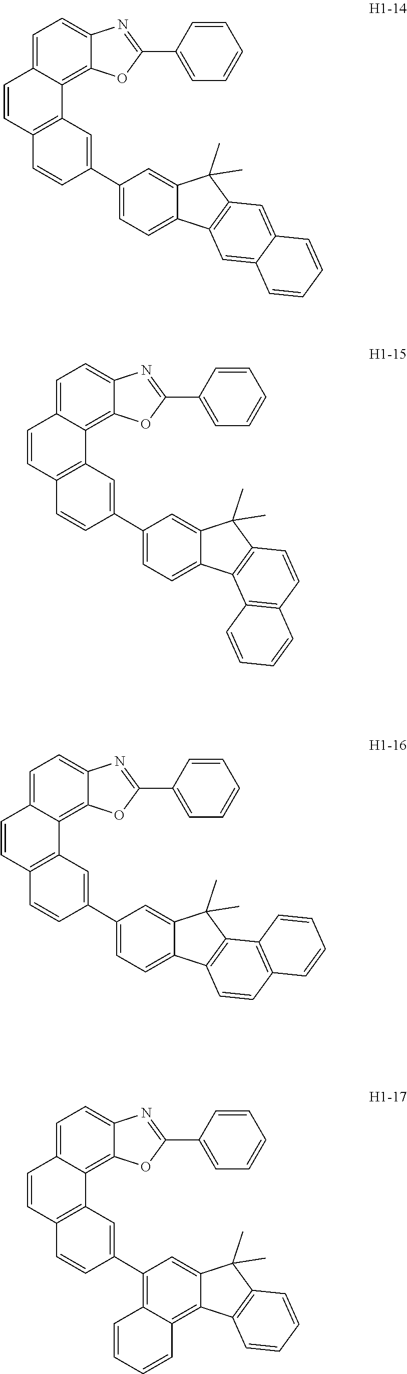 [SMILES: CC1(C)c2cc(-c3ccc4ccc5ccc6nc(-c7ccccc7)oc6c5c4c3)ccc2-c2cc3ccccc3cc21 ; CC1(C)c2cc(-c3ccc4ccc5ccc6nc(-c7ccccc7)oc6c5c4c3)ccc2-c2c1ccc1ccccc21 ; CC1(C)c2cc(-c3ccc4ccc5ccc6nc(-c7ccccc7)oc6c5c4c3)ccc2-c2ccc3ccccc3c21 ; CC1(C)c2ccccc2-c2c1cc(-c1ccc3ccc4ccc5nc(-c6ccccc6)oc5c4c3c1)c1ccccc21]

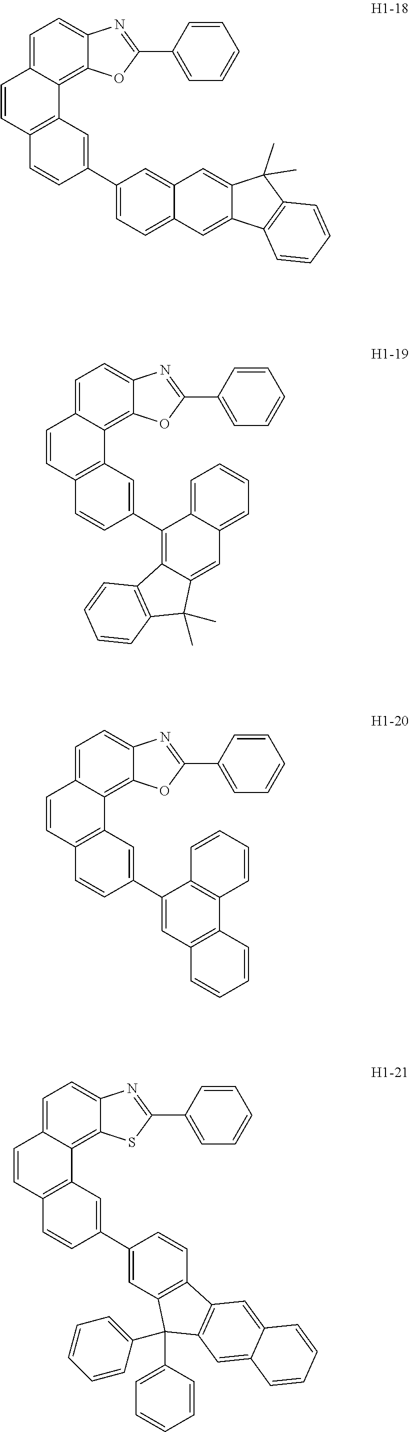 [SMILES: CC1(C)c2ccccc2-c2cc3ccc(-c4ccc5ccc6ccc7nc(-c8ccccc8)oc7c6c5c4)cc3cc21 ; CC1(C)c2ccccc2-c2c1cc1ccccc1c2-c1ccc2ccc3ccc4nc(-c5ccccc5)oc4c3c2c1 ; c1ccc(-c2nc3ccc4ccc5ccc(-c6cc7ccccc7c7ccccc67)cc5c4c3o2)cc1 ; c1ccc(-c2nc3ccc4ccc5ccc(-c6ccc7c(c6)C(c6ccccc6)(c6ccccc6)c6cc8ccccc8cc6-7)cc5c4c3s2)cc1]

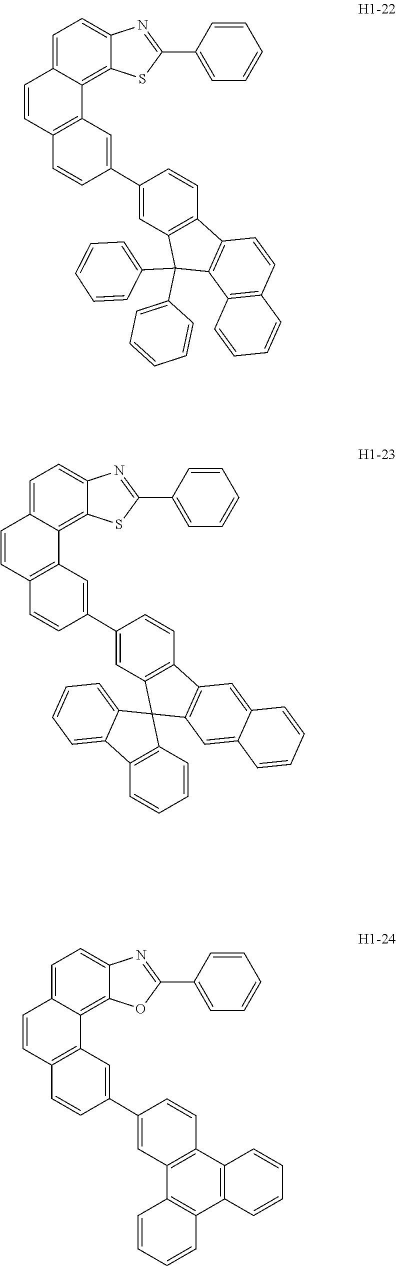 [SMILES: c1ccc(-c2nc3ccc4ccc5ccc(-c6ccc7c(c6)C(c6ccccc6)(c6ccccc6)c6c-7ccc7ccccc67)cc5c4c3s2)cc1 ; c1ccc(-c2nc3ccc4ccc5ccc(-c6ccc7c(c6)C6(c8ccccc8-c8ccccc86)c6cc8ccccc8cc6-7)cc5c4c3s2)cc1 ; c1ccc(-c2nc3ccc4ccc5ccc(-c6ccc7c8ccccc8c8ccccc8c7c6)cc5c4c3o2)cc1]

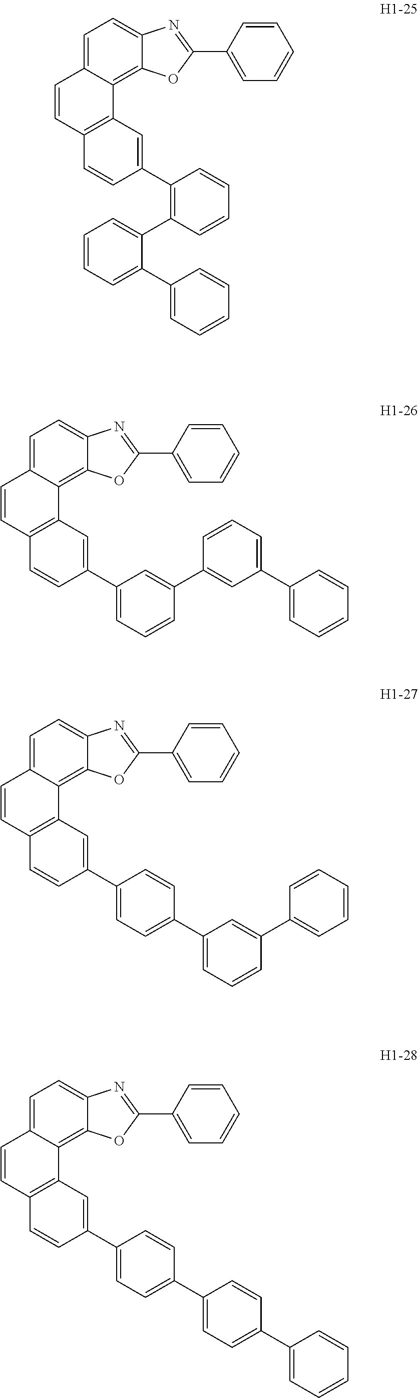 [SMILES: c1ccc(-c2nc3ccc4ccc5ccc(-c6ccccc6-c6ccccc6-c6ccccc6)cc5c4c3o2)cc1 ; c1ccc(-c2cccc(-c3cccc(-c4ccc5ccc6ccc7nc(-c8ccccc8)oc7c6c5c4)c3)c2)cc1 ; c1ccc(-c2cccc(-c3ccc(-c4ccc5ccc6ccc7nc(-c8ccccc8)oc7c6c5c4)cc3)c2)cc1 ; c1ccc(-c2ccc(-c3ccc(-c4ccc5ccc6ccc7nc(-c8ccccc8)oc7c6c5c4)cc3)cc2)cc1]

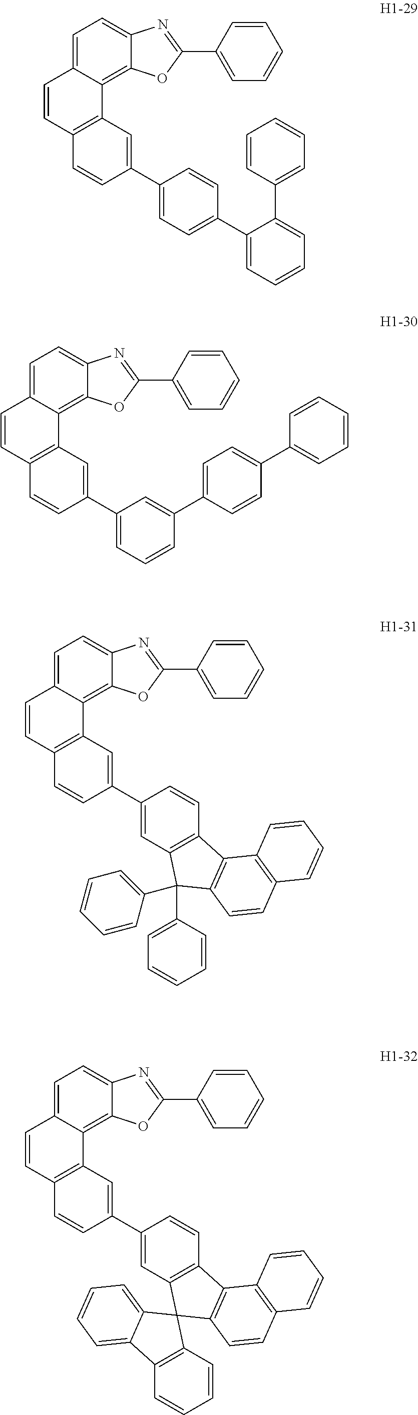 [SMILES: c1ccc(-c2nc3ccc4ccc5ccc(-c6ccc(-c7ccccc7-c7ccccc7)cc6)cc5c4c3o2)cc1 ; c1ccc(-c2ccc(-c3cccc(-c4ccc5ccc6ccc7nc(-c8ccccc8)oc7c6c5c4)c3)cc2)cc1 ; c1ccc(-c2nc3ccc4ccc5ccc(-c6ccc7c(c6)C(c6ccccc6)(c6ccccc6)c6ccc8ccccc8c6-7)cc5c4c3o2)cc1 ; c1ccc(-c2nc3ccc4ccc5ccc(-c6ccc7c(c6)C6(c8ccccc8-c8ccccc86)c6ccc8ccccc8c6-7)cc5c4c3o2)cc1]

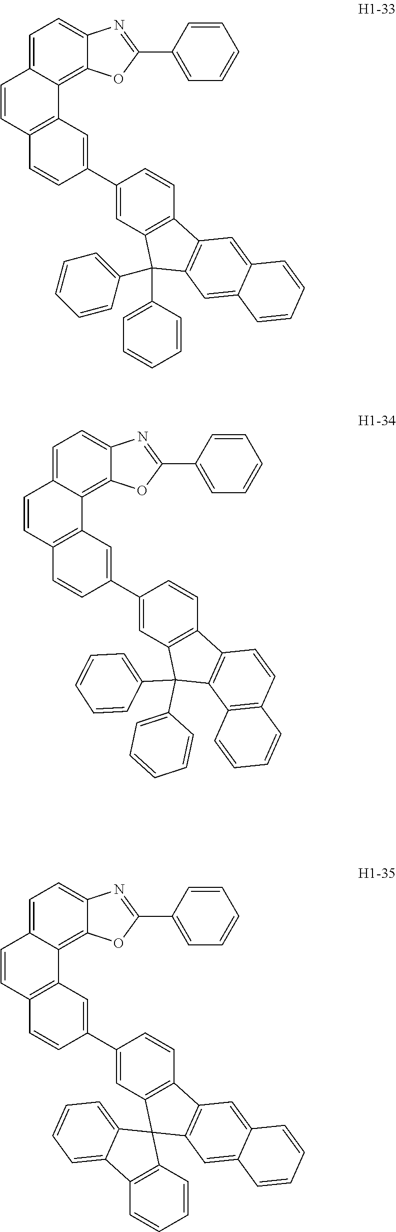 [SMILES: c1ccc(-c2nc3ccc4ccc5ccc(-c6ccc7c(c6)C(c6ccccc6)(c6ccccc6)c6cc8ccccc8cc6-7)cc5c4c3o2)cc1 ; c1ccc(-c2nc3ccc4ccc5ccc(-c6ccc7c(c6)C(c6ccccc6)(c6ccccc6)c6c-7ccc7ccccc67)cc5c4c3o2)cc1 ; c1ccc(-c2nc3ccc4ccc5ccc(-c6ccc7c(c6)C6(c8ccccc8-c8ccccc86)c6cc8ccccc8cc6-7)cc5c4c3o2)cc1]

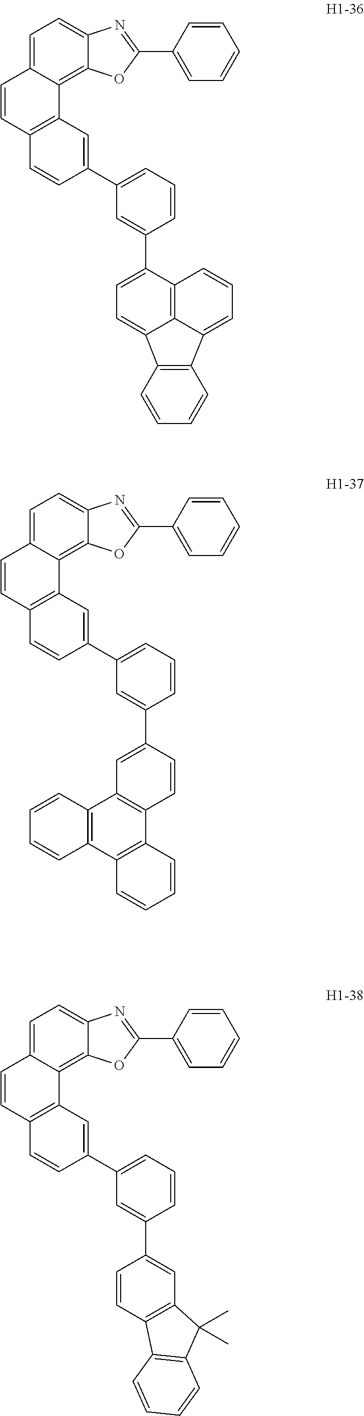 [SMILES: c1ccc(-c2nc3ccc4ccc5ccc(-c6cccc(-c7ccc8c9c(cccc79)-c7ccccc7-8)c6)cc5c4c3o2)cc1 ; c1ccc(-c2nc3ccc4ccc5ccc(-c6cccc(-c7ccc8c9ccccc9c9ccccc9c8c7)c6)cc5c4c3o2)cc1 ; CC1(C)c2ccccc2-c2ccc(-c3cccc(-c4ccc5ccc6ccc7nc(-c8ccccc8)oc7c6c5c4)c3)cc21]

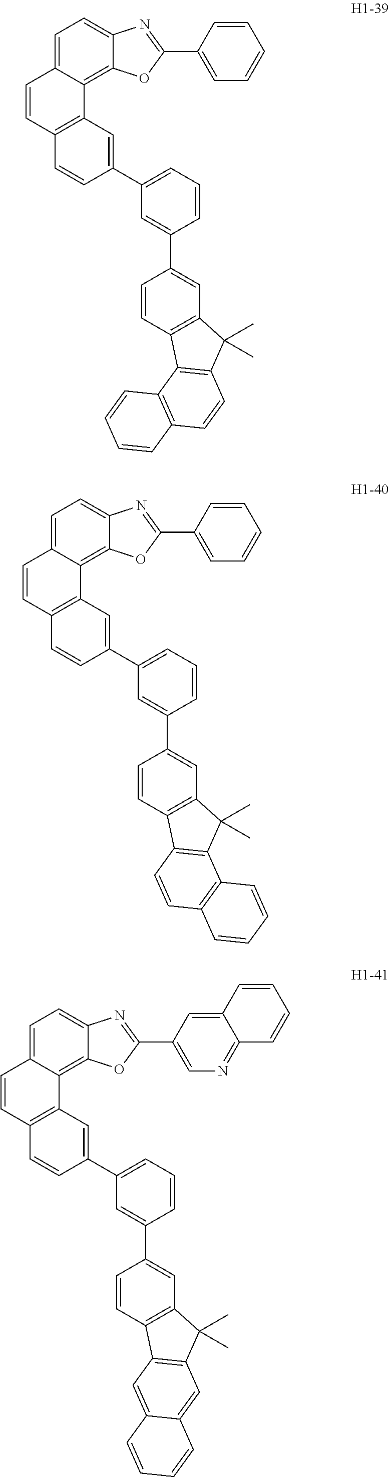

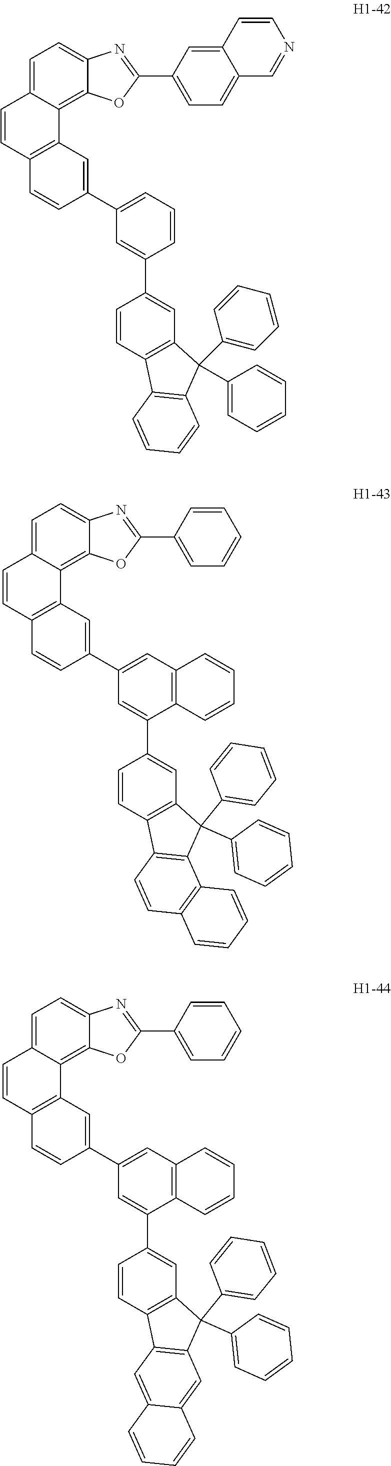 [SMILES: c1ccc(C2(c3ccccc3)c3ccccc3-c3ccc(-c4cccc(-c5ccc6ccc7ccc8nc(-c9ccc%10cnccc%10c9)oc8c7c6c5)c4)cc32)cc1 ; c1ccc(-c2nc3ccc4ccc5ccc(-c6cc(-c7ccc8c(c7)C(c7ccccc7)(c7ccccc7)c7c-8ccc8ccccc78)c7ccccc7c6)cc5c4c3o2)cc1 ; c1ccc(-c2nc3ccc4ccc5ccc(-c6cc(-c7ccc8c(c7)C(c7ccccc7)(c7ccccc7)c7cc9ccccc9cc7-8)c7ccccc7c6)cc5c4c3o2)cc1]

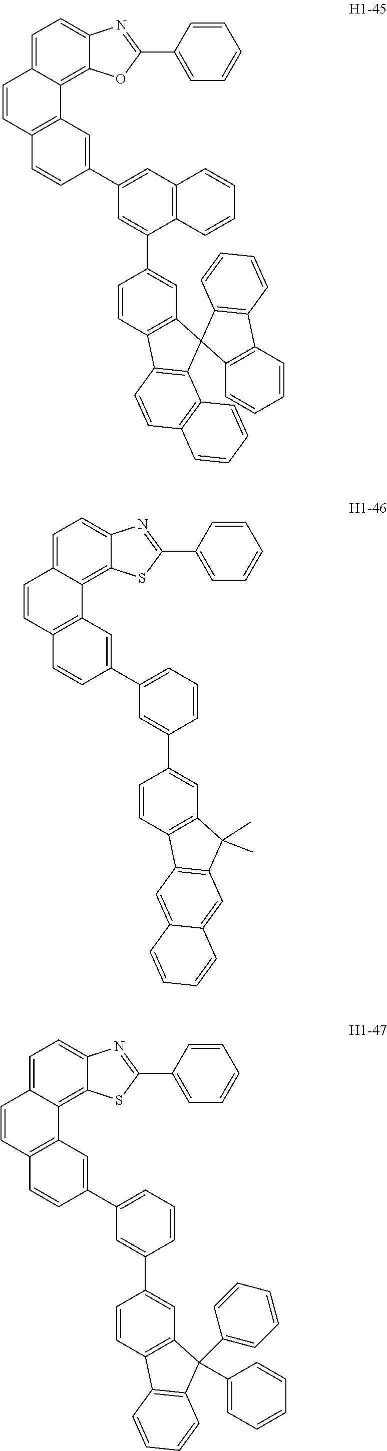 [SMILES: c1ccc(-c2nc3ccc4ccc5ccc(-c6cc(-c7ccc8c(c7)C7(c9ccccc9-c9ccccc97)c7c-8ccc8ccccc78)c7ccccc7c6)cc5c4c3o2)cc1 ; CC1(C)c2cc(-c3cccc(-c4ccc5ccc6ccc7nc(-c8ccccc8)sc7c6c5c4)c3)ccc2-c2cc3ccccc3cc21 ; c1ccc(-c2nc3ccc4ccc5ccc(-c6cccc(-c7ccc8c(c7)C(c7ccccc7)(c7ccccc7)c7ccccc7-8)c6)cc5c4c3s2)cc1]

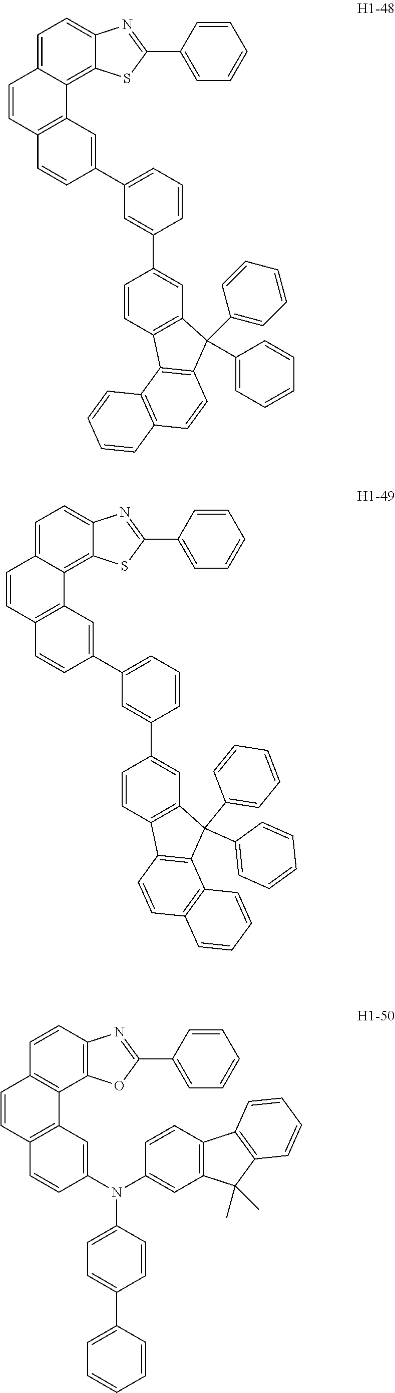 [SMILES: c1ccc(-c2nc3ccc4ccc5ccc(-c6cccc(-c7ccc8c(c7)C(c7ccccc7)(c7ccccc7)c7ccc9ccccc9c7-8)c6)cc5c4c3s2)cc1 ; c1ccc(-c2nc3ccc4ccc5ccc(-c6cccc(-c7ccc8c(c7)C(c7ccccc7)(c7ccccc7)c7c-8ccc8ccccc78)c6)cc5c4c3s2)cc1 ; CC1(C)c2ccccc2-c2ccc(N(c3ccc(-c4ccccc4)cc3)c3ccc4ccc5ccc6nc(-c7ccccc7)oc6c5c4c3)cc21]

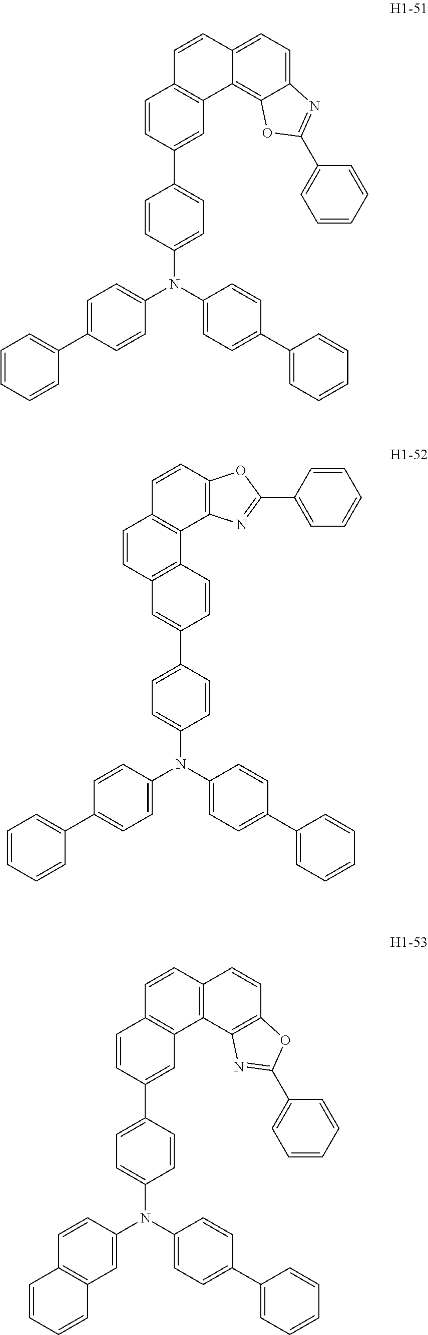 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

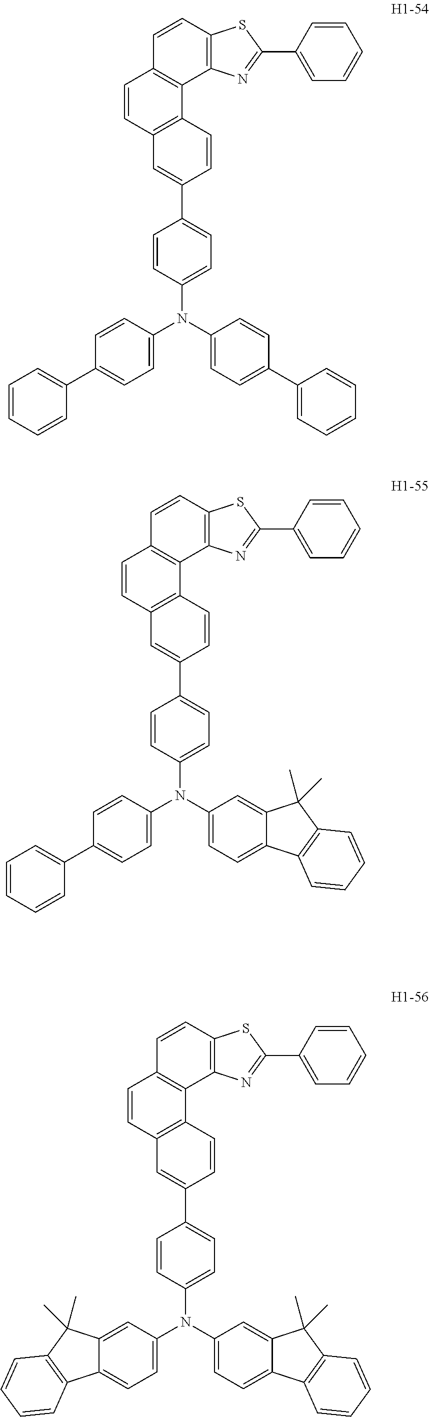 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

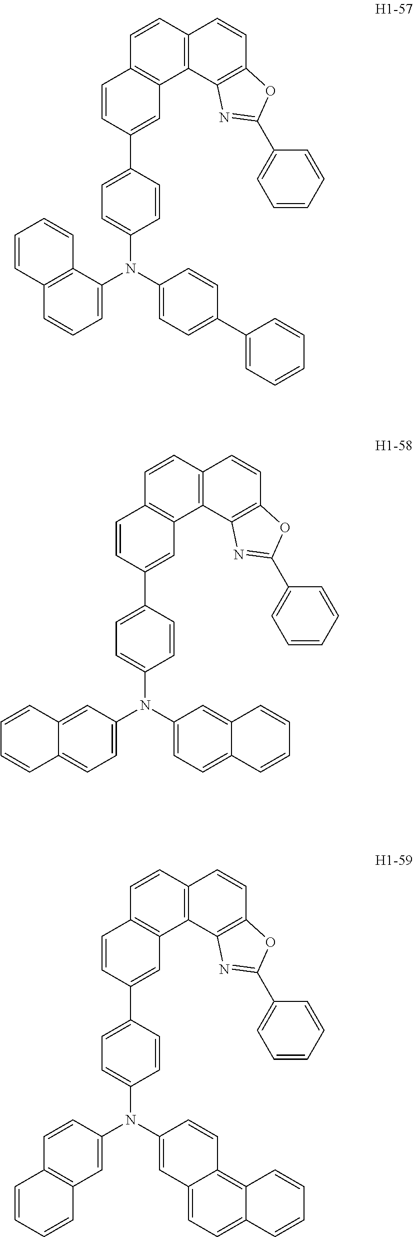 [SMILES: c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1]

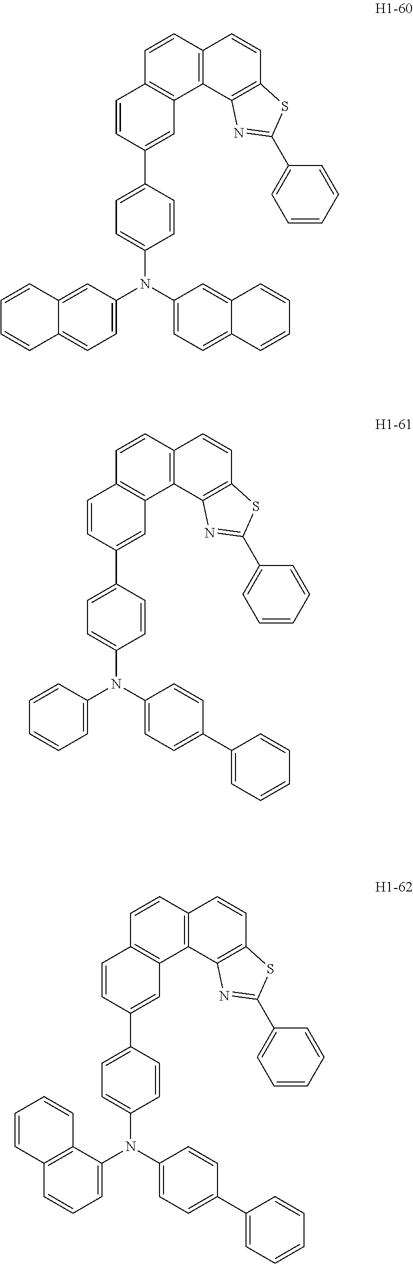 [SMILES: c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1]

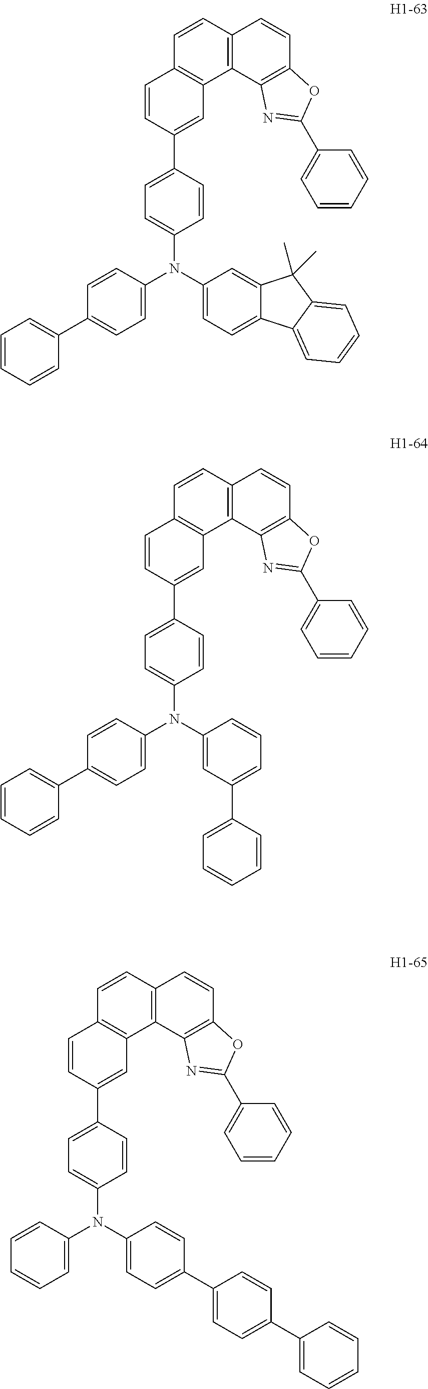 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

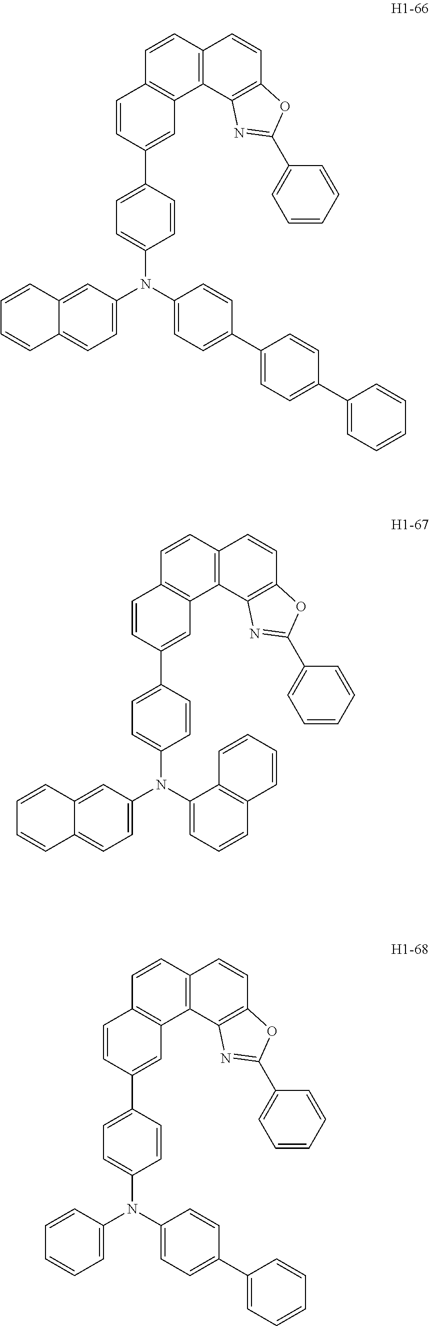 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

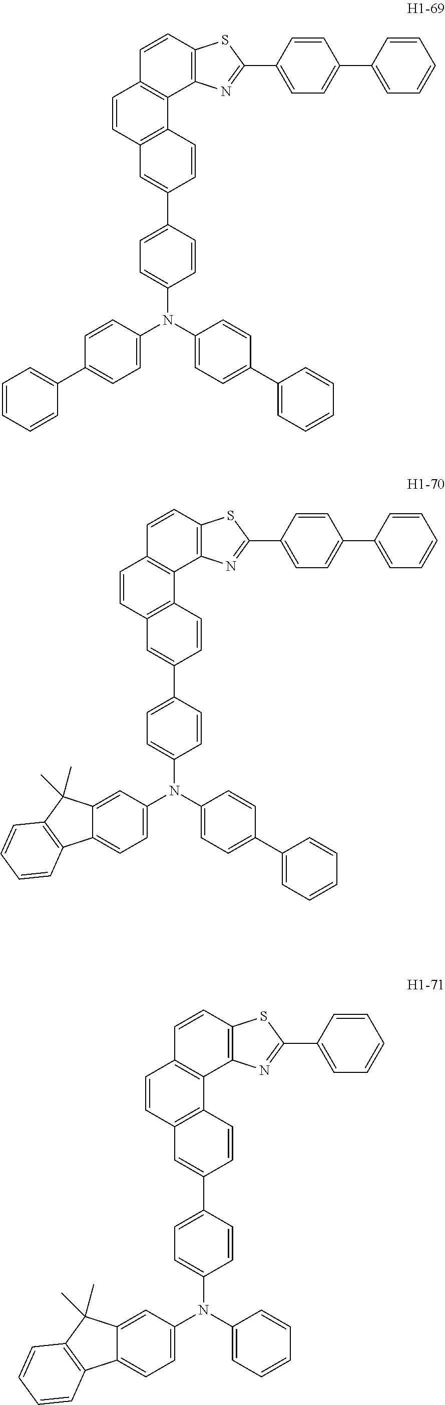 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

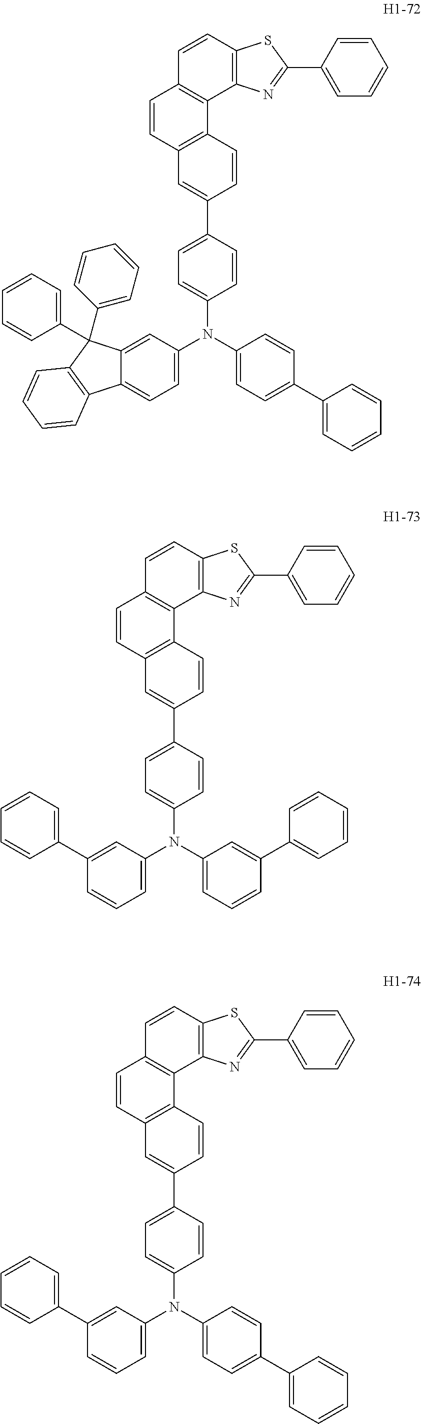 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

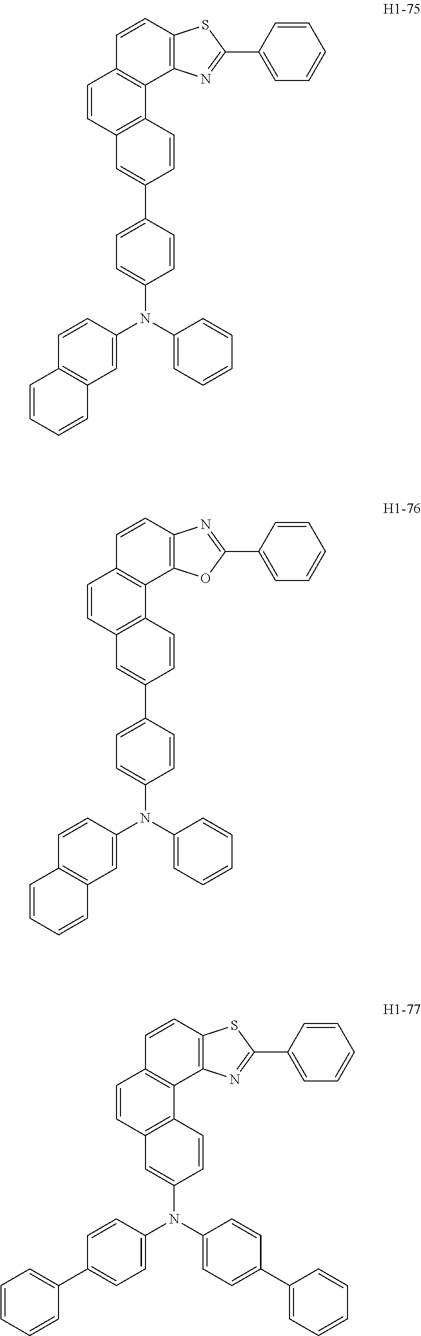 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1]

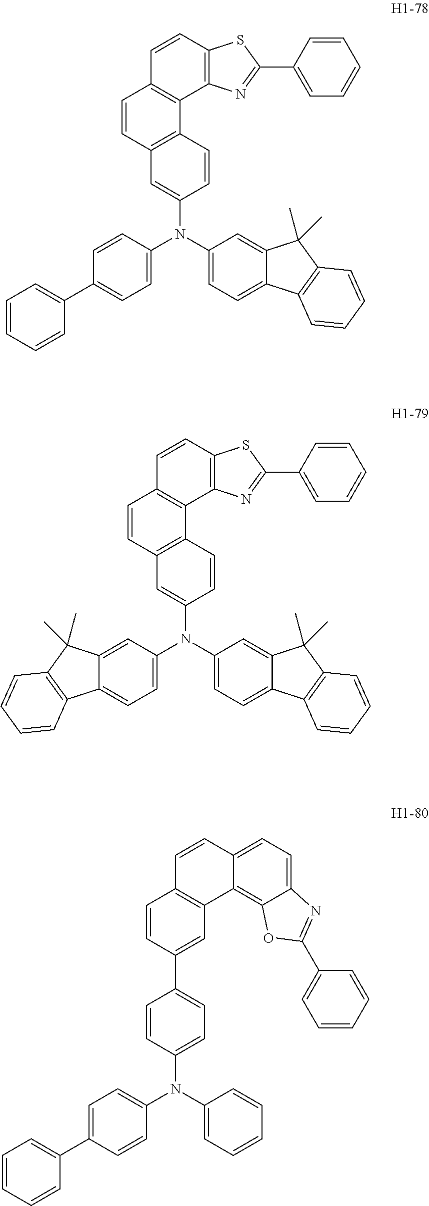 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

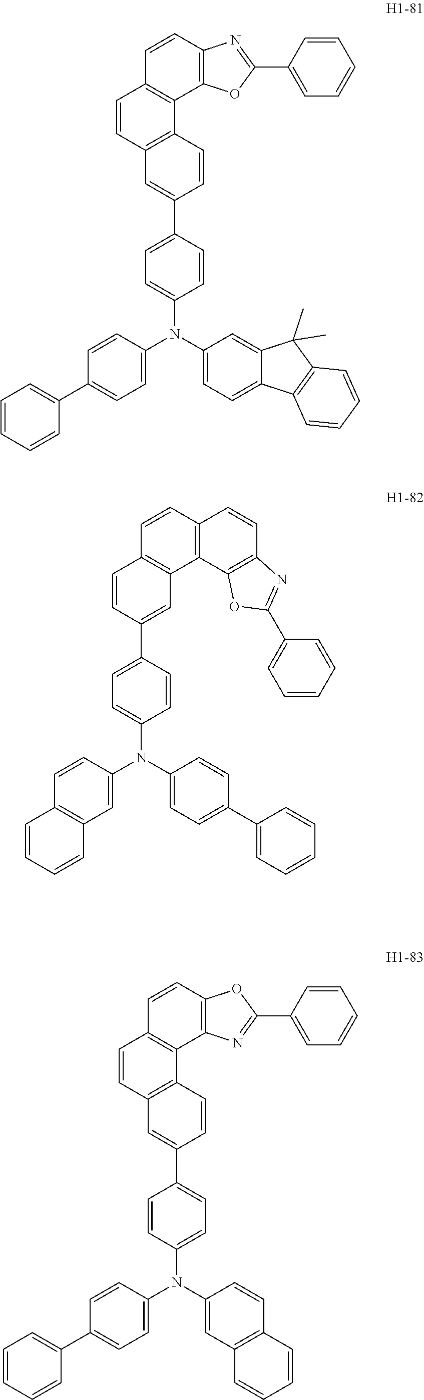 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

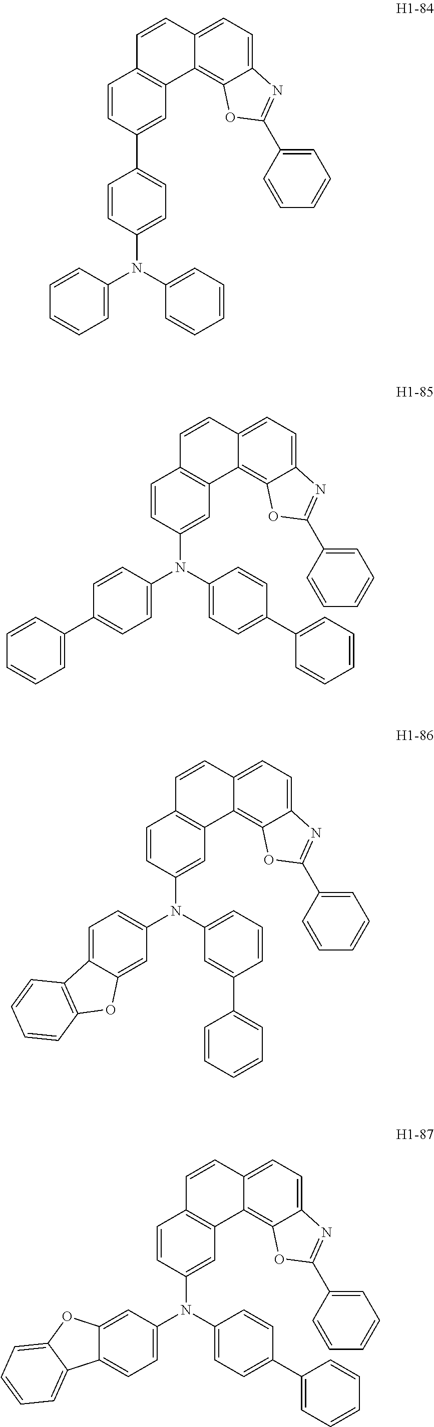 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc4c(c3)oc3ccccc34)c3ccc4ccc5ccc6nc(-c7ccccc7)oc6c5c4c3)cc2)cc1]

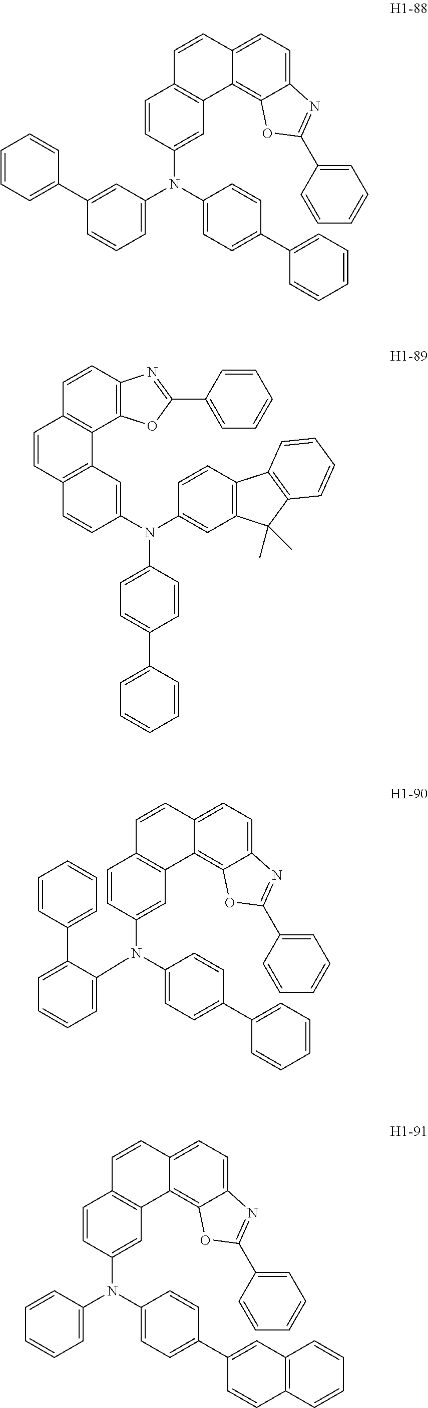 [SMILES: c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1 ; c1ccc(-c2nc3ccc4ccc5ccc(N(c6ccccc6)c6ccc(-c7ccc8ccccc8c7)cc6)cc5c4c3o2)cc1]

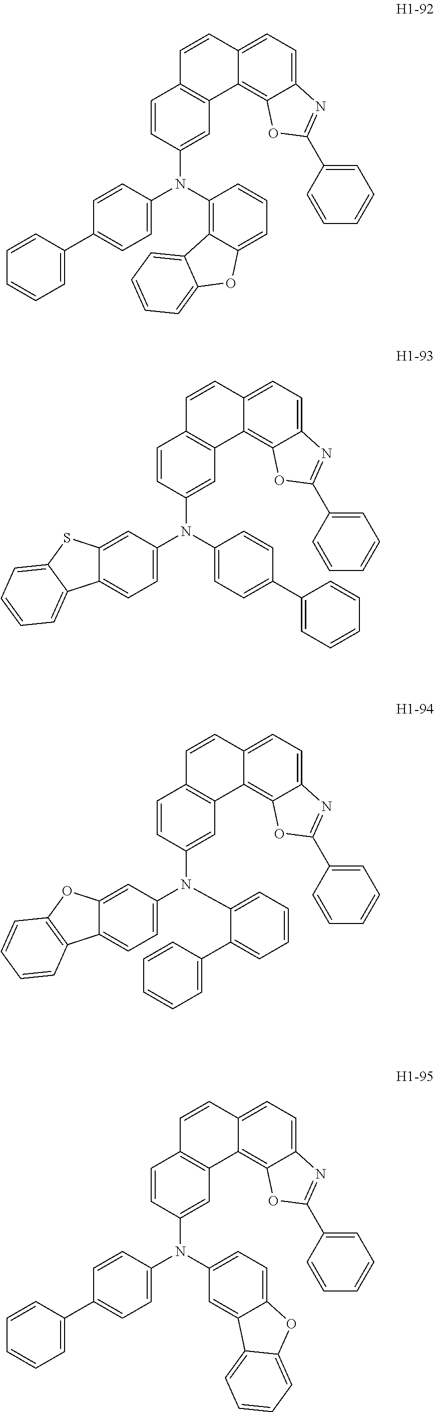 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1]

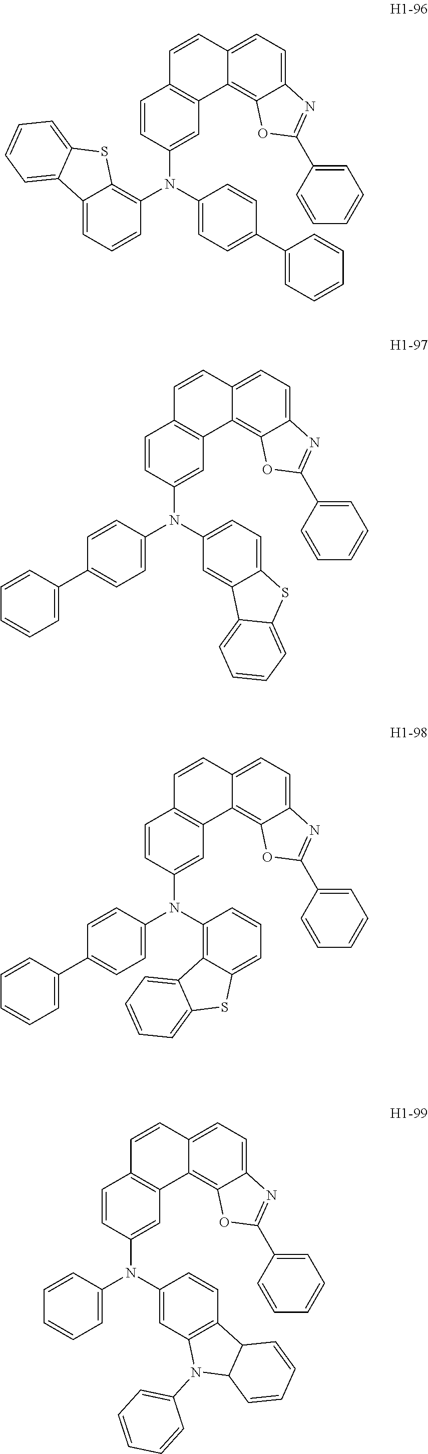 [SMILES: c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1]

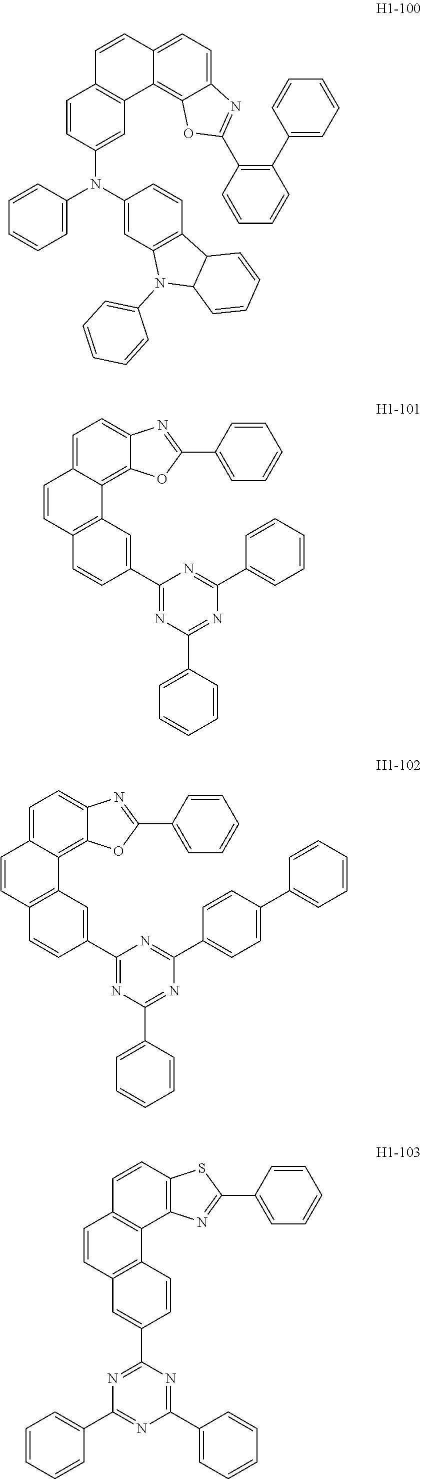 [SMILES: C1=CC2c3ccc(N(c4ccccc4)c4ccc5ccc6ccc7nc(-c8ccccc8-c8ccccc8)oc7c6c5c4)cc3N(c3ccccc3)C2C=C1 ; c1ccc(-c2nc(-c3ccccc3)nc(-c3ccc4ccc5ccc6nc(-c7ccccc7)oc6c5c4c3)n2)cc1 ; c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccccc3)nc(-c3ccc4c(ccc5ccc6sc(-c7ccccc7)nc6c54)c3)n2)cc1]

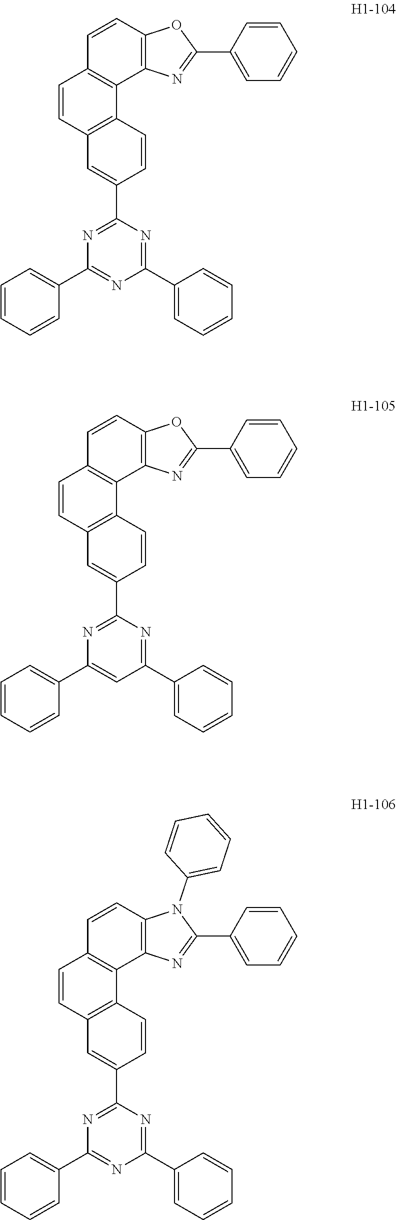 [SMILES: c1ccc(-c2nc(-c3ccccc3)nc(-c3ccc4c(ccc5ccc6oc(-c7ccccc7)nc6c54)c3)n2)cc1 ; c1ccc(-c2cc(-c3ccccc3)nc(-c3ccc4c(ccc5ccc6oc(-c7ccccc7)nc6c54)c3)n2)cc1 ; c1ccc(-c2nc(-c3ccccc3)nc(-c3ccc4c(ccc5ccc6c(nc(-c7ccccc7)n6-c6ccccc6)c54)c3)n2)cc1]

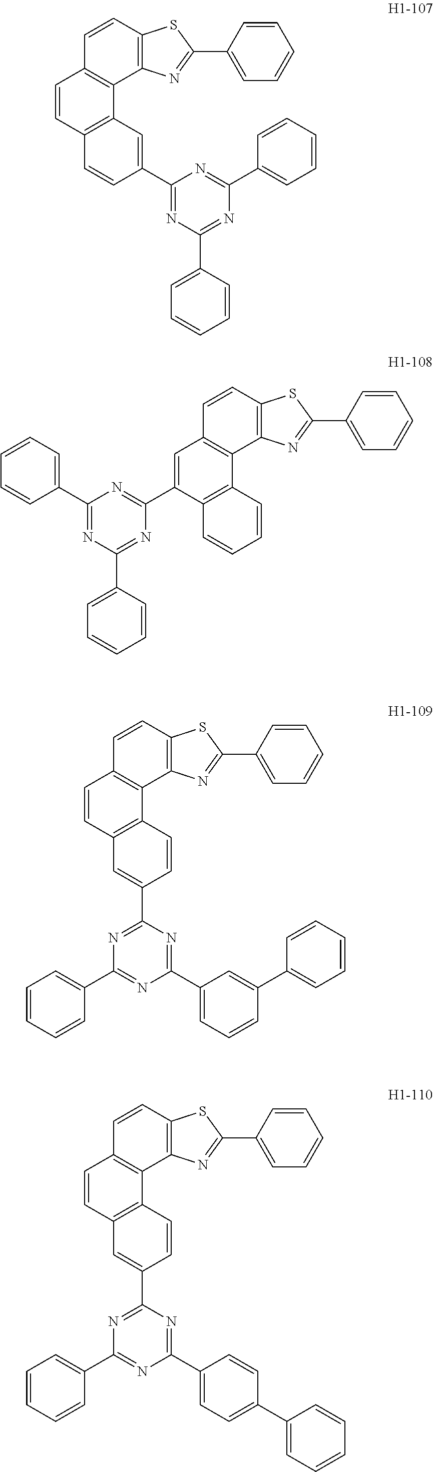 [SMILES: c1ccc(-c2nc(-c3ccccc3)nc(-c3ccc4ccc5ccc6sc(-c7ccccc7)nc6c5c4c3)n2)cc1 ; c1ccc(-c2nc(-c3ccccc3)nc(-c3cc4ccc5sc(-c6ccccc6)nc5c4c4ccccc34)n2)cc1 ; c1ccc(-c2cccc(-c3nc(-c4ccccc4)nc(-c4ccc5c(ccc6ccc7sc(-c8ccccc8)nc7c65)c4)n3)c2)cc1 ; c1ccc(-c2ccc(-c3nc(-c4ccccc4)nc(-c4ccc5c(ccc6ccc7sc(-c8ccccc8)nc7c65)c4)n3)cc2)cc1]

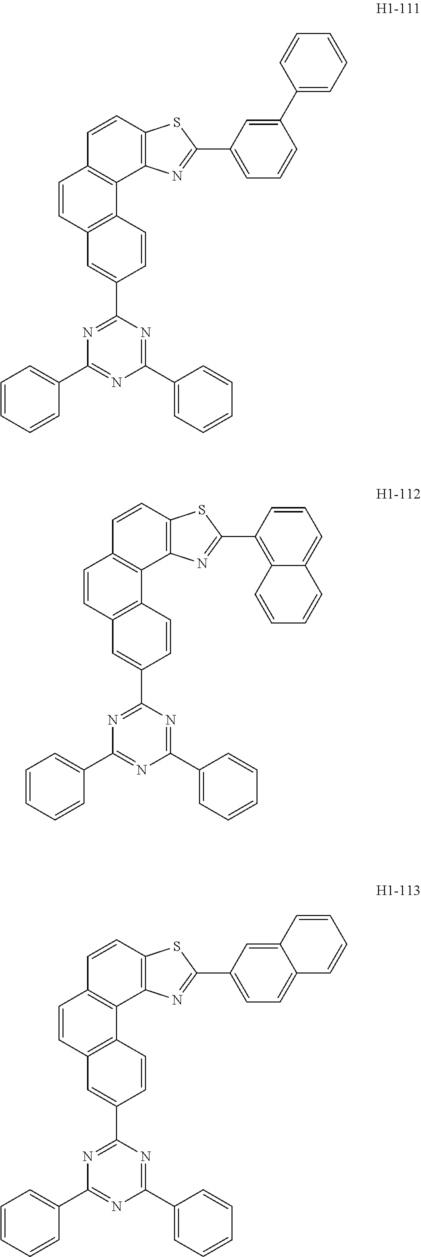 [SMILES: c1ccc(-c2cccc(-c3nc4c(ccc5ccc6cc(-c7nc(-c8ccccc8)nc(-c8ccccc8)n7)ccc6c54)s3)c2)cc1 ; c1ccc(-c2nc(-c3ccccc3)nc(-c3ccc4c(ccc5ccc6sc(-c7cccc8ccccc78)nc6c54)c3)n2)cc1 ; c1ccc(-c2nc(-c3ccccc3)nc(-c3ccc4c(ccc5ccc6sc(-c7ccc8ccccc8c7)nc6c54)c3)n2)cc1]

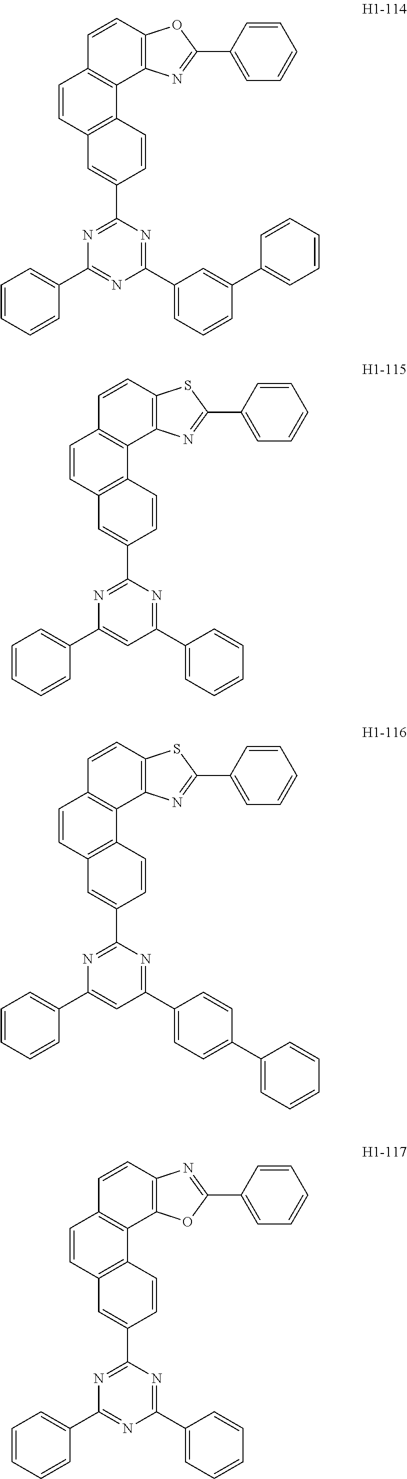 [SMILES: c1ccc(-c2cccc(-c3nc(-c4ccccc4)nc(-c4ccc5c(ccc6ccc7oc(-c8ccccc8)nc7c65)c4)n3)c2)cc1 ; c1ccc(-c2cc(-c3ccccc3)nc(-c3ccc4c(ccc5ccc6sc(-c7ccccc7)nc6c54)c3)n2)cc1 ; c1ccc(-c2ccc(-c3cc(-c4ccccc4)nc(-c4ccc5c(ccc6ccc7sc(-c8ccccc8)nc7c65)c4)n3)cc2)cc1 ; c1ccc(-c2nc(-c3ccccc3)nc(-c3ccc4c(ccc5ccc6nc(-c7ccccc7)oc6c54)c3)n2)cc1]

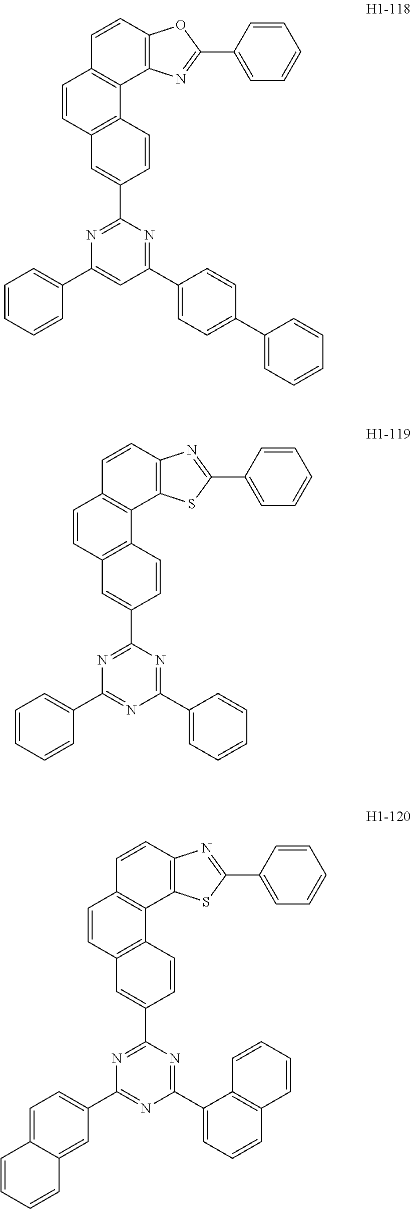 [SMILES: c1ccc(-c2ccc(-c3cc(-c4ccccc4)nc(-c4ccc5c(ccc6ccc7oc(-c8ccccc8)nc7c65)c4)n3)cc2)cc1 ; c1ccc(-c2nc(-c3ccccc3)nc(-c3ccc4c(ccc5ccc6nc(-c7ccccc7)sc6c54)c3)n2)cc1 ; c1ccc(-c2nc3ccc4ccc5cc(-c6nc(-c7ccc8ccccc8c7)nc(-c7cccc8ccccc78)n6)ccc5c4c3s2)cc1]

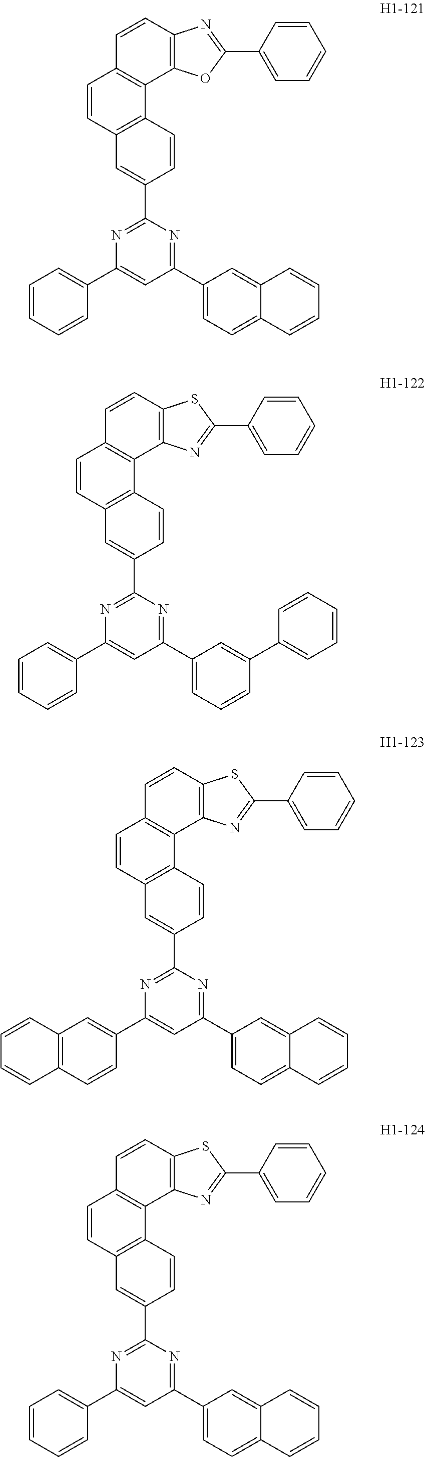 [SMILES: c1ccc(-c2cc(-c3ccc4ccccc4c3)nc(-c3ccc4c(ccc5ccc6nc(-c7ccccc7)oc6c54)c3)n2)cc1 ; c1ccc(-c2cccc(-c3cc(-c4ccccc4)nc(-c4ccc5c(ccc6ccc7sc(-c8ccccc8)nc7c65)c4)n3)c2)cc1 ; c1ccc(-c2nc3c(ccc4ccc5cc(-c6nc(-c7ccc8ccccc8c7)cc(-c7ccc8ccccc8c7)n6)ccc5c43)s2)cc1 ; c1ccc(-c2cc(-c3ccc4ccccc4c3)nc(-c3ccc4c(ccc5ccc6sc(-c7ccccc7)nc6c54)c3)n2)cc1]

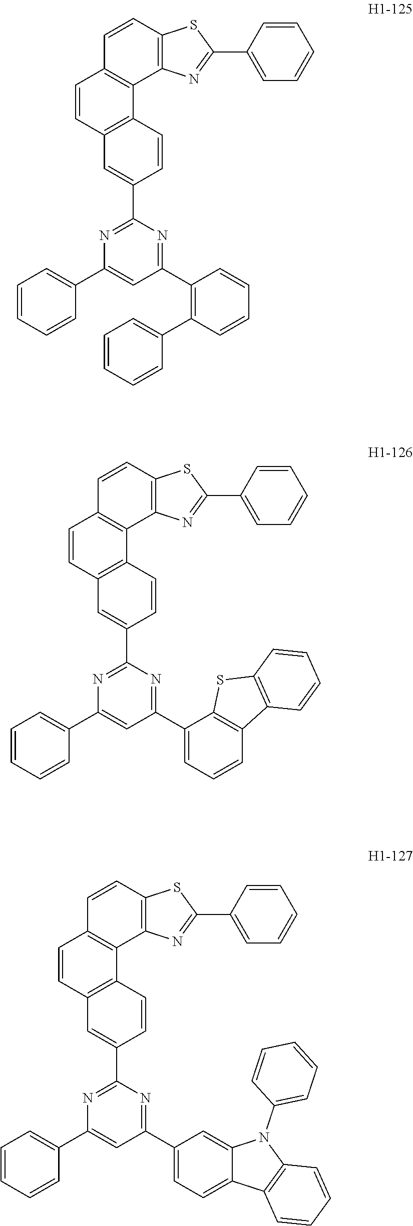 [SMILES: c1ccc(-c2cc(-c3ccccc3-c3ccccc3)nc(-c3ccc4c(ccc5ccc6sc(-c7ccccc7)nc6c54)c3)n2)cc1 ; c1ccc(-c2cc(-c3cccc4c3sc3ccccc34)nc(-c3ccc4c(ccc5ccc6sc(-c7ccccc7)nc6c54)c3)n2)cc1 ; c1ccc(-c2cc(-c3ccc4c5ccccc5n(-c5ccccc5)c4c3)nc(-c3ccc4c(ccc5ccc6sc(-c7ccccc7)nc6c54)c3)n2)cc1]

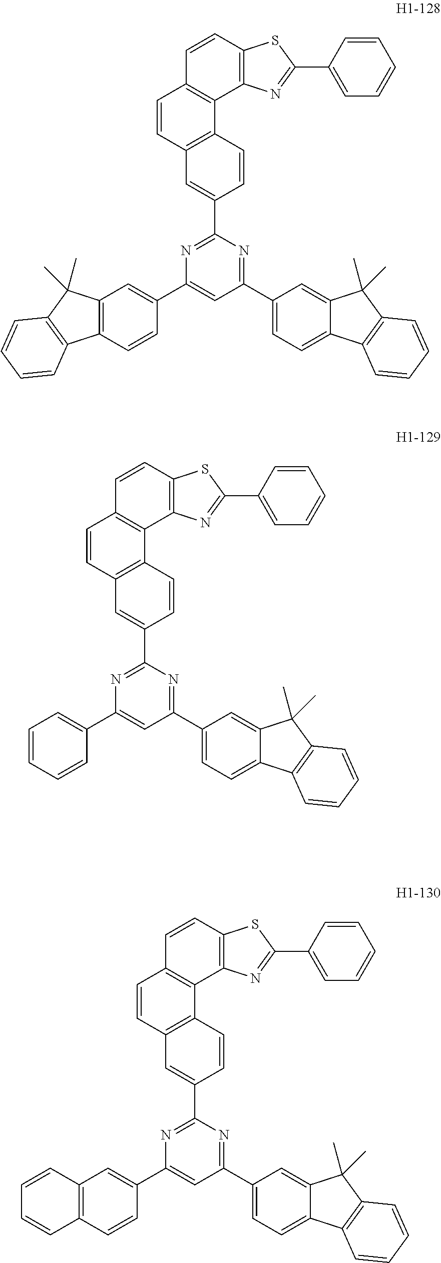 [SMILES: CC1(C)c2ccccc2-c2ccc(-c3cc(-c4ccc5c(c4)C(C)(C)c4ccccc4-5)nc(-c4ccc5c(ccc6ccc7sc(-c8ccccc8)nc7c65)c4)n3)cc21 ; CC1(C)c2ccccc2-c2ccc(-c3cc(-c4ccccc4)nc(-c4ccc5c(ccc6ccc7sc(-c8ccccc8)nc7c65)c4)n3)cc21 ; CC1(C)c2ccccc2-c2ccc(-c3cc(-c4ccc5ccccc5c4)nc(-c4ccc5c(ccc6ccc7sc(-c8ccccc8)nc7c65)c4)n3)cc21]

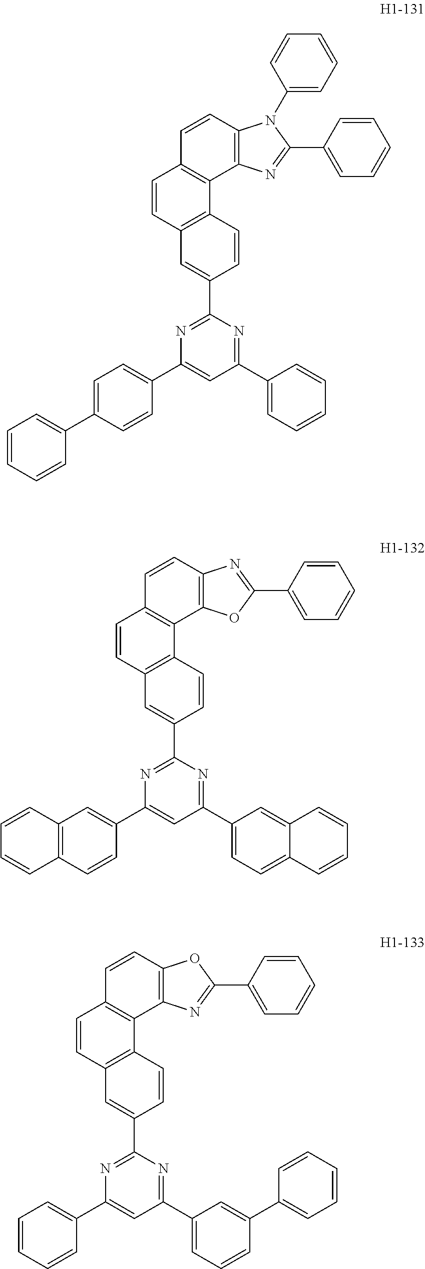 [SMILES: c1ccc(-c2ccc(-c3cc(-c4ccccc4)nc(-c4ccc5c(ccc6ccc7c(nc(-c8ccccc8)n7-c7ccccc7)c65)c4)n3)cc2)cc1 ; c1ccc(-c2nc3ccc4ccc5cc(-c6nc(-c7ccc8ccccc8c7)cc(-c7ccc8ccccc8c7)n6)ccc5c4c3o2)cc1 ; c1ccc(-c2cccc(-c3cc(-c4ccccc4)nc(-c4ccc5c(ccc6ccc7oc(-c8ccccc8)nc7c65)c4)n3)c2)cc1]

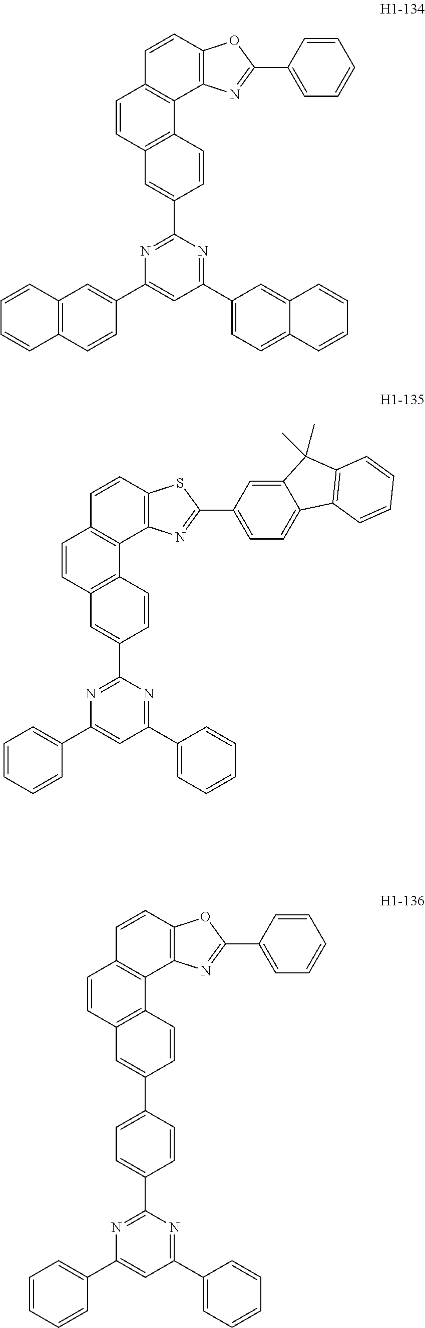 [SMILES: c1ccc(-c2nc3c(ccc4ccc5cc(-c6nc(-c7ccc8ccccc8c7)cc(-c7ccc8ccccc8c7)n6)ccc5c43)o2)cc1 ; CC1(C)c2ccccc2-c2ccc(-c3nc4c(ccc5ccc6cc(-c7nc(-c8ccccc8)cc(-c8ccccc8)n7)ccc6c54)s3)cc21 ; c1ccc(-c2cc(-c3ccccc3)nc(-c3ccc(-c4ccc5c(ccc6ccc7oc(-c8ccccc8)nc7c65)c4)cc3)n2)cc1]

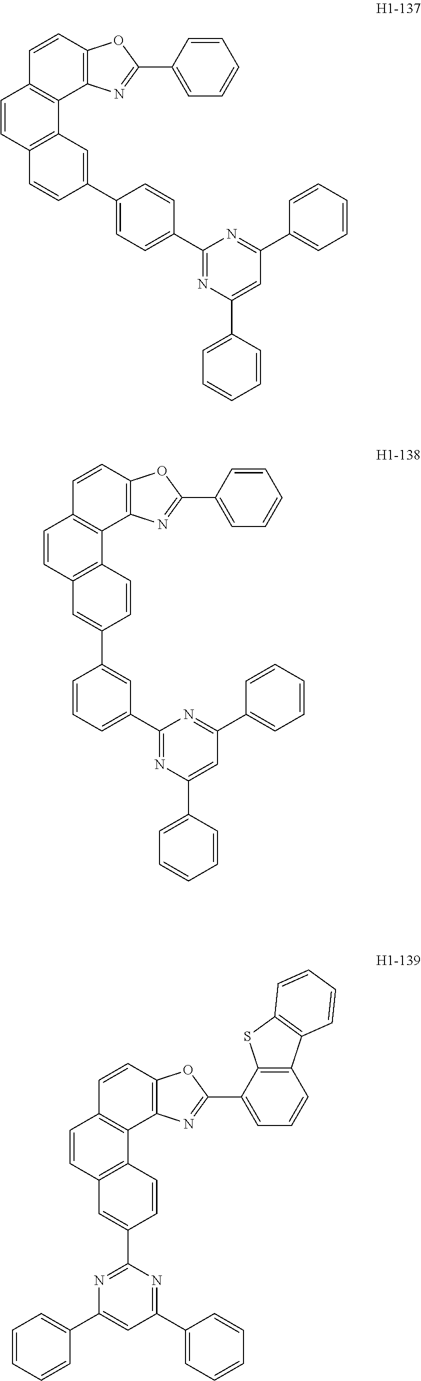 [SMILES: c1ccc(-c2cc(-c3ccccc3)nc(-c3ccc(-c4ccc5ccc6ccc7oc(-c8ccccc8)nc7c6c5c4)cc3)n2)cc1 ; c1ccc(-c2cc(-c3ccccc3)nc(-c3cccc(-c4ccc5c(ccc6ccc7oc(-c8ccccc8)nc7c65)c4)c3)n2)cc1 ; c1ccc(-c2cc(-c3ccccc3)nc(-c3ccc4c(ccc5ccc6oc(-c7cccc8c7sc7ccccc78)nc6c54)c3)n2)cc1]

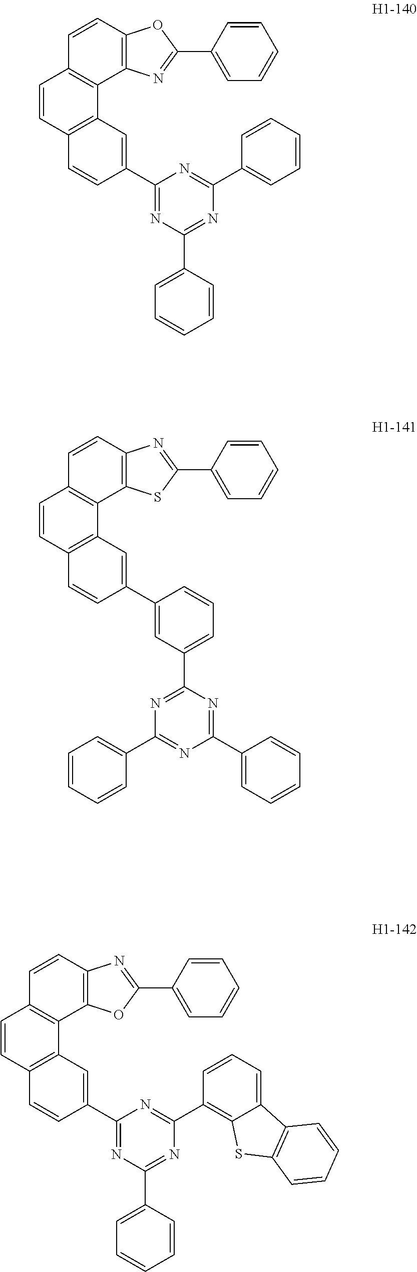 [SMILES: c1ccc(-c2nc(-c3ccccc3)nc(-c3ccc4ccc5ccc6oc(-c7ccccc7)nc6c5c4c3)n2)cc1 ; c1ccc(-c2nc(-c3ccccc3)nc(-c3cccc(-c4ccc5ccc6ccc7nc(-c8ccccc8)sc7c6c5c4)c3)n2)cc1 ; c1ccc(-c2nc(-c3ccc4ccc5ccc6nc(-c7ccccc7)oc6c5c4c3)nc(-c3cccc4c3sc3ccccc34)n2)cc1]

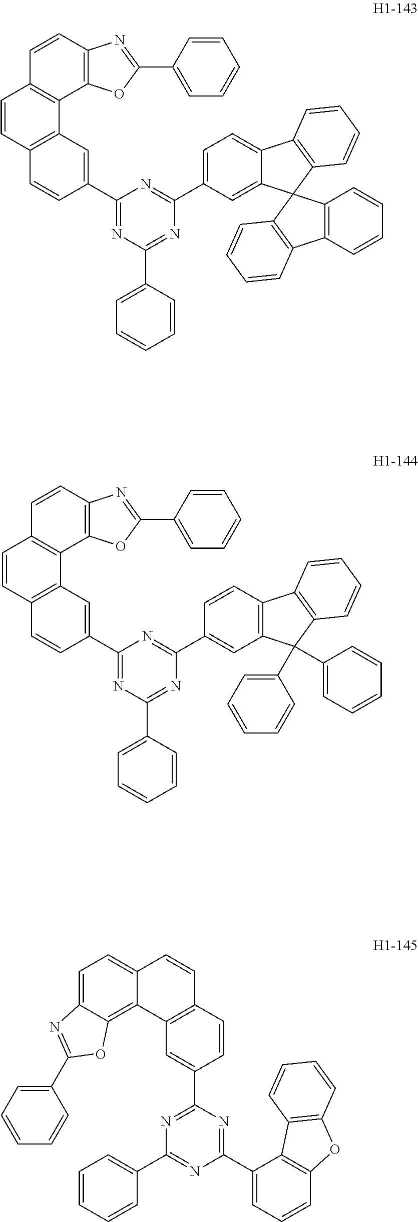 [SMILES: c1ccc(-c2nc(-c3ccc4c(c3)C3(c5ccccc5-c5ccccc53)c3ccccc3-4)nc(-c3ccc4ccc5ccc6nc(-c7ccccc7)oc6c5c4c3)n2)cc1 ; c1ccc(-c2nc(-c3ccc4c(c3)C(c3ccccc3)(c3ccccc3)c3ccccc3-4)nc(-c3ccc4ccc5ccc6nc(-c7ccccc7)oc6c5c4c3)n2)cc1 ; c1ccc(-c2nc(-c3ccc4ccc5ccc6nc(-c7ccccc7)oc6c5c4c3)nc(-c3cccc4oc5ccccc5c34)n2)cc1]

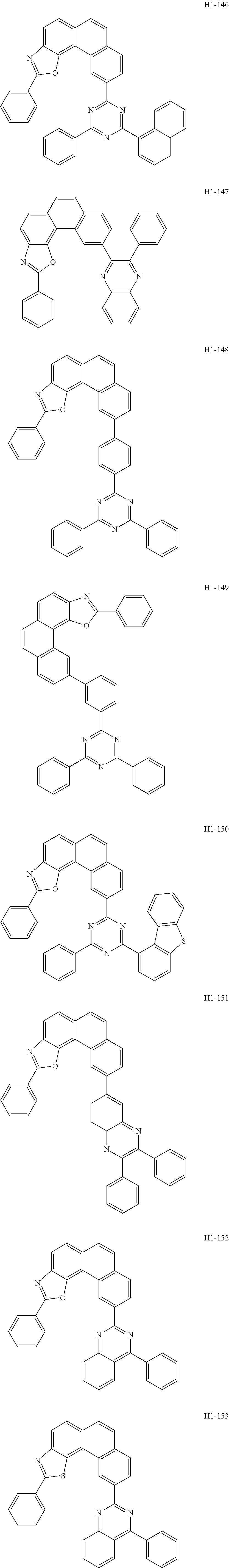

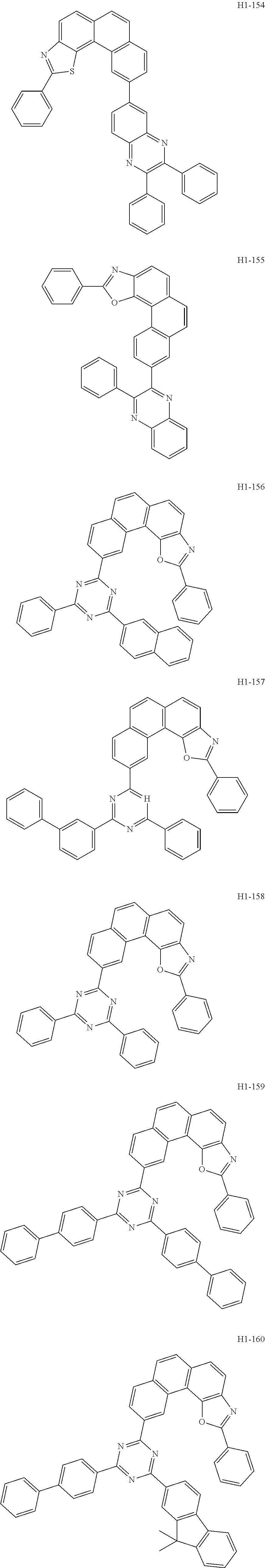

Hereinafter, the compound represented by formula 2 will be described in more detail.

In formula 2, HAr represents a substituted or unsubstituted (3- to 30-membered) heteroaryl containing a nitrogen atom(s). According to one embodiment of the present disclosure, HAr represents a substituted or unsubstituted (5- to 15-membered) heteroaryl containing a nitrogen atom(s). According to another embodiment of the present disclosure, HAr represents a (5- to 15-membered) heteroaryl containing a nitrogen atom(s) and unsubstituted or substituted with a (C6-C20) aryl(s), in which the (C6-C20) aryl may be unsubstituted or substituted with deuterium. Specifically, HAr may be a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted benzothienopyrimidinyl, etc. For example, HAr may be a substituted triazinyl, in which the substituent of the substituted triazinyl may be at least one, preferably at least two, of a phenyl, a naphthyl, a biphenyl, a terphenyl, a phenyl substituted with deuterium, a naphthylphenyl, a naphthyl substituted with deuterium, a phenylnaphthyl, etc.

In formula 2, L₂ represents a single bond, or a substituted or unsubstituted (C6-C30) arylene. According to one embodiment of the present disclosure, L₂ represents a single bond, or a (C6-C18) arylene unsubstituted or substituted with deuterium and/or (C1-C6)alkyl(s). Specifically, L₂ may be a single bond, a phenylene unsubstituted or substituted with deuterium, a naphthylene unsubstituted or substituted with deuterium, a biphenylene unsubstituted or substituted with deuterium, a terphenylene unsubstituted or substituted with deuterium, a phenylene-naphthylene unsubstituted or substituted with deuterium, etc.

In formula 2, R₁ to R₈, each independently, represent hydrogen, deuterium, or a (C6-C30) aryl unsubstituted or substituted with deuterium. According to one embodiment of the present disclosure, R₁ to R₈, each independently, represent hydrogen, deuterium, or a (C6-C18) aryl unsubstituted or substituted with deuterium. Specifically, R₁ to R₈, each independently, may be hydrogen, deuterium, a phenyl, a naphthyl, a biphenyl, a phenyl substituted with deuterium, etc.

The compound represented by formula 2 may be at least one selected from the following compounds, but is not limited thereto.

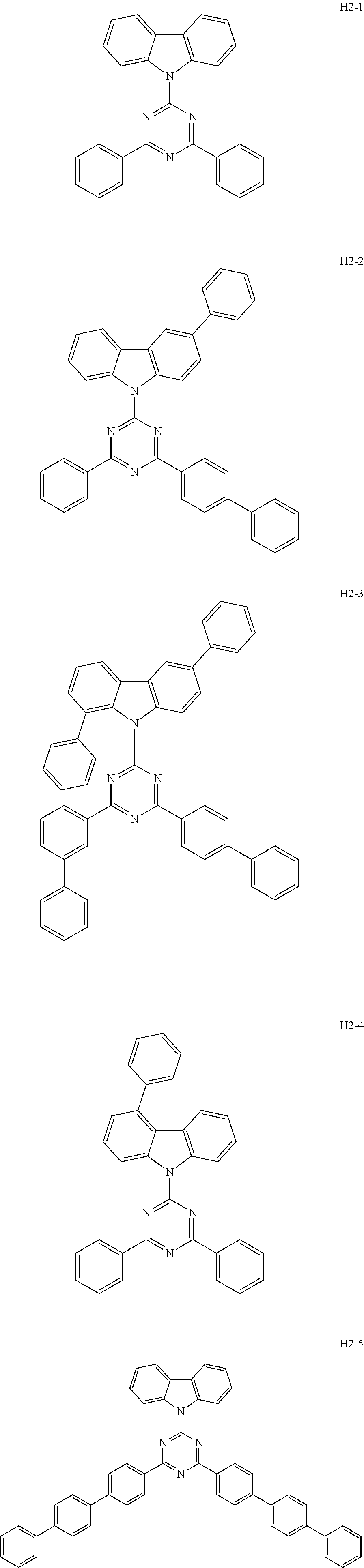 [SMILES: c1ccc(-c2nc(-c3ccccc3)nc(-n3c4ccccc4c4ccccc43)n2)cc1 ; c1ccc(-c2ccc(-c3nc(-c4ccccc4)nc(-n4c5ccccc5c5cc(-c6ccccc6)ccc54)n3)cc2)cc1 ; c1ccc(-c2ccc(-c3nc(-c4cccc(-c5ccccc5)c4)nc(-n4c5ccc(-c6ccccc6)cc5c5cccc(-c6ccccc6)c54)n3)cc2)cc1 ; c1ccc(-c2nc(-c3ccccc3)nc(-n3c4ccccc4c4c(-c5ccccc5)cccc43)n2)cc1 ; c1ccc(-c2ccc(-c3ccc(-c4nc(-c5ccc(-c6ccc(-c7ccccc7)cc6)cc5)nc(-n5c6ccccc6c6ccccc65)n4)cc3)cc2)cc1]

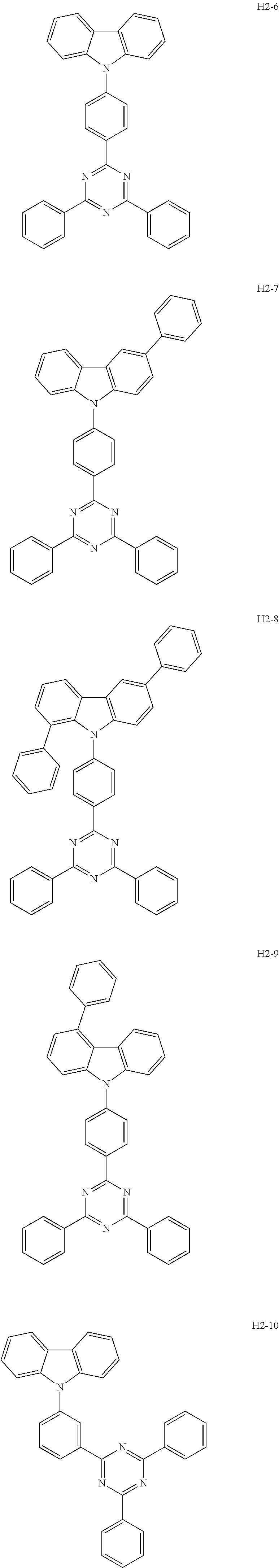 [SMILES: c1ccc(-c2nc(-c3ccccc3)nc(-c3ccc(-n4c5ccccc5c5ccccc54)cc3)n2)cc1 ; c1ccc(-c2ccc3c(c2)c2ccccc2n3-c2ccc(-c3nc(-c4ccccc4)nc(-c4ccccc4)n3)cc2)cc1 ; c1ccc(-c2ccc3c(c2)c2cccc(-c4ccccc4)c2n3-c2ccc(-c3nc(-c4ccccc4)nc(-c4ccccc4)n3)cc2)cc1 ; c1ccc(-c2nc(-c3ccccc3)nc(-c3ccc(-n4c5ccccc5c5c(-c6ccccc6)cccc54)cc3)n2)cc1 ; c1ccc(-c2nc(-c3ccccc3)nc(-c3cccc(-n4c5ccccc5c5ccccc54)c3)n2)cc1]

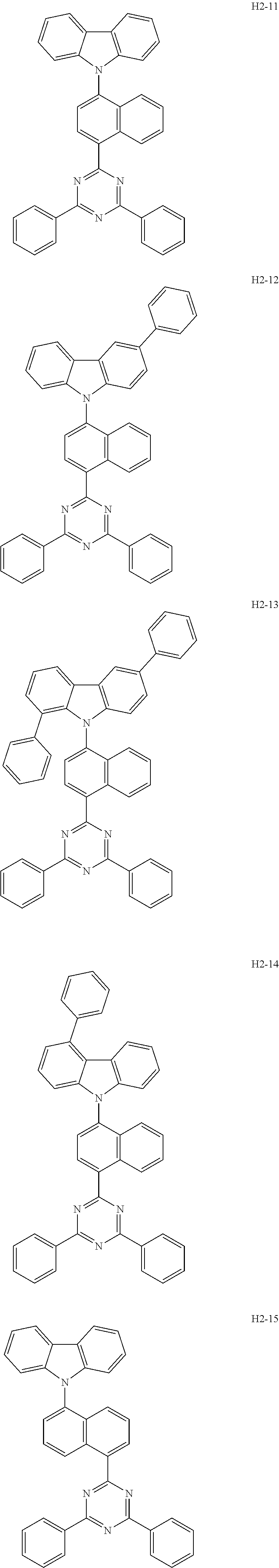

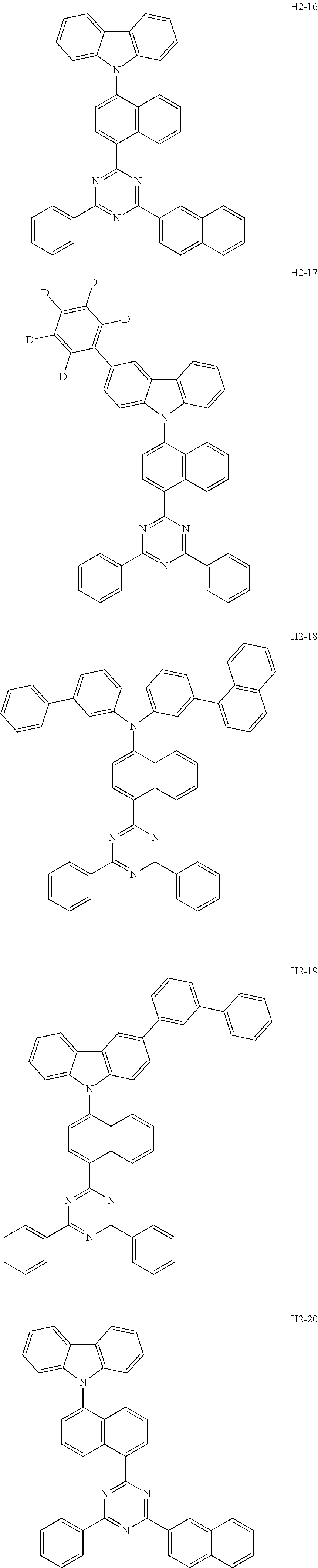 [SMILES: c1ccc(-c2nc(-c3ccc4ccccc4c3)nc(-c3ccc(-n4c5ccccc5c5ccccc54)c4ccccc34)n2)cc1 ; [2H]c1c([2H])c([2H])c(-c2ccc3c(c2)c2ccccc2n3-c2ccc(-c3nc(-c4ccccc4)nc(-c4ccccc4)n3)c3ccccc23)c([2H])c1[2H] ; c1ccc(-c2ccc3c4ccc(-c5cccc6ccccc56)cc4n(-c4ccc(-c5nc(-c6ccccc6)nc(-c6ccccc6)n5)c5ccccc45)c3c2)cc1 ; c1ccc(-c2cccc(-c3ccc4c(c3)c3ccccc3n4-c3ccc(-c4nc(-c5ccccc5)nc(-c5ccccc5)n4)c4ccccc34)c2)cc1 ; c1ccc(-c2nc(-c3ccc4ccccc4c3)nc(-c3cccc4c(-n5c6ccccc6c6ccccc65)cccc34)n2)cc1]

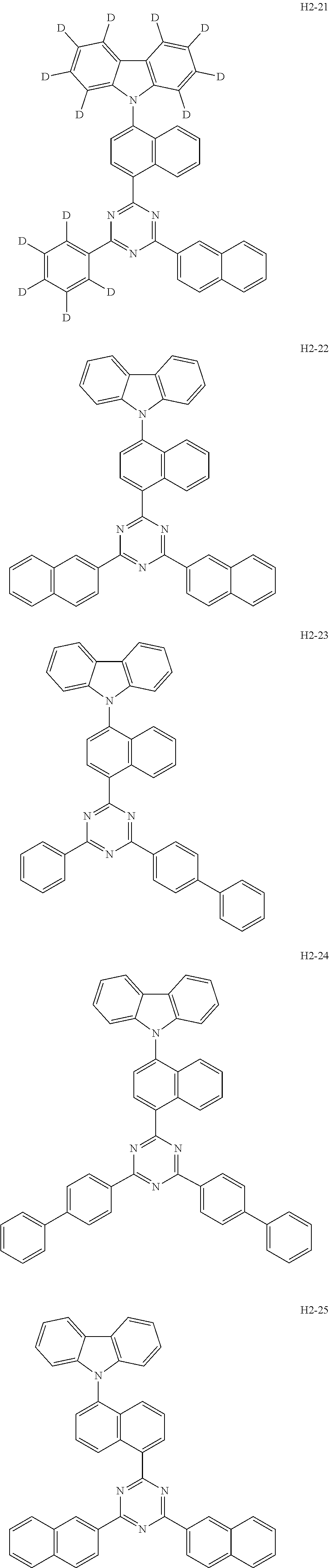 [SMILES: [2H]c1c([2H])c([2H])c(-c2nc(-c3ccc4ccccc4c3)nc(-c3ccc(-n4c5c([2H])c([2H])c([2H])c([2H])c5c5c([2H])c([2H])c([2H])c([2H])c54)c4ccccc34)n2)c([2H])c1[2H] ; c1ccc2cc(-c3nc(-c4ccc5ccccc5c4)nc(-c4ccc(-n5c6ccccc6c6ccccc65)c5ccccc45)n3)ccc2c1 ; c1ccc(-c2ccc(-c3nc(-c4ccccc4)nc(-c4ccc(-n5c6ccccc6c6ccccc65)c5ccccc45)n3)cc2)cc1 ; c1ccc(-c2ccc(-c3nc(-c4ccc(-c5ccccc5)cc4)nc(-c4ccc(-n5c6ccccc6c6ccccc65)c5ccccc45)n3)cc2)cc1 ; c1ccc2cc(-c3nc(-c4ccc5ccccc5c4)nc(-c4cccc5c(-n6c7ccccc7c7ccccc76)cccc45)n3)ccc2c1]

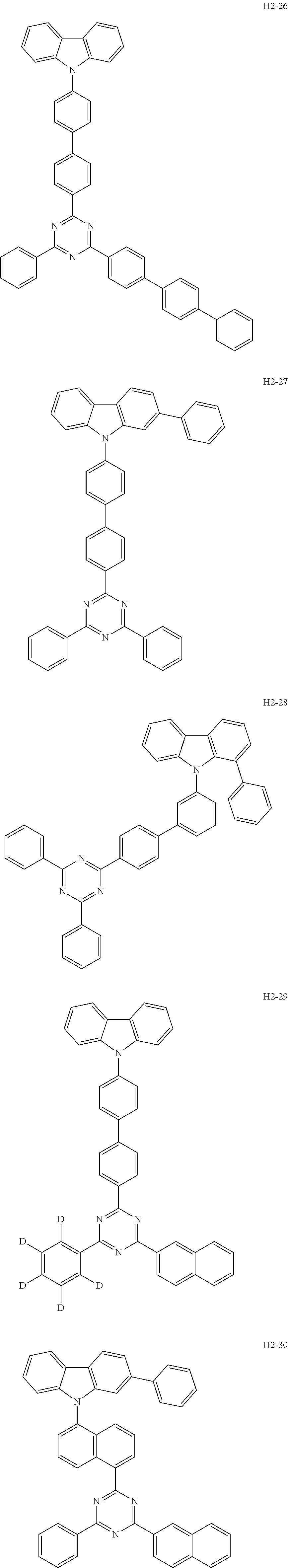 [SMILES: c1ccc(-c2ccc(-c3ccc(-c4nc(-c5ccccc5)nc(-c5ccc(-c6ccc(-n7c8ccccc8c8ccccc87)cc6)cc5)n4)cc3)cc2)cc1 ; c1ccc(-c2ccc3c4ccccc4n(-c4ccc(-c5ccc(-c6nc(-c7ccccc7)nc(-c7ccccc7)n6)cc5)cc4)c3c2)cc1 ; c1ccc(-c2nc(-c3ccccc3)nc(-c3ccc(-c4cccc(-n5c6ccccc6c6cccc(-c7ccccc7)c65)c4)cc3)n2)cc1 ; [2H]c1c([2H])c([2H])c(-c2nc(-c3ccc(-c4ccc(-n5c6ccccc6c6ccccc65)cc4)cc3)nc(-c3ccc4ccccc4c3)n2)c([2H])c1[2H] ; c1ccc(-c2ccc3c4ccccc4n(-c4cccc5c(-c6nc(-c7ccccc7)nc(-c7ccc8ccccc8c7)n6)cccc45)c3c2)cc1]

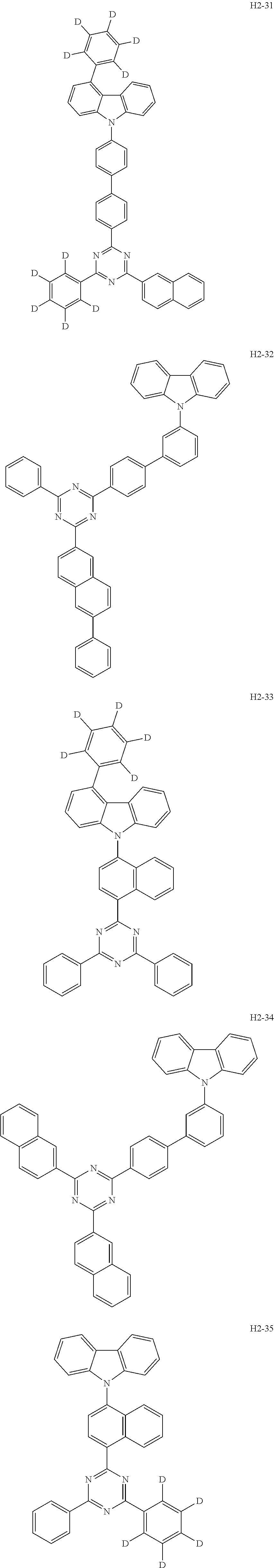

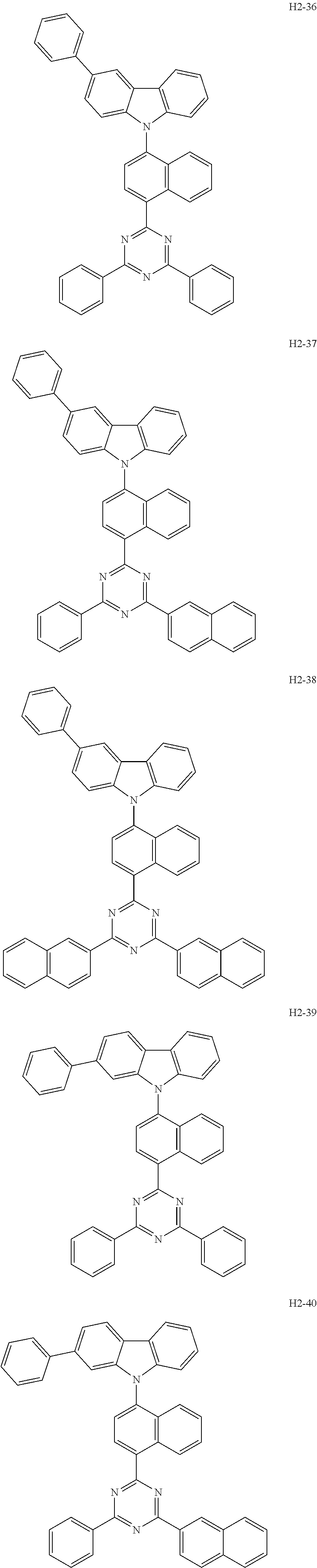 [SMILES: c1ccc(-c2ccc3c(c2)c2ccccc2n3-c2ccc(-c3nc(-c4ccccc4)nc(-c4ccccc4)n3)c3ccccc23)cc1 ; c1ccc(-c2ccc3c(c2)c2ccccc2n3-c2ccc(-c3nc(-c4ccccc4)nc(-c4ccc5ccccc5c4)n3)c3ccccc23)cc1 ; c1ccc(-c2ccc3c(c2)c2ccccc2n3-c2ccc(-c3nc(-c4ccc5ccccc5c4)nc(-c4ccc5ccccc5c4)n3)c3ccccc23)cc1 ; c1ccc(-c2ccc3c4ccccc4n(-c4ccc(-c5nc(-c6ccccc6)nc(-c6ccccc6)n5)c5ccccc45)c3c2)cc1 ; c1ccc(-c2ccc3c4ccccc4n(-c4ccc(-c5nc(-c6ccccc6)nc(-c6ccc7ccccc7c6)n5)c5ccccc45)c3c2)cc1]

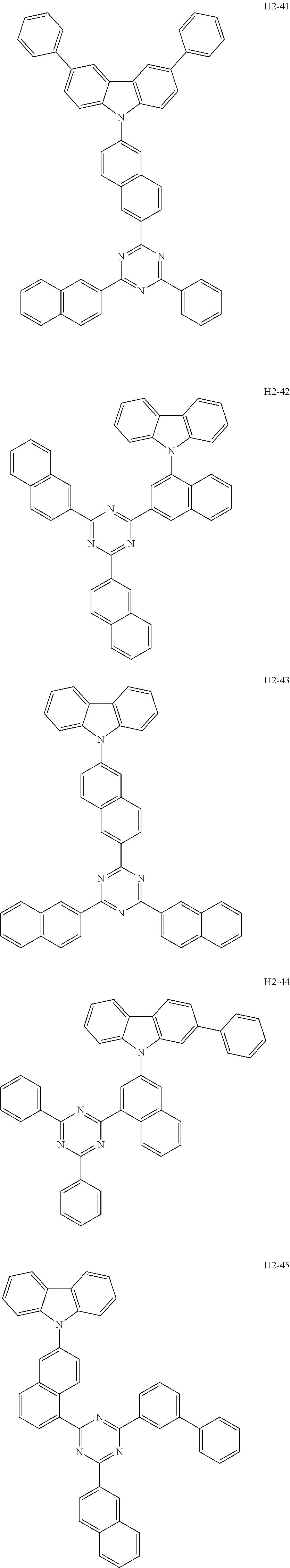

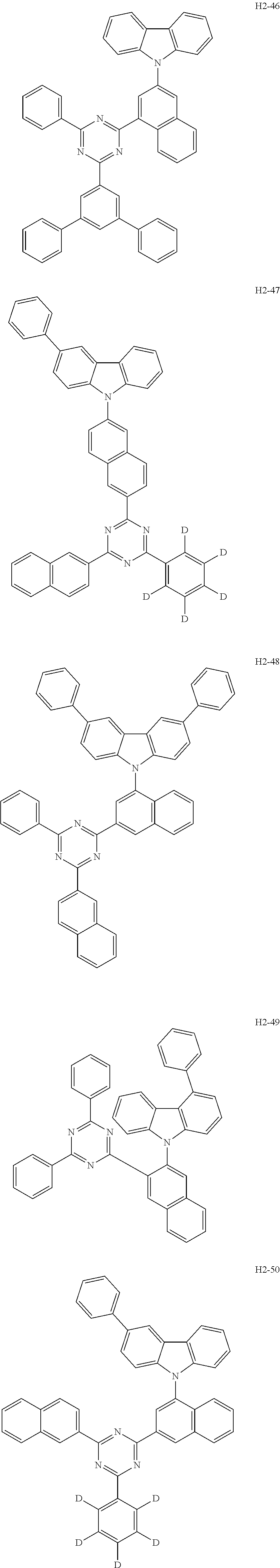

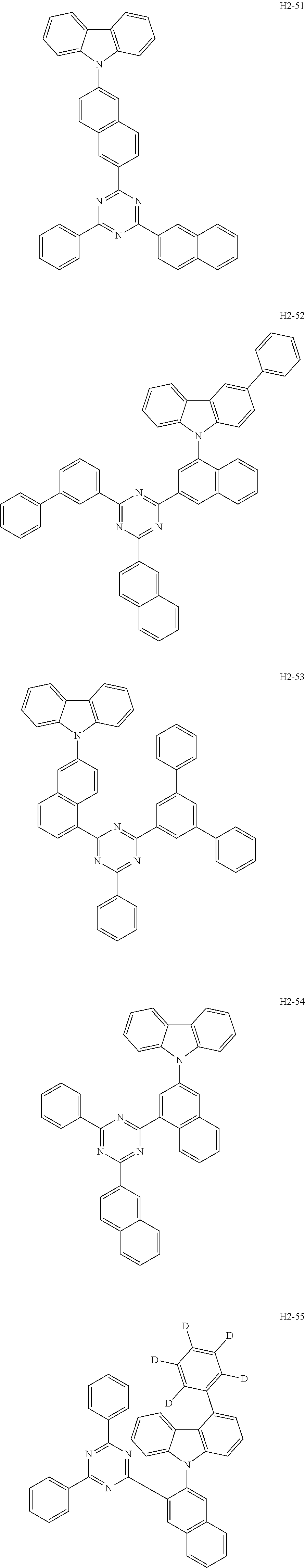

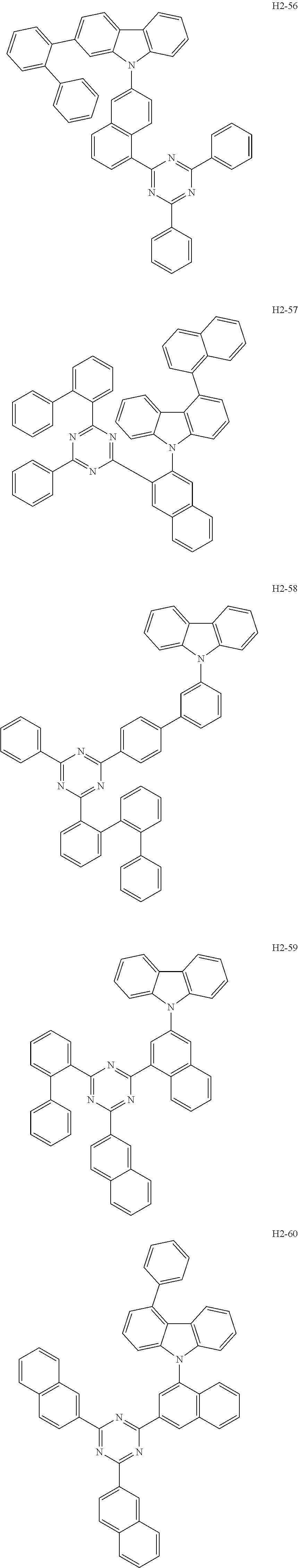

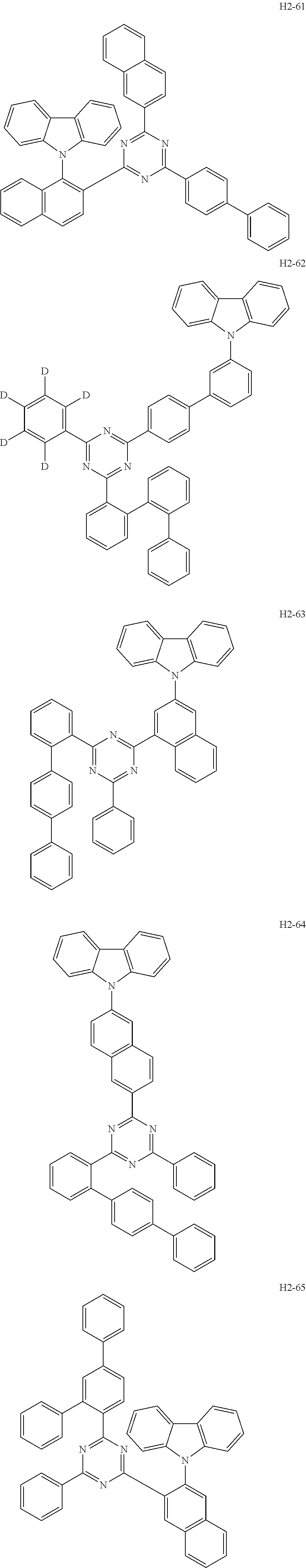

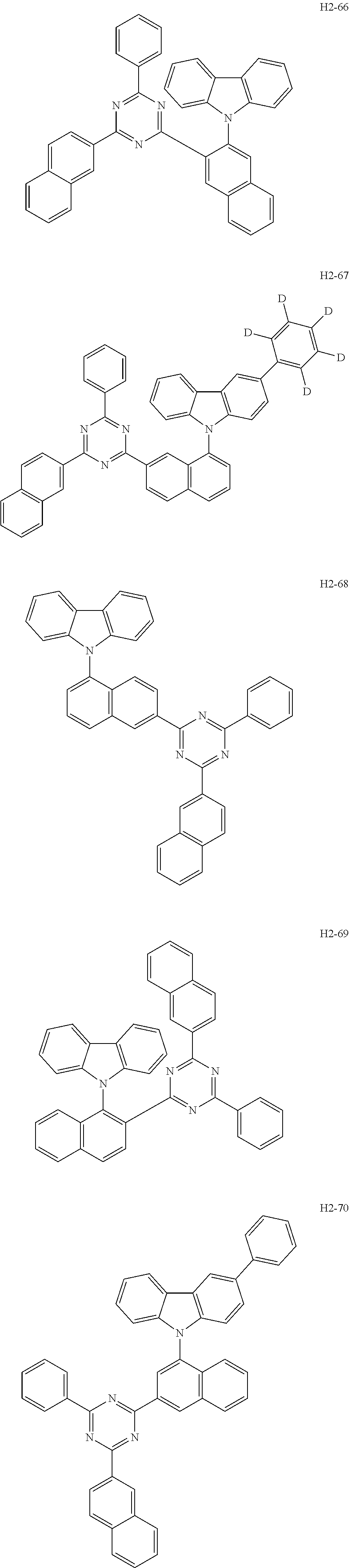

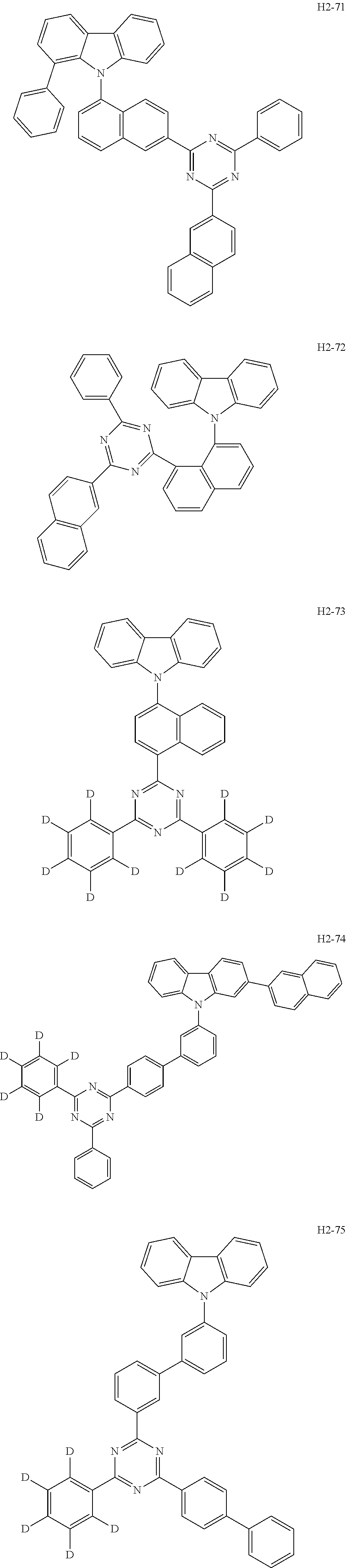

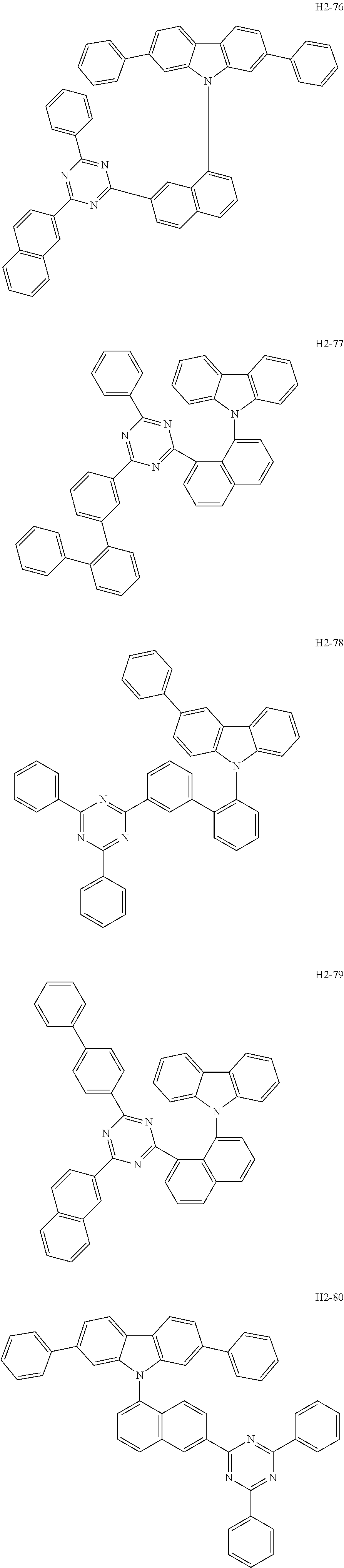 [SMILES: c1ccc(-c2ccc3c4ccc(-c5ccccc5)cc4n(-c4cccc5ccc(-c6nc(-c7ccccc7)nc(-c7ccc8ccccc8c7)n6)cc45)c3c2)cc1 ; c1ccc(-c2nc(-c3cccc(-c4ccccc4-c4ccccc4)c3)nc(-c3cccc4cccc(-n5c6ccccc6c6ccccc65)c34)n2)cc1 ; c1ccc(-c2ccc3c(c2)c2ccccc2n3-c2ccccc2-c2cccc(-c3nc(-c4ccccc4)nc(-c4ccccc4)n3)c2)cc1 ; c1ccc(-c2ccc(-c3nc(-c4ccc5ccccc5c4)nc(-c4cccc5cccc(-n6c7ccccc7c7ccccc76)c45)n3)cc2)cc1 ; c1ccc(-c2ccc3c4ccc(-c5ccccc5)cc4n(-c4cccc5cc(-c6nc(-c7ccccc7)nc(-c7ccccc7)n6)ccc45)c3c2)cc1]

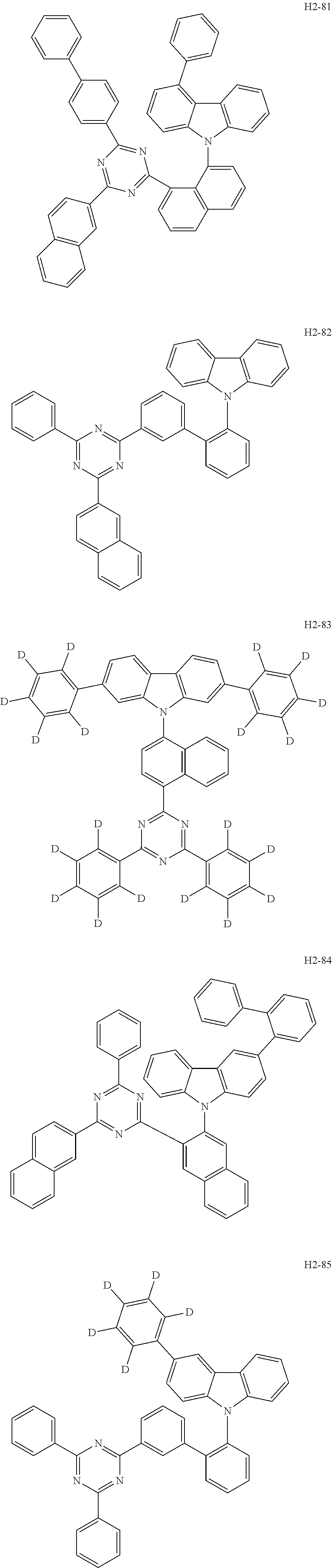

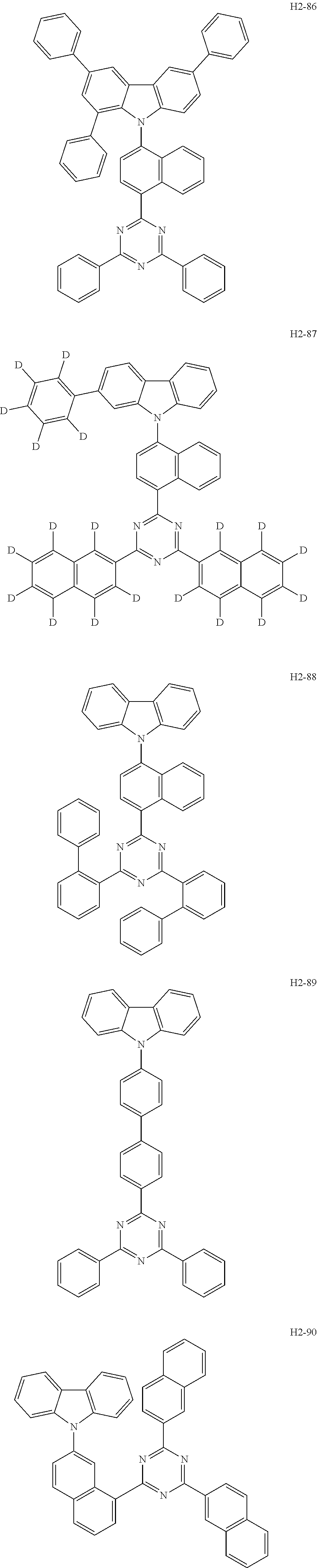

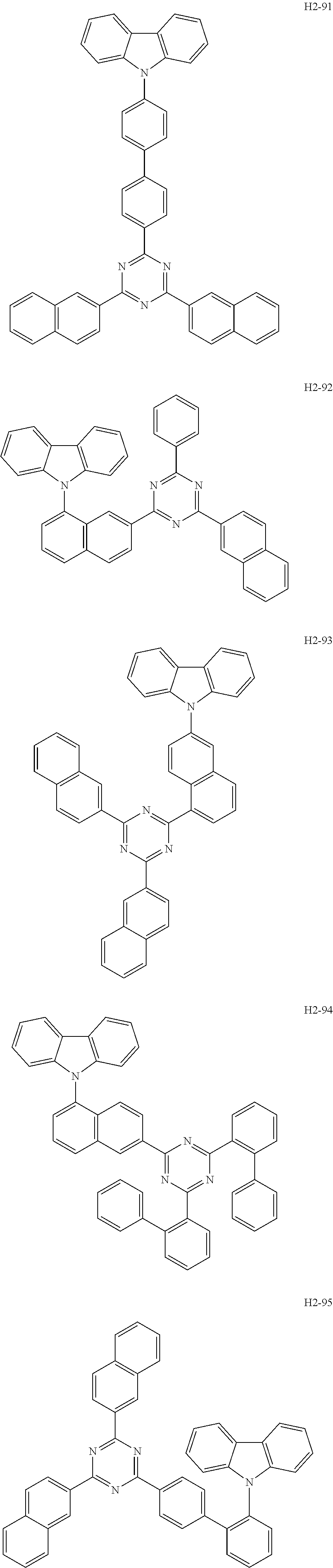 [SMILES: c1ccc2cc(-c3nc(-c4ccc(-c5ccc(-n6c7ccccc7c7ccccc76)cc5)cc4)nc(-c4ccc5ccccc5c4)n3)ccc2c1 ; c1ccc(-c2nc(-c3ccc4ccccc4c3)nc(-c3ccc4cccc(-n5c6ccccc6c6ccccc65)c4c3)n2)cc1 ; c1ccc2cc(-c3nc(-c4ccc5ccccc5c4)nc(-c4cccc5cc(-n6c7ccccc7c7ccccc76)ccc45)n3)ccc2c1 ; c1ccc(-c2ccccc2-c2nc(-c3ccc4c(-n5c6ccccc6c6ccccc65)cccc4c3)nc(-c3ccccc3-c3ccccc3)n2)cc1 ; c1ccc(-n2c3ccccc3c3ccccc32)c(-c2ccc(-c3nc(-c4ccc5ccccc5c4)nc(-c4ccc5ccccc5c4)n3)cc2)c1]

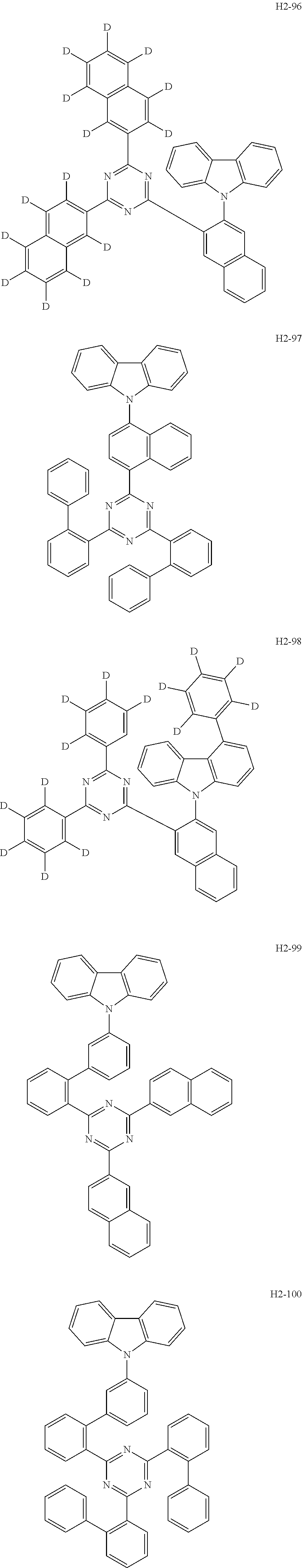

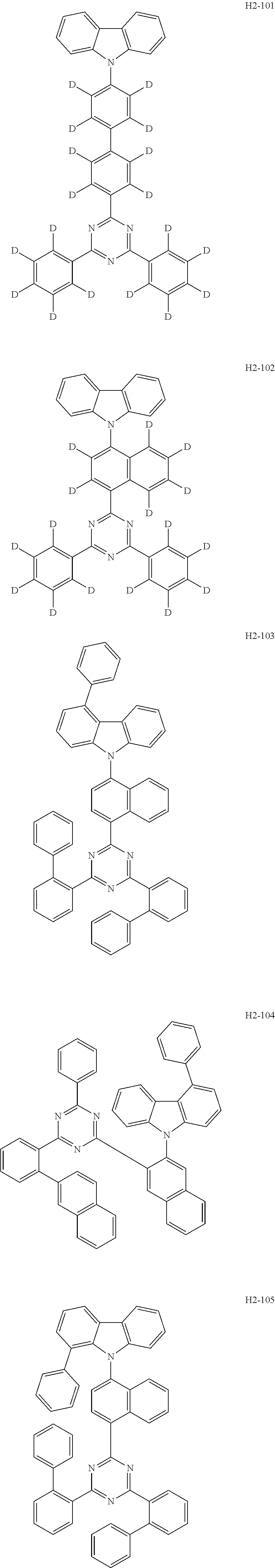

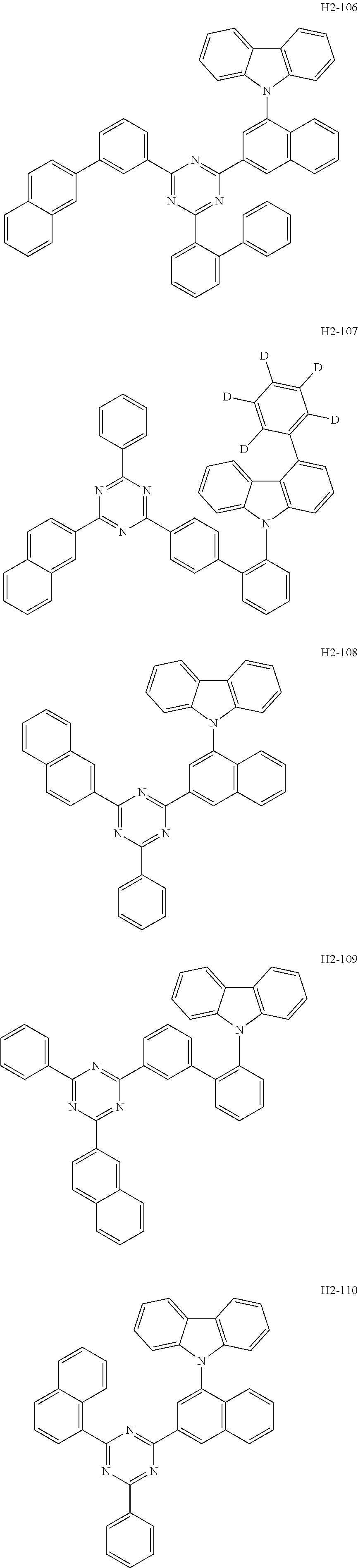

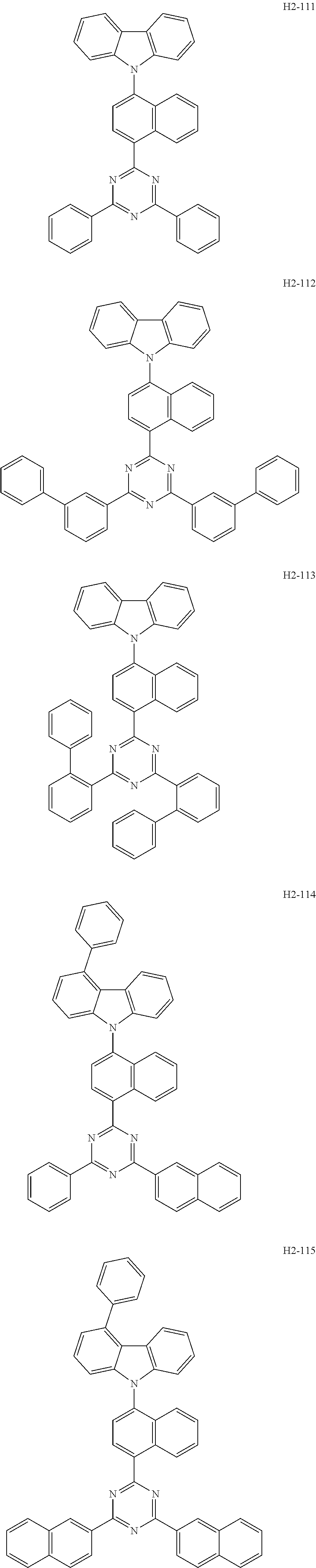 [SMILES: c1ccc(-c2nc(-c3ccccc3)nc(-c3ccc(-n4c5ccccc5c5ccccc54)c4ccccc34)n2)cc1 ; c1ccc(-c2cccc(-c3nc(-c4cccc(-c5ccccc5)c4)nc(-c4ccc(-n5c6ccccc6c6ccccc65)c5ccccc45)n3)c2)cc1 ; c1ccc(-c2ccccc2-c2nc(-c3ccccc3-c3ccccc3)nc(-c3ccc(-n4c5ccccc5c5ccccc54)c4ccccc34)n2)cc1 ; c1ccc(-c2nc(-c3ccc4ccccc4c3)nc(-c3ccc(-n4c5ccccc5c5c(-c6ccccc6)cccc54)c4ccccc34)n2)cc1 ; c1ccc(-c2cccc3c2c2ccccc2n3-c2ccc(-c3nc(-c4ccc5ccccc5c4)nc(-c4ccc5ccccc5c4)n3)c3ccccc23)cc1]

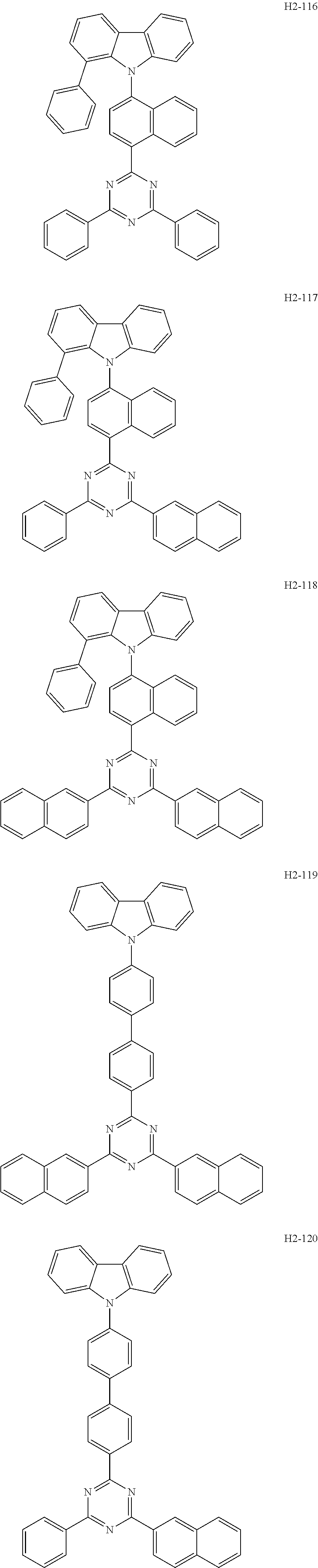 [SMILES: c1ccc(-c2nc(-c3ccccc3)nc(-c3ccc(-n4c5ccccc5c5cccc(-c6ccccc6)c54)c4ccccc34)n2)cc1 ; c1ccc(-c2nc(-c3ccc4ccccc4c3)nc(-c3ccc(-n4c5ccccc5c5cccc(-c6ccccc6)c54)c4ccccc34)n2)cc1 ; c1ccc(-c2cccc3c4ccccc4n(-c4ccc(-c5nc(-c6ccc7ccccc7c6)nc(-c6ccc7ccccc7c6)n5)c5ccccc45)c23)cc1 ; c1ccc2cc(-c3nc(-c4ccc(-c5ccc(-n6c7ccccc7c7ccccc76)cc5)cc4)nc(-c4ccc5ccccc5c4)n3)ccc2c1 ; c1ccc(-c2nc(-c3ccc(-c4ccc(-n5c6ccccc6c6ccccc65)cc4)cc3)nc(-c3ccc4ccccc4c3)n2)cc1]

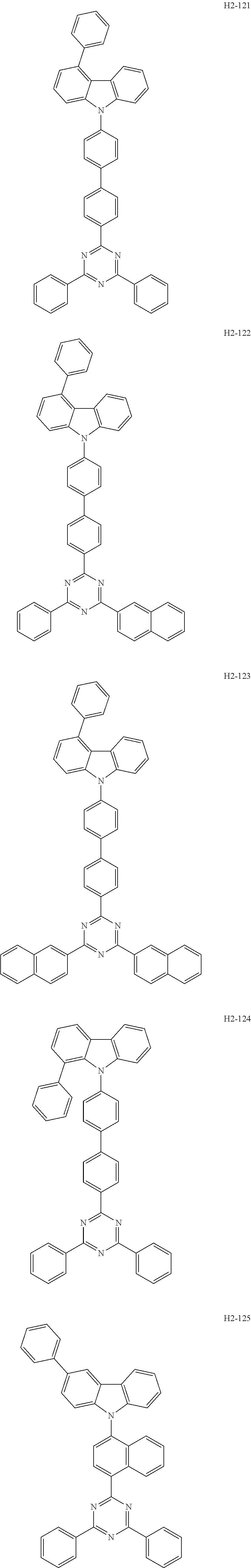 [SMILES: c1ccc(-c2nc(-c3ccccc3)nc(-c3ccc(-c4ccc(-n5c6ccccc6c6c(-c7ccccc7)cccc65)cc4)cc3)n2)cc1 ; c1ccc(-c2nc(-c3ccc(-c4ccc(-n5c6ccccc6c6c(-c7ccccc7)cccc65)cc4)cc3)nc(-c3ccc4ccccc4c3)n2)cc1 ; c1ccc(-c2cccc3c2c2ccccc2n3-c2ccc(-c3ccc(-c4nc(-c5ccc6ccccc6c5)nc(-c5ccc6ccccc6c5)n4)cc3)cc2)cc1 ; c1ccc(-c2nc(-c3ccccc3)nc(-c3ccc(-c4ccc(-n5c6ccccc6c6cccc(-c7ccccc7)c65)cc4)cc3)n2)cc1 ; c1ccc(-c2ccc3c(c2)c2ccccc2n3-c2ccc(-c3nc(-c4ccccc4)nc(-c4ccccc4)n3)c3ccccc23)cc1]

[chemical structure]

At least one of compounds H1-1 to H1-160 and at least one of compounds H2-1 to H2-139 may be combined and used in an organic electroluminescent device.

The compound represented by formula 1 of the present disclosure may be produced by a synthetic method known to one skilled in the art, and for example, according to the method disclosed in Korean Laid-open Patent Application No. 2017-0022865, etc., but is not limited thereto.

The compound represented by formula 2 of the present disclosure may be produced by a synthetic method known to one skilled in the art, and for example, according to the following reaction scheme, but is not limited thereto:

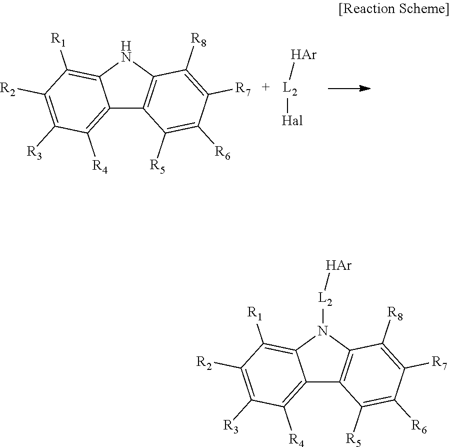

In the reaction scheme, R₁ to R₈, L₂, and HAr are as defined in formula 2, and Hal represents a halogen.

In addition, the non-deuterated derivative of the compound represented by formula 2 may be prepared by a known coupling or substitution reaction. The deuterated derivative may be prepared by a similar method using a deuterated precursor material, or more generally, treating a non-deuterated compound with a deuterated solvent, D6-benzene in the presence of a Lewis acid such as aluminum trichloride or ethyl aluminum chloride, an H/D exchange catalyst such as trifluoromethanesulfonic acid or trifluoromethanesulfonic acid-D, etc. Further, the degree of deuteration may be controlled by varying reaction conditions such as reaction temperature. For example, by controlling the reaction temperature and time, acid equivalent, etc., the degree of deuteration in formula 2 may be controlled.

The organic electroluminescent device according to the present disclosure may comprise an anode, a cathode, and at least one organic layer between the anode and cathode in which the organic layer may comprise a plurality of organic electroluminescent materials, including the compound represented by formula 1 as the first organic electroluminescent material, and the compound represented by formula 2 as the second organic electroluminescent material. According to one embodiment of the present disclosure, the organic electroluminescent device according to the present disclosure may comprise an anode, a cathode, and at least one light-emitting layer between the anode and cathode in which the light-emitting layer may comprise the compound represented by formula 1 and the compound represented by formula 2.

The light-emitting layer includes a host and a dopant, in which the host includes a plurality of host materials and the compound represented by formula 1 may be included as the first host compound of the plurality of host materials, and the compound represented by formula 2 may be included as the second host compound of the plurality of host materials. The weight ratio of the first host compound and the second host compound is about 1:99 to about 99:1, preferably about 10:90 to about 90:10, more preferably about 30:70 to about 70:30, even more preferably about 40:60 to about 60:40, and still more preferably about 50:50.

Herein, the light-emitting layer is a layer from which light is emitted, and may be a single layer or a multilayer of which two or more layers are stacked. All of the first host material and the second host material may be included in one layer, or the first host material and the second host material may be included in respective different light-emitting layers. According to one embodiment of the present disclosure, the doping concentration of the dopant compound with respect to the host compound in the light-emitting layer may be less than 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device of the present disclosure may further comprise an amine-based compound besides the plurality of host materials of the present disclosure as at least one of a hole injection material, a hole transport material, a hole auxiliary material, a light-emitting material, a light-emitting auxiliary material, and an electron blocking material. Further, according to one embodiment of the present disclosure, the organic electroluminescent device of the present disclosure may further comprise an azine-based compound besides the plurality of host materials of the present disclosure as at least one of an electron transport material, an electron injection material, an electron buffer material, and a hole blocking material.

The plurality of host materials according to the present disclosure may be used as light-emitting materials for a white organic light-emitting device. The white organic light-emitting device has been suggested to have various structures such as a parallel arrangement (side-by-side) method, a stacking method, or color conversion material (CCM) method, etc., according to the arrangement of R (red), G (green) or YG (yellowish green), B (blue) light-emitting units. In addition, the plurality of host materials according to an embodiment of the present disclosure may also be used in an organic electroluminescent device comprising a quantum dot (QD).

In the organic electroluminescent device of the present disclosure, a hole injection layer, a hole transport layer, an electron blocking layer, or a combination thereof can be used between the anode and the light-emitting layer. The hole injection layer may be multilayered in order to lower the hole injection barrier (or hole injection voltage) from the anode to the hole transport layer or the electron blocking layer, wherein each of the multilayers may use two compounds simultaneously. Further, the hole injection layer may be doped with a p-dopant. The electron blocking layer may be placed between the hole transport layer (or hole injection layer) and the light-emitting layer, and may block overflowing electrons from the light-emitting layer and confine the excitons in the light-emitting layer to prevent light leakage.

The hole transport layer or the electron blocking layer may also be multilayered, wherein each of the multilayers may use a plurality of compounds.

An electron buffer layer, a hole blocking layer, an electron transport layer, an electron injection layer, or a combination thereof can be used between the light-emitting layer and the cathode. The electron buffer layer may be multilayered in order to control the electron injection and improve the interfacial properties between the light-emitting layer and the electron injection layer, wherein each of the multilayers may use two compounds simultaneously. The hole blocking layer or the electron transport layer may also be multilayered, wherein each of the multilayers may use a plurality of compounds. In addition, the electron injection layer may be doped with an n-dopant.

The dopant comprised in the organic electroluminescent device of the present disclosure may be at least one phosphorescent or fluorescent dopant, and is preferably at least one phosphorescent dopant. The phosphorescent dopant material applied to the organic electroluminescent device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise a compound represented by the following formula 101, but is not limited thereto.

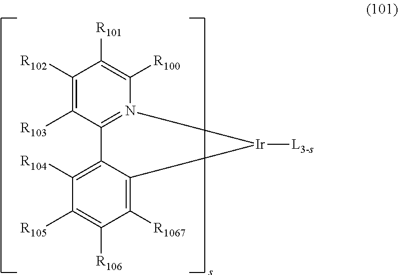

In formula 101, L is selected from the following structures 1 and 2:

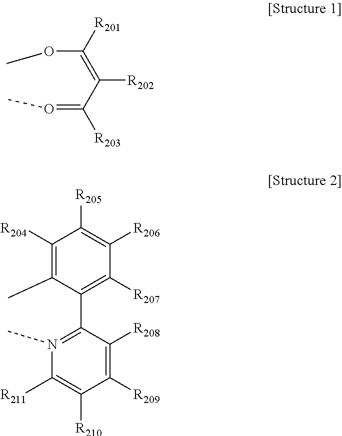

R₁₀₀ to R₁₀₃ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30) aryl, a cyano, a substituted or unsubstituted (3- to 30-membered) heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline ring, together with pyridine;
R₁₀₄ to R₁₀₇ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30) aryl, a substituted or unsubstituted (3- to 30-membered) heteroaryl, a cyano, or a substituted or unsubstituted (C1-C30)alkoxy; or may be linked to an adjacent substituent to form a ring(s), e.g., a substituted or unsubstituted, naphthyl, fluorene, dibenzothiophene, dibenzofuran, indenopyridine, benzofuropyridine, or benzothienopyridine ring, together with benzene;
R₂₀₁ to R₂₁₁ each independently represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30) aryl; or may be linked to an adjacent substituent to form a ring(s); and
s represents an integer of 1 to 3.

The specific examples of the dopant compound are as follows, but are not limited thereto.

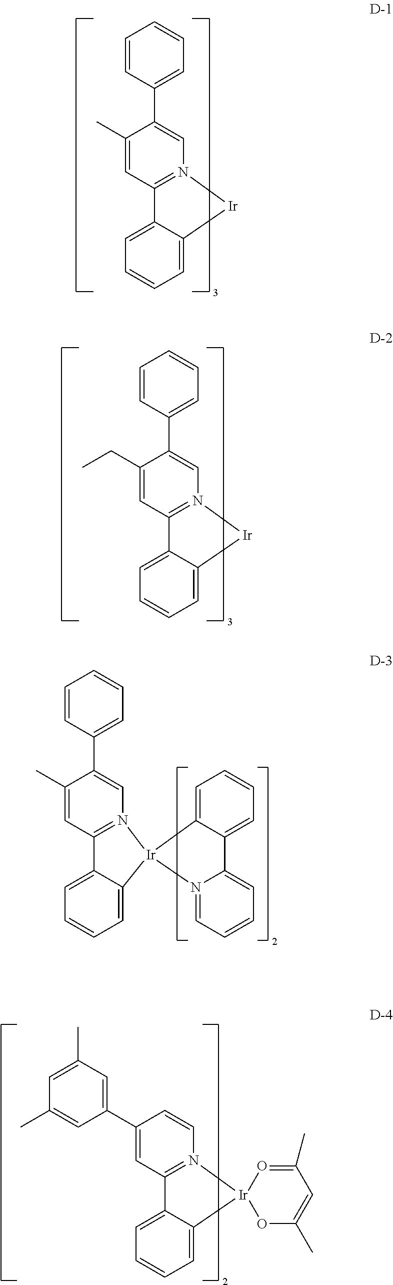

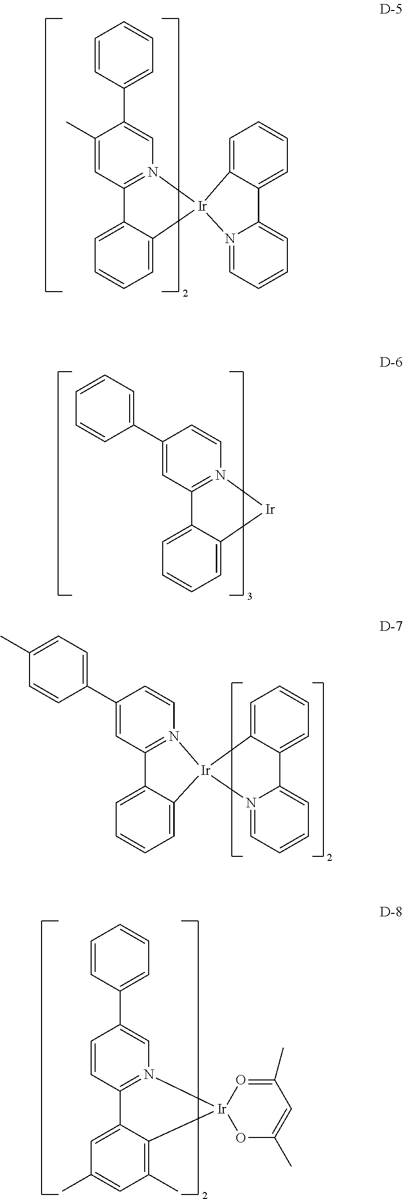

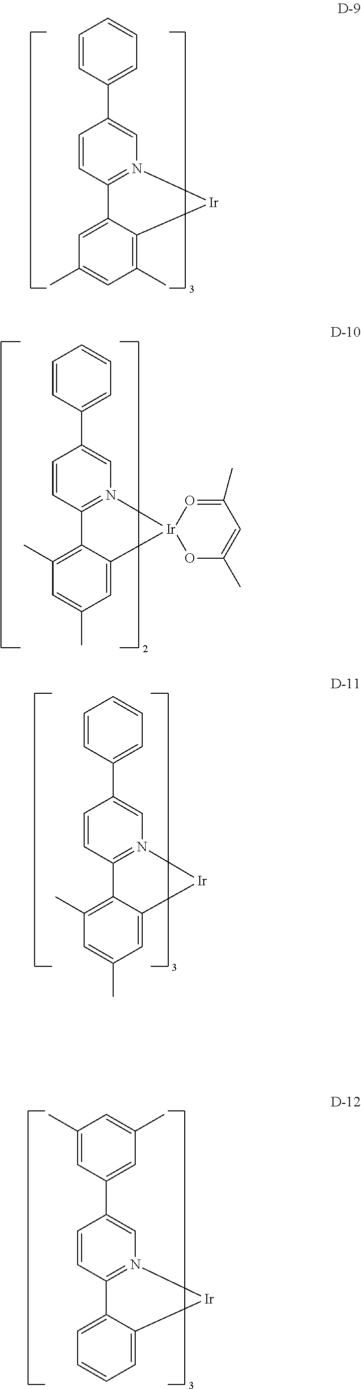

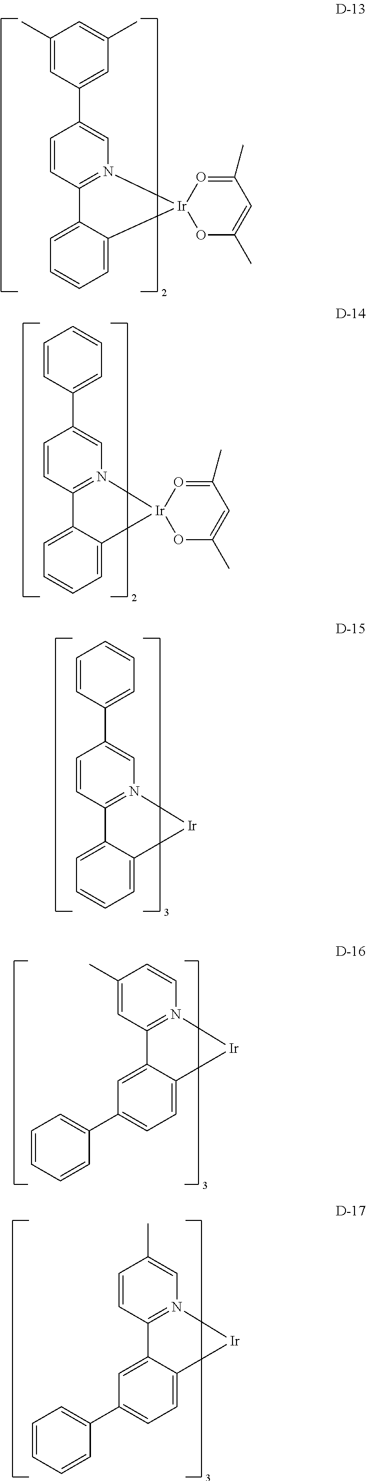

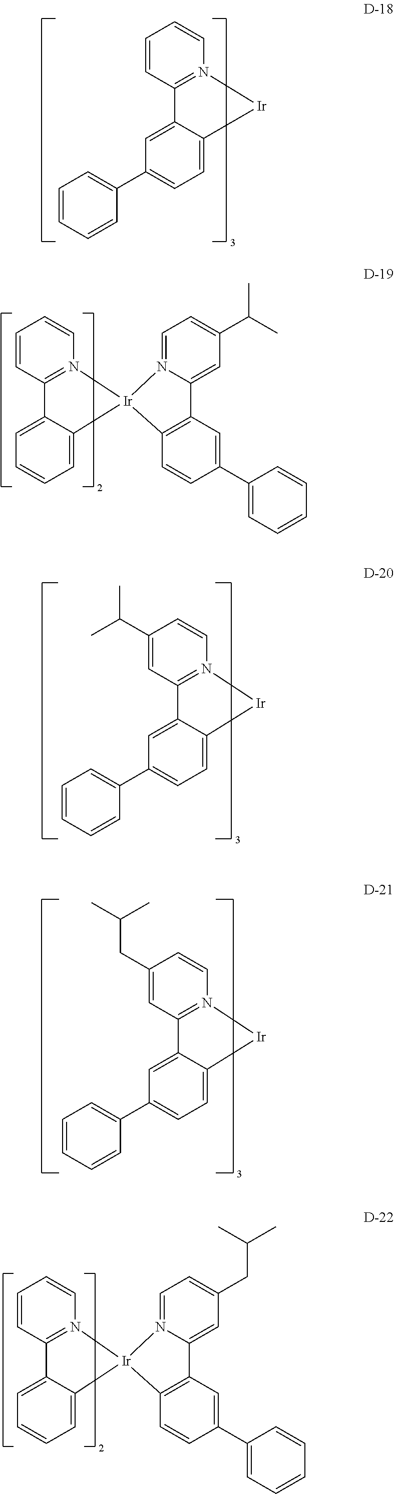

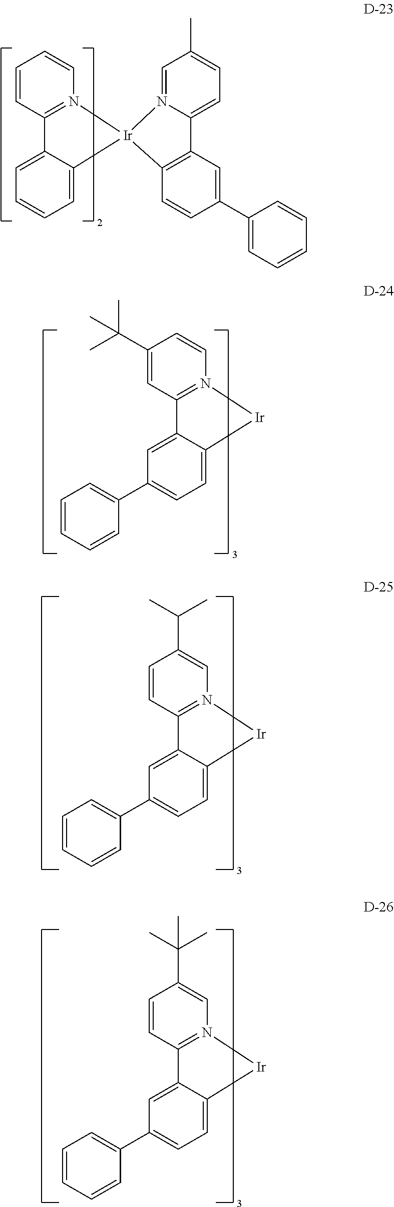

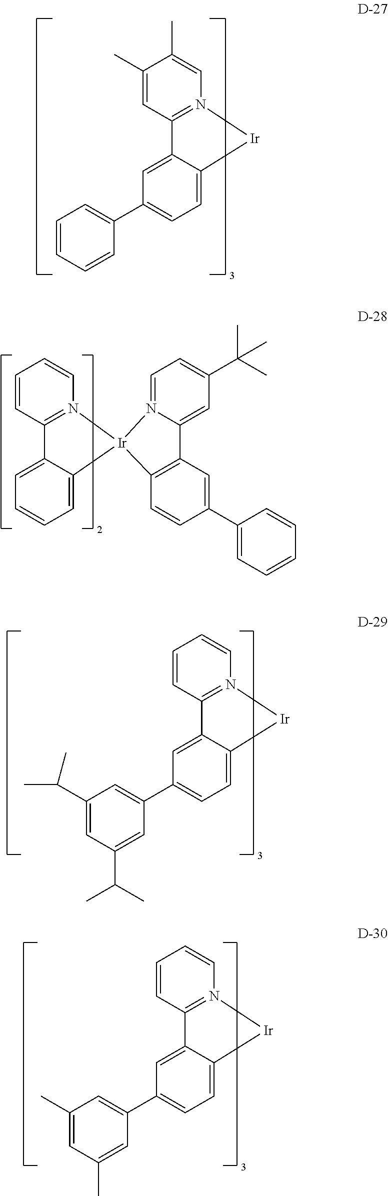

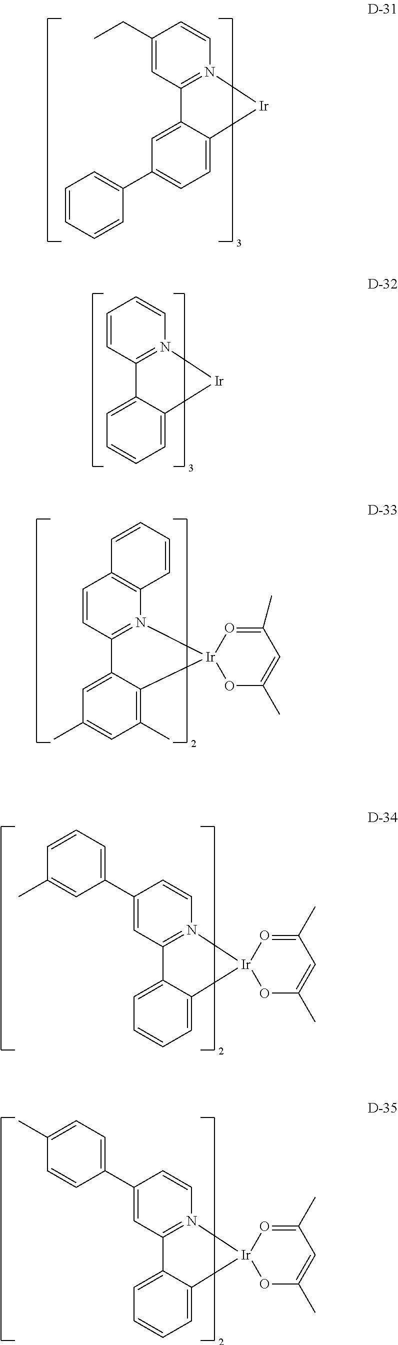

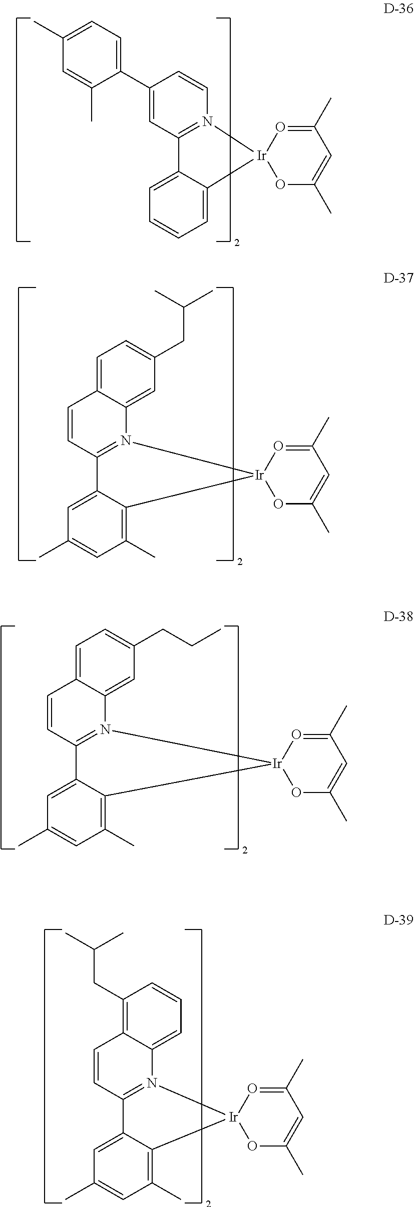

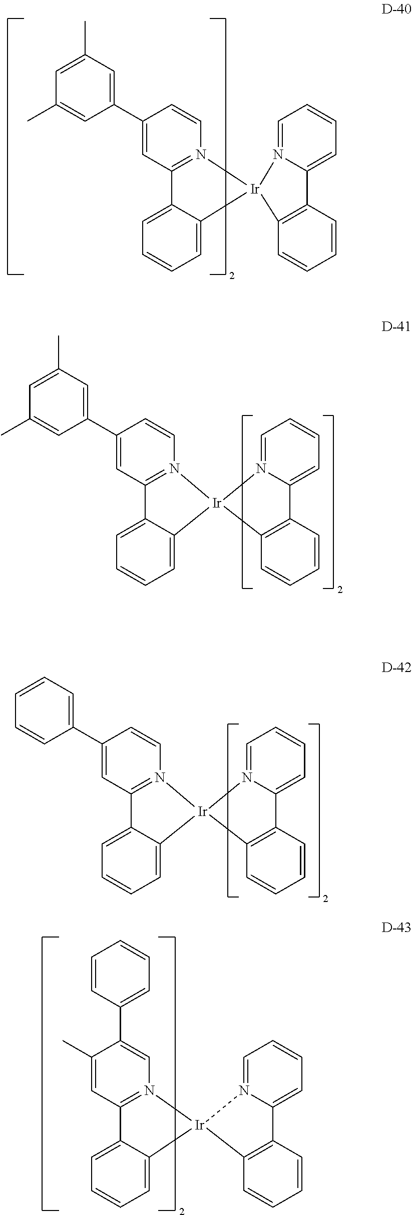

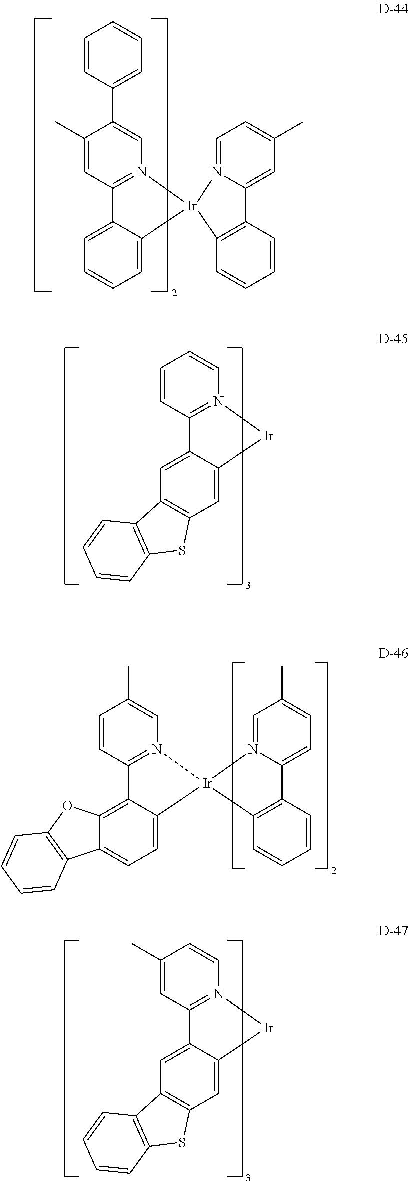

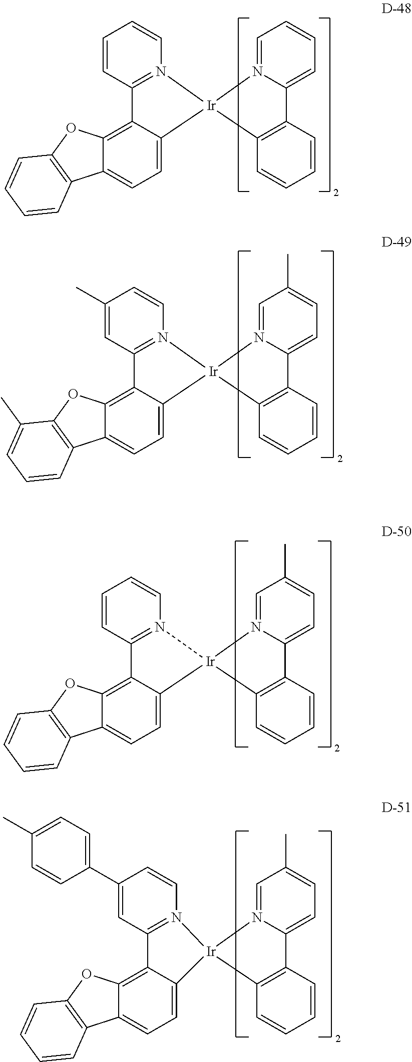

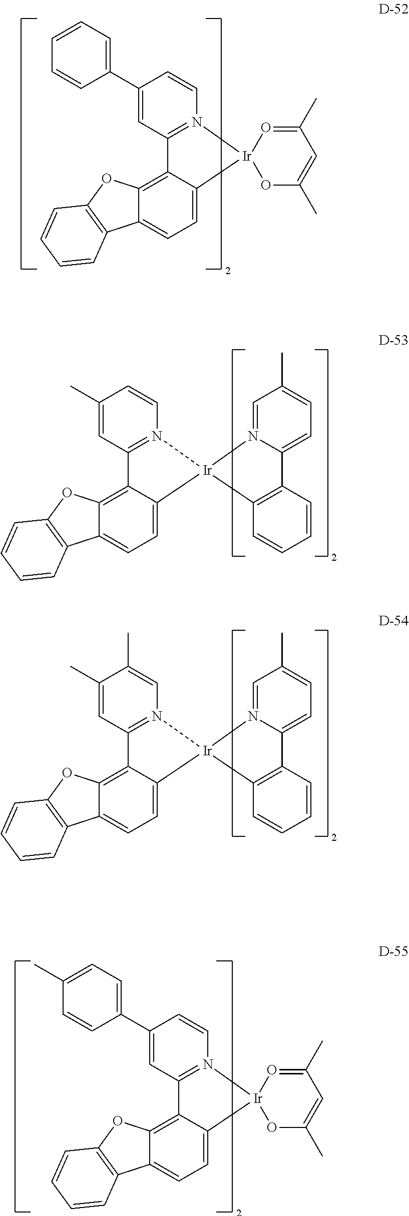

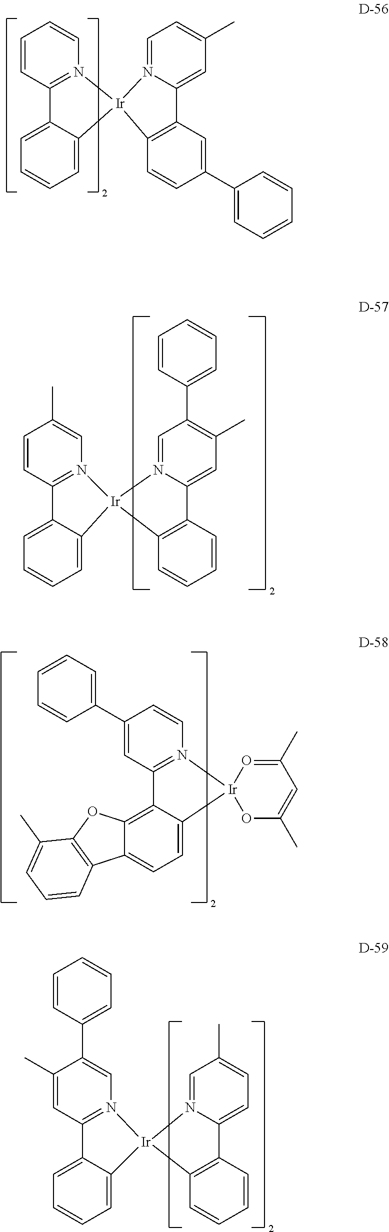

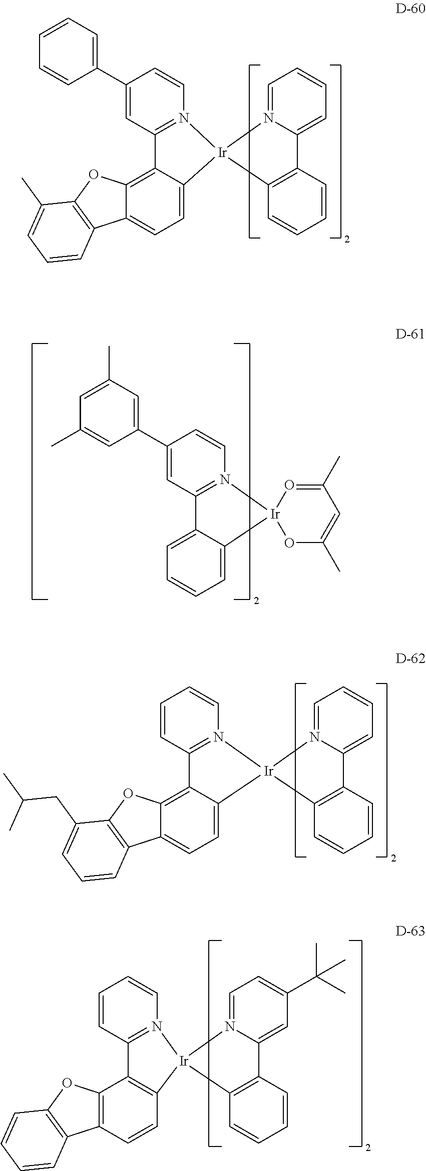

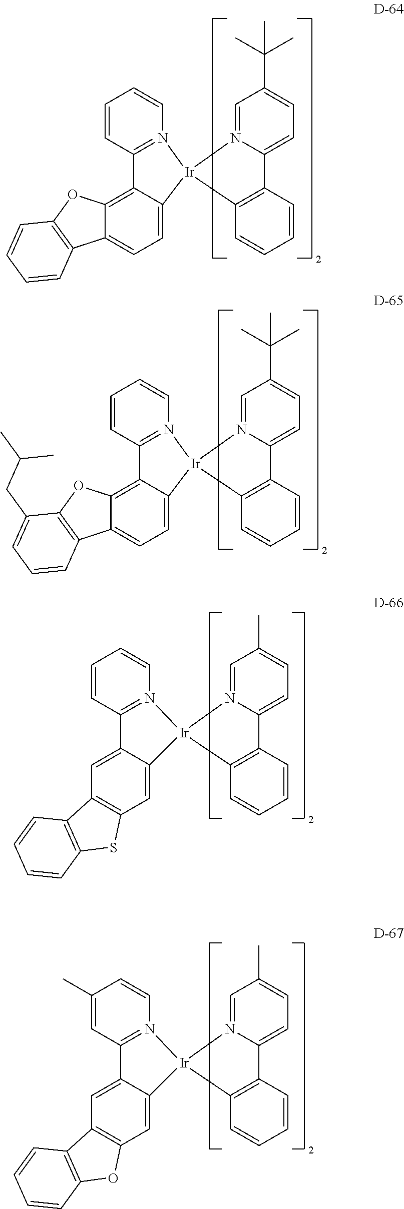

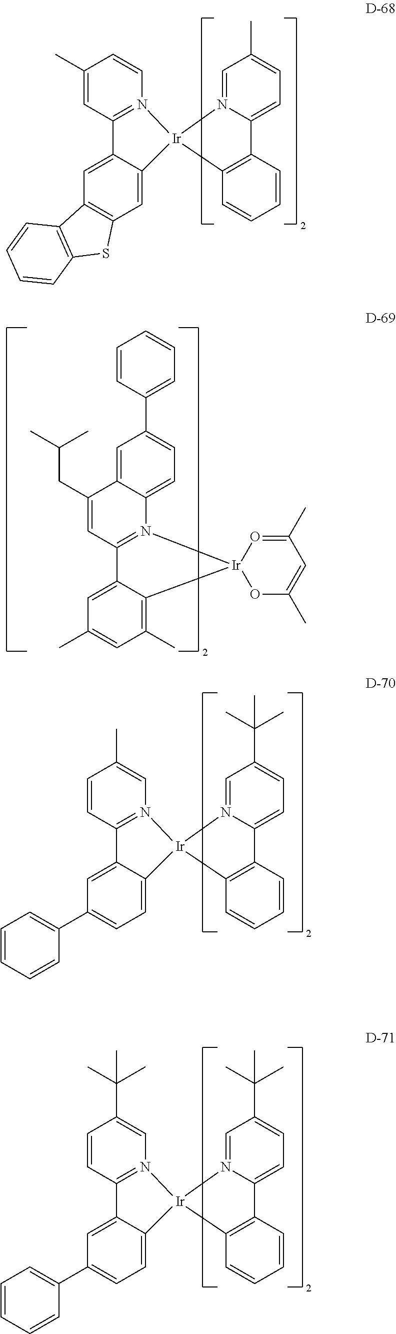

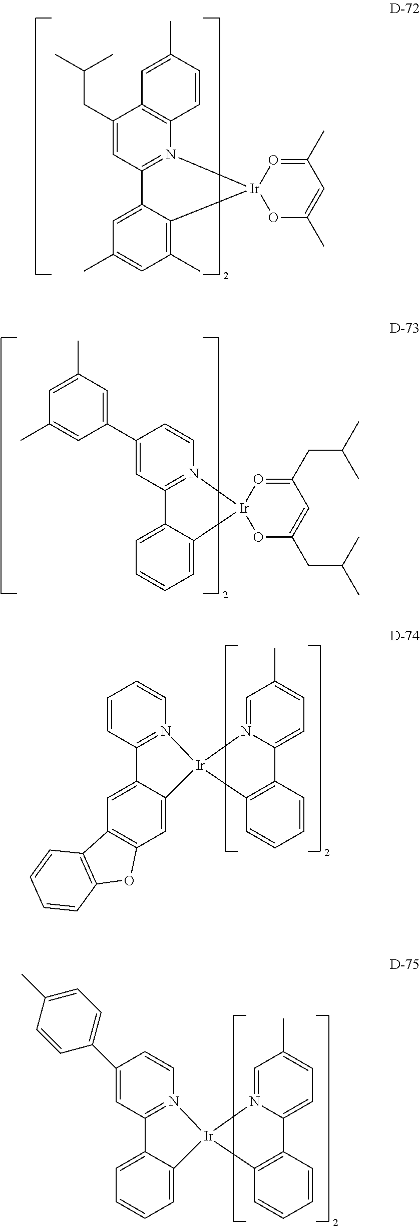

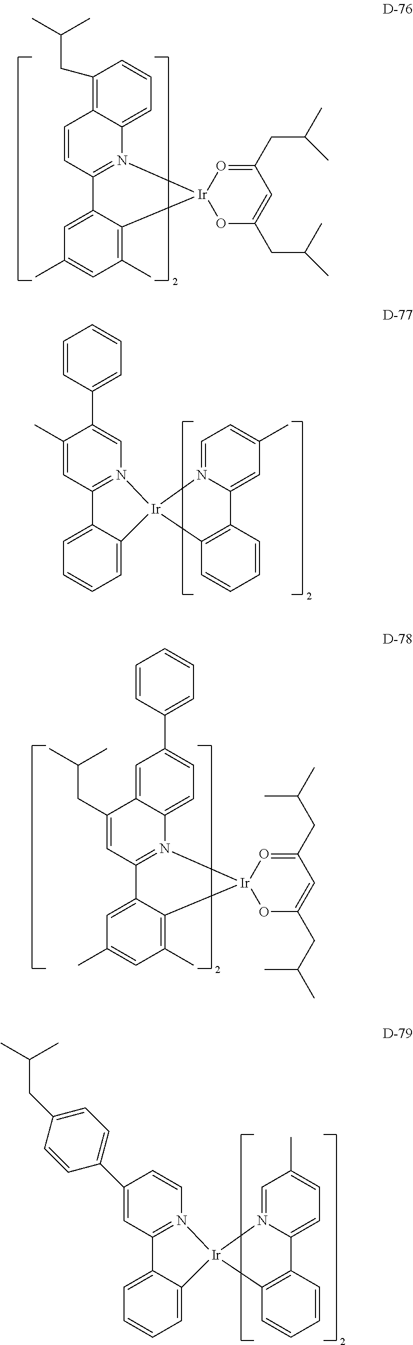

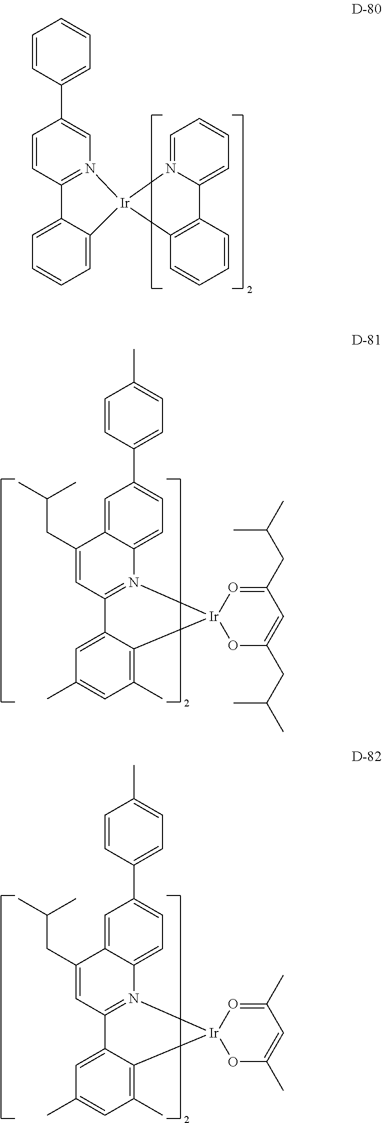

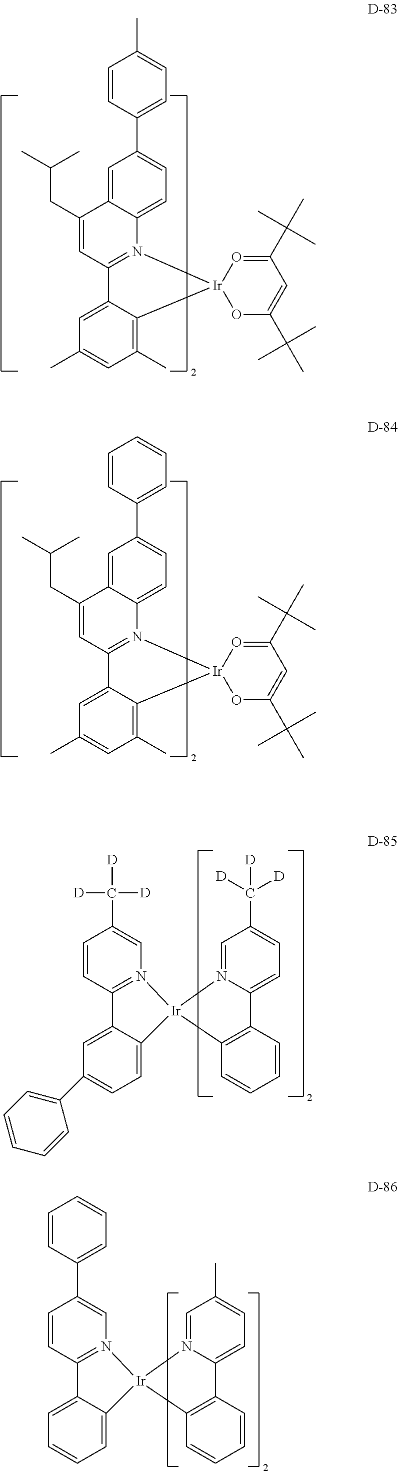

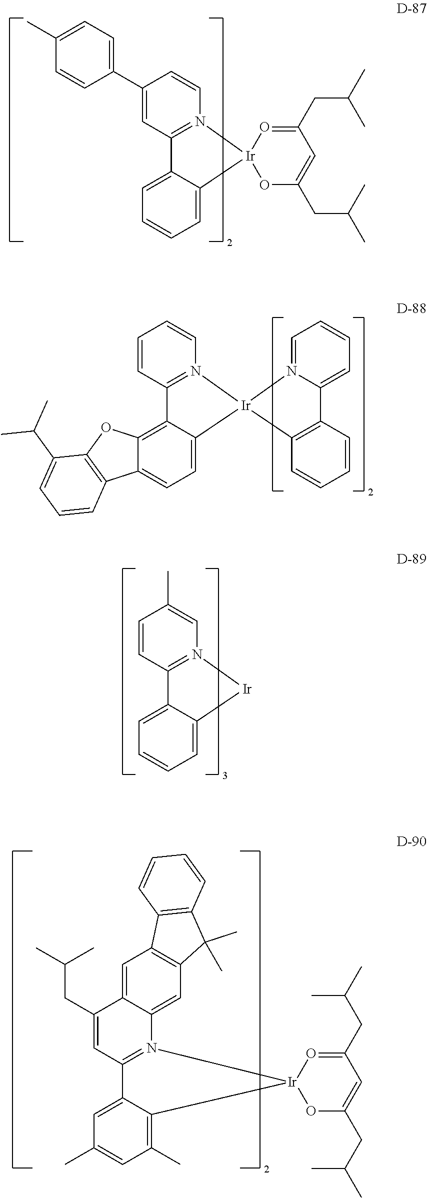

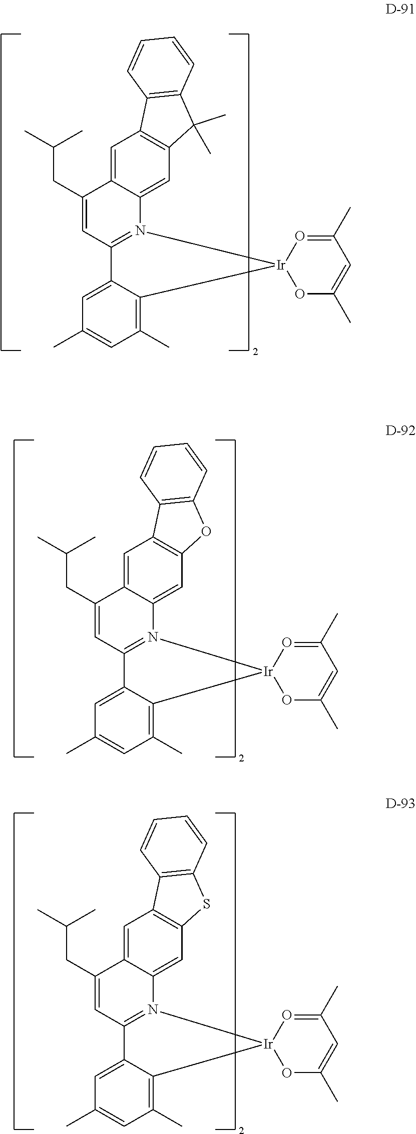

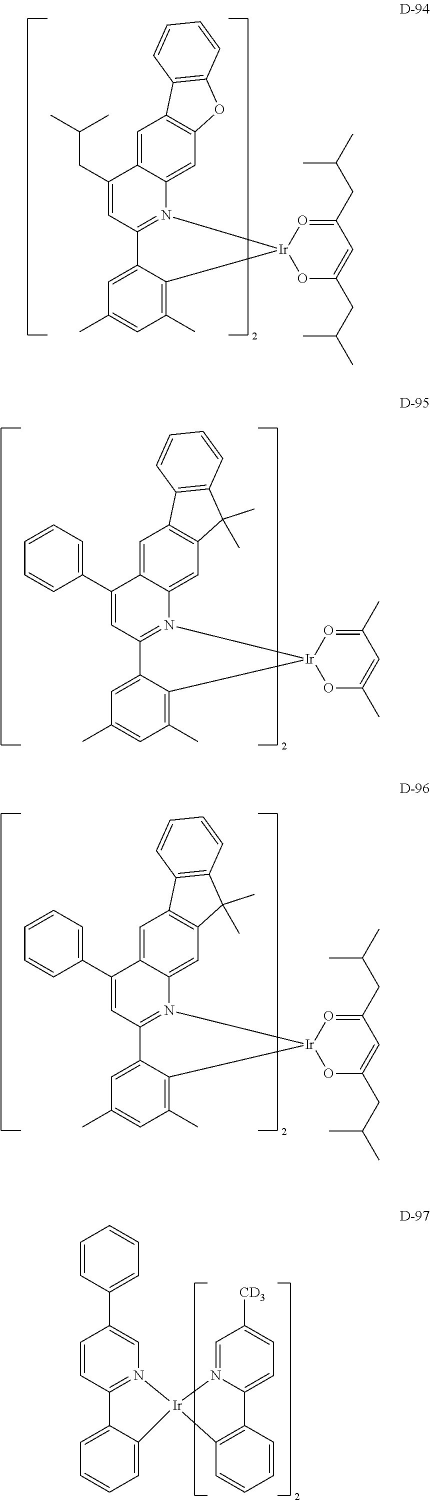

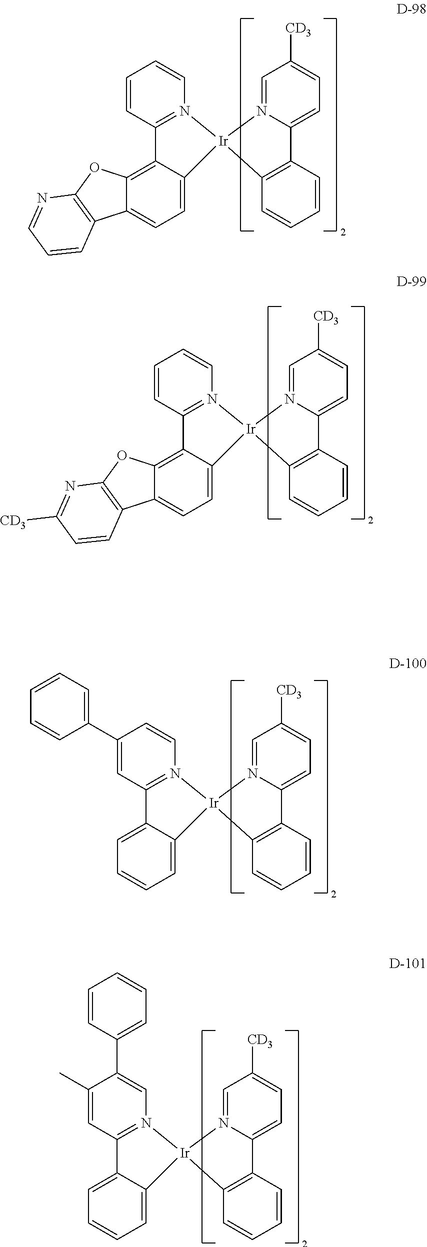

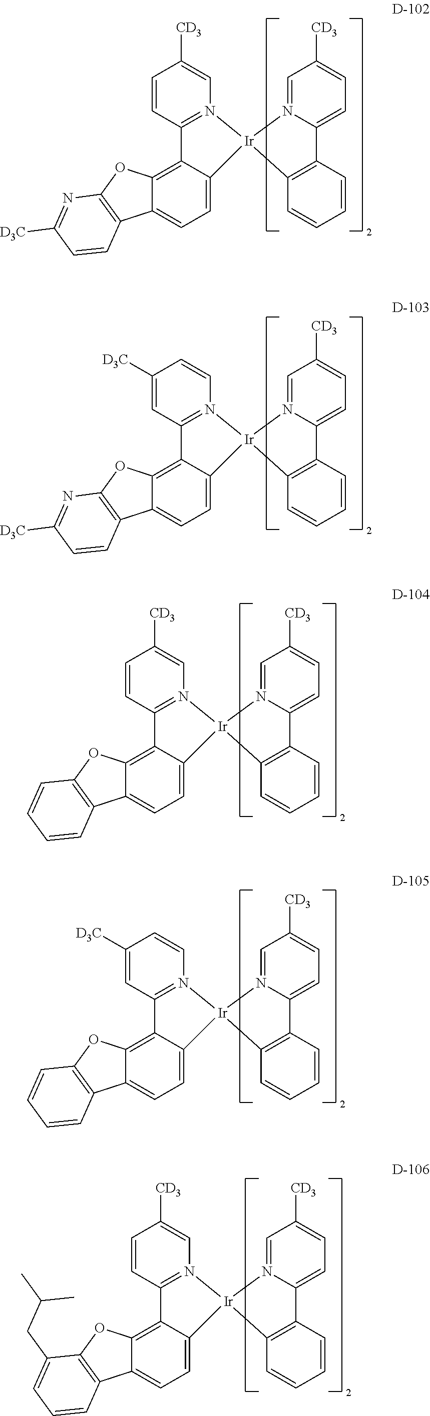

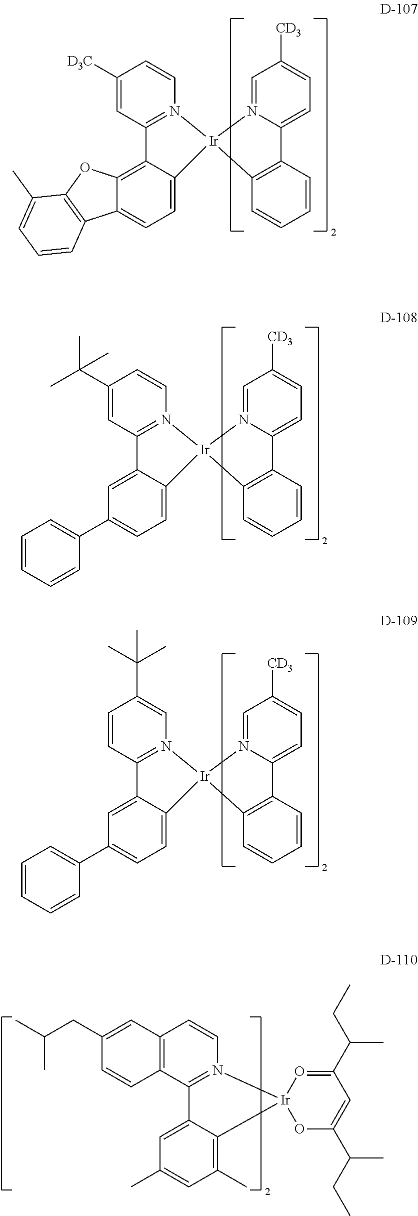

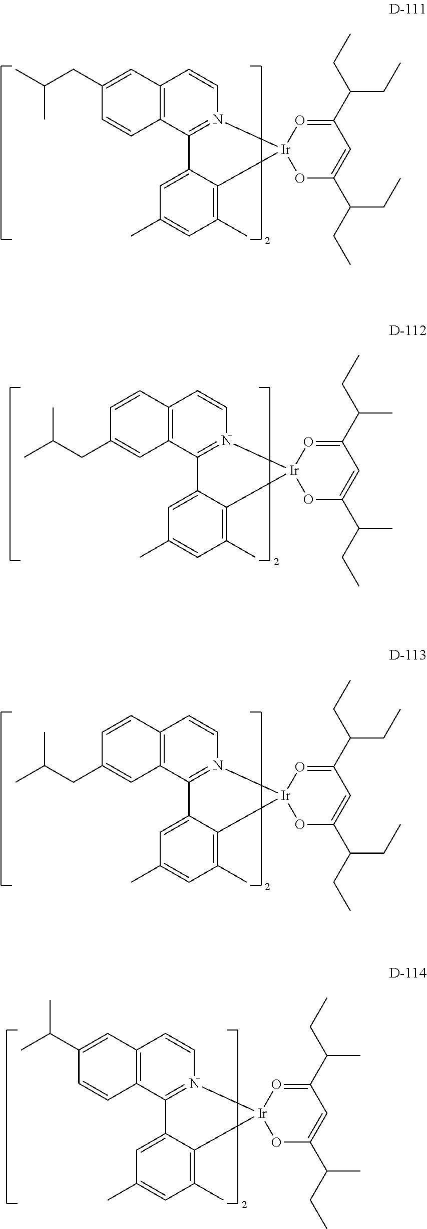

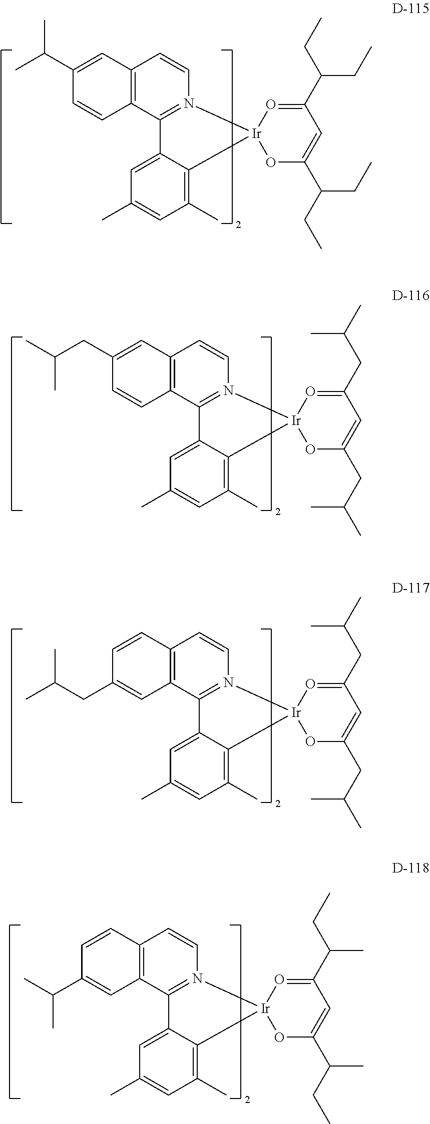

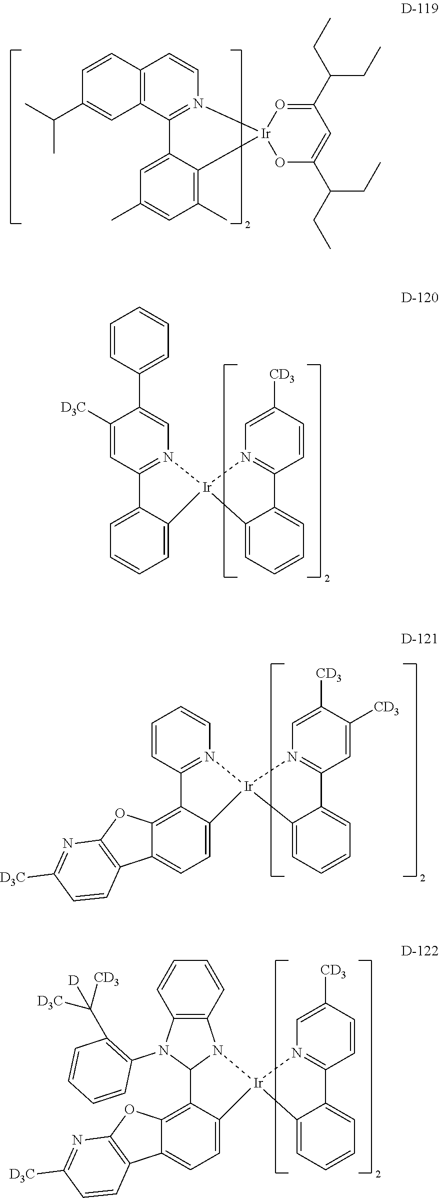

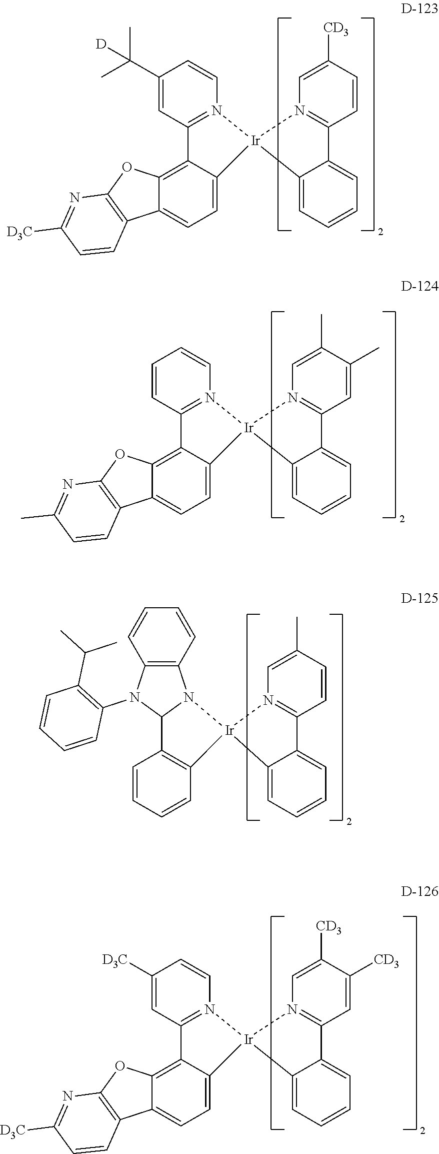

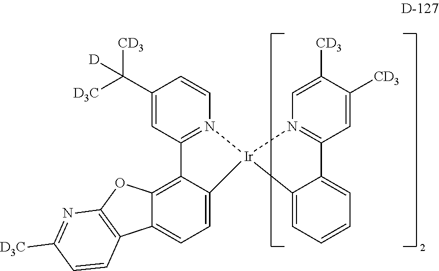

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing the materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

The first and the second host compounds of the present disclosure may be film-formed by the above-listed methods, commonly by a co-evaporation process or a mixture-evaporation process. The co-evaporation method is a mixed deposition method in which two or more materials are placed in a respective individual crucible source and a current is applied to both cells at the same time to evaporate the materials. The mixture-evaporation method is a mixed deposition method in which two or more materials are mixed in one crucible source before evaporating them, and a current is applied to the cell to evaporate the materials. Further, if the first and the second host compounds are present in the same layer or different layers in an organic electroluminescent device, the two host compounds may individually form films. For example, the second host compound may be deposited after depositing the first host compound.

The present disclosure may provide a display system by using the plurality of host materials including the compound represented by formula 1 and the compound represented by formula 2. That is, by using the plurality of host materials of the present disclosure, it is possible to manufacture a display system or a lighting system. Specifically, by using the plurality of host materials of the present disclosure, a display system, for example, for white organic light emitting devices, smart phones, tablets, notebooks, PCs, TVs, or cars; or a lighting system, for example an outdoor or indoor lighting system, can be produced.

Hereinafter, the preparation method of the compounds according to the present disclosure and the properties thereof, and the properties of an organic electroluminescent device comprising the plurality of host materials of the present disclosure will be explained in detail with reference to the representative compounds of the present disclosure. However, the present disclosure is not limited by the following examples.

### Synthesis Example 1: Preparation of Compound H1-147

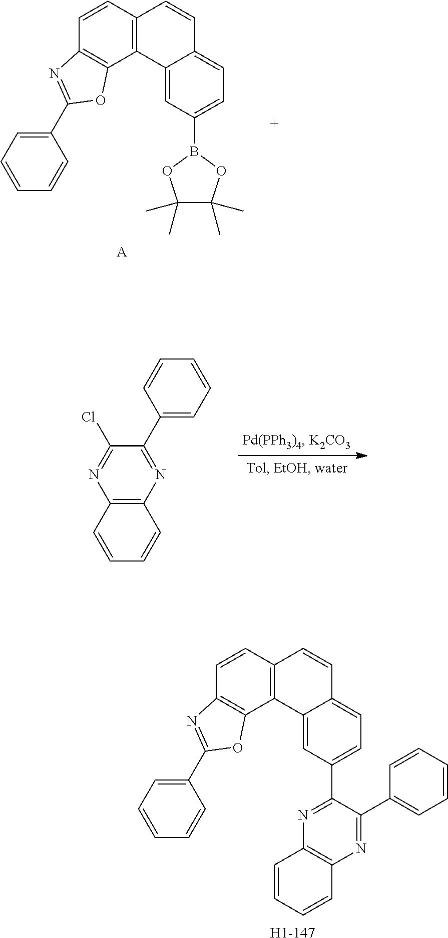

Compound A (CAS: 2085325-18-2, 4.0 g, 9.5 mmol), 2-chloro-3-phenylquinoxaline (2.8 g, 11.4 mmol), tetrakis(triphenylphosphine)palladium(0) (Pd(PPh₃)₄) (0.5 g, 0.5 mmol), potassium carbonate (K₂CO₃) (2.0 g, 19 mmol), toluene (30 mL), EtOH (7 mL), and water (10 mL) were added into a reaction vessel and stirred under reflux for one day. After completion of the reaction, followed by cooling at room temperature, the reaction mixture was filtered with a celite filter with methylene chloride (MC) and then distilled under reduced pressure. The residue was separated by column chromatography with methylene chloride/hexane (MC/Hex) to obtain compound H1-147 (2.7 g, yield: 57%).

| Compound | MW | M.P. |
|---|---|---|
| H1-147 | 499.6 | 266° C. |

### Synthesis Example 2: Preparation of Compound H1-146

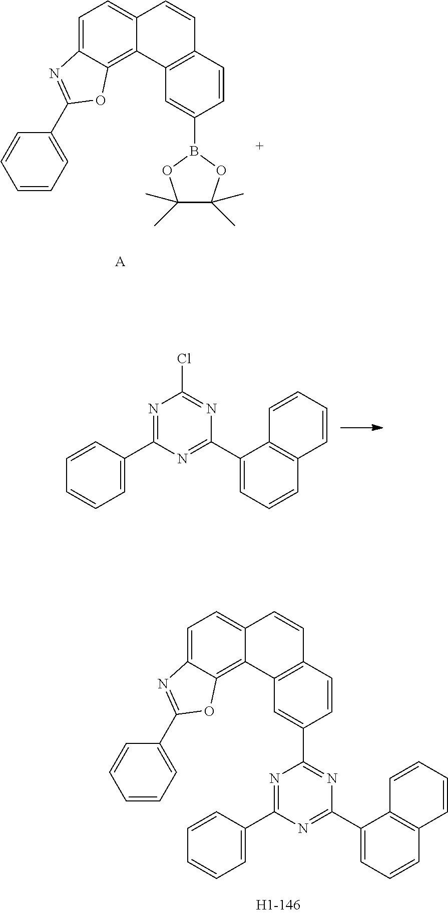

Compound A (23.8 g, 56.6 mmol), 2-chloro-4-(naphthalen-1-yl)-6-phenyl-1,3,5-triazine (15.0 g, 47.2 mmol), Pd(PPh₃)₄ (2.72 g, 2.36 mmol), K₂CO₃ (16.3 g, 118 mmol), toluene (240 mL), EtOH (60 mL), and purified water (60 mL) were added into a reaction vessel and stirred under reflux for 2 hours. After completion of the reaction, the reaction mixture was cooled to room temperature and the organic layer was separated by a silica filter. The organic layer was distilled under reduced pressure, and recrystallized with toluene to obtain compound H1-146 (13.8 g, yield: 51%).

| Compound | MW | M.P. |
|---|---|---|
| H1-146 | 576.6 | 231° C. |

### Synthesis Example 3: Preparation of Compound H1-157

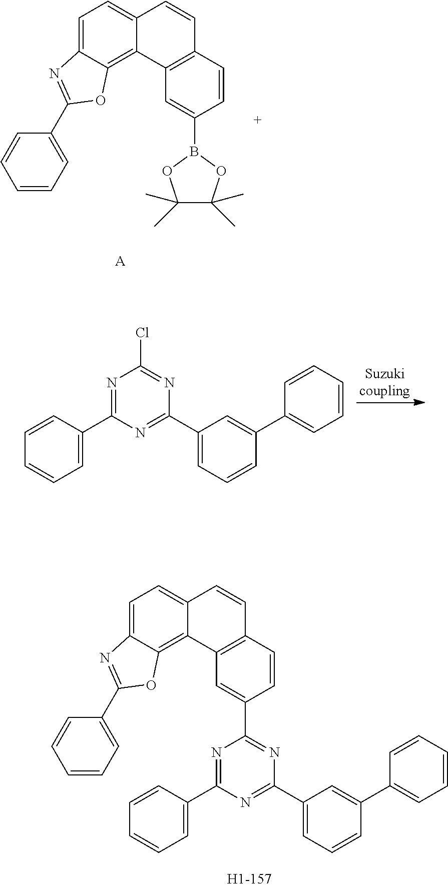

Compound A (4.0 g, 9.5 mmol), 2-([1,1′-biphenyl]-3-yl)-4-chloro-6-phenyl-1,3,5-triazine (3.9 g, 11.4 mmol), Pd(PPh₃)=₄ (0.5 g, 0.5 mmol), K₂CO₃ (2.6 g, 19 mmol), toluene (30 mL), EtOH (7 mL), and purified water (10 mL) were added into a reaction vessel and stirred under reflux for 6 hours. After completion of the reaction, followed by cooling to room temperature, the reaction mixture was stirred at room temperature, and then methanol (MeOH) was added thereto. The resulting solid was filtered under reduced pressure, and separated by column chromatography with MC to obtain compound H1-157 (4.6 g, yield: 80%).

| Compound | MW | M.P. |
|---|---|---|
| H1-157 | 602.7 | 227° C. |

### Synthesis Example 4: Preparation of Compound H1-145

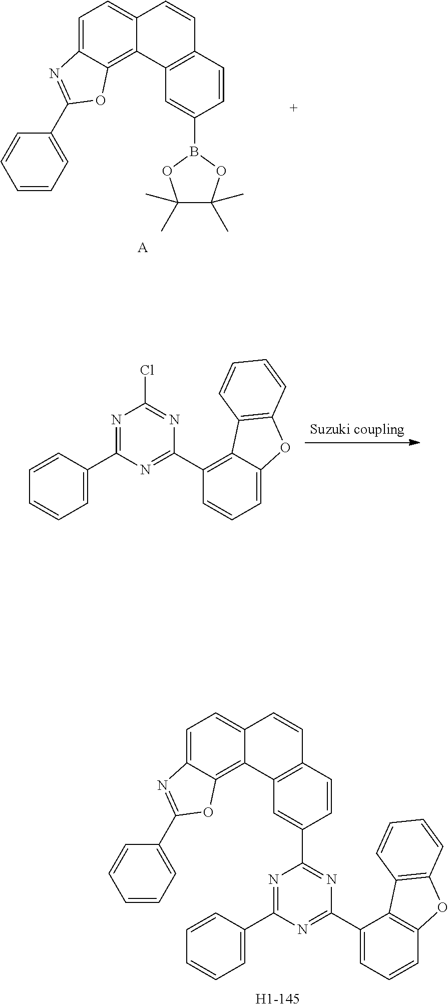

Compound A (3.0 g, 7.1 mmol), 2-chloro-4-(dibenzo[b,d]furan-1-yl)-6-phenyl-1,3,5-triazine (3.4 g, 9.26 mmol), Pd(PPh₃)₄ (0.4 g, 0.36 mmol), K₂CO₃ (2.0 g, 14 mmol), toluene (36 mL), EtOH (8 mL), and purified water (12 mL) were added into a reaction vessel and stirred under reflux for 6 hours. After completion of the reaction, followed by cooling to room temperature, the reaction mixture was stirred at room temperature, and then methanol (MeOH) was added thereto. The resulting solid was filtered under reduced pressure, and separated by column chromatography with MC to obtain compound H1-145 (3.3 g, yield: 75%).

| Compound | MW | M.P. |
|---|---|---|
| H1-145 | 616.7 | 282° C. |

### Synthesis Example 5: Preparation of Compound H1-156

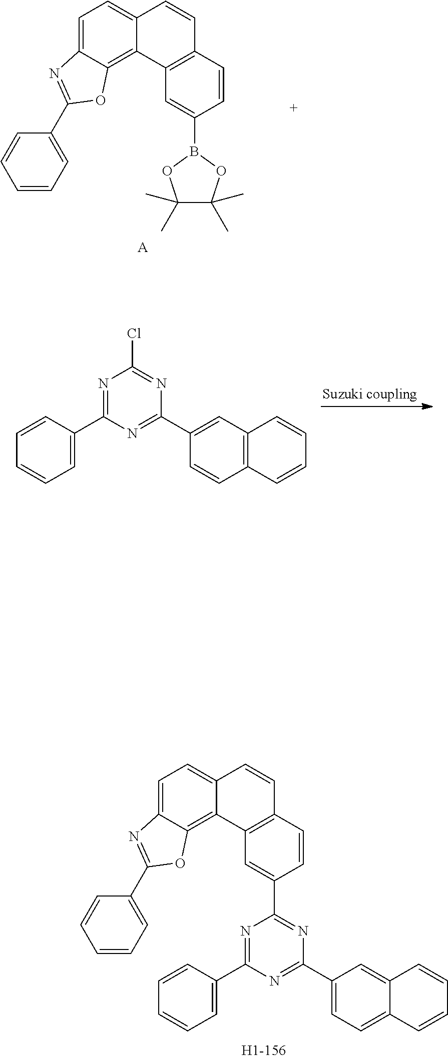

Compound A (4.0 g, 9.5 mmol), 2-chloro-4-(naphthalen-2-yl)-6-phenyl-1,3,5-triazine (3.6 g, 11.4 mmol), Pd(PPh₃)₄ (0.5 g, 0.5 mmol), K₂CO₃ (2.6 g, 19 mmol), toluene (30 mL), EtOH (7 mL), and purified water (10 mL) were added into a reaction vessel and stirred under reflux for 4 hours. After completion of the reaction, followed by cooling to a room temperature, the reaction mixture was stirred at room temperature, and then methanol (MeOH) was added thereto. The resulting solid was filtered under reduced pressure, and separated by column chromatography with MC to obtain compound H1-156 (3.45 g, yield: 63%).

| Compound | MW | M.P. |
|---|---|---|
| H1-156 | 576.6 | 268° C. |

### Synthesis Example 6: Preparation of Compound H1-51

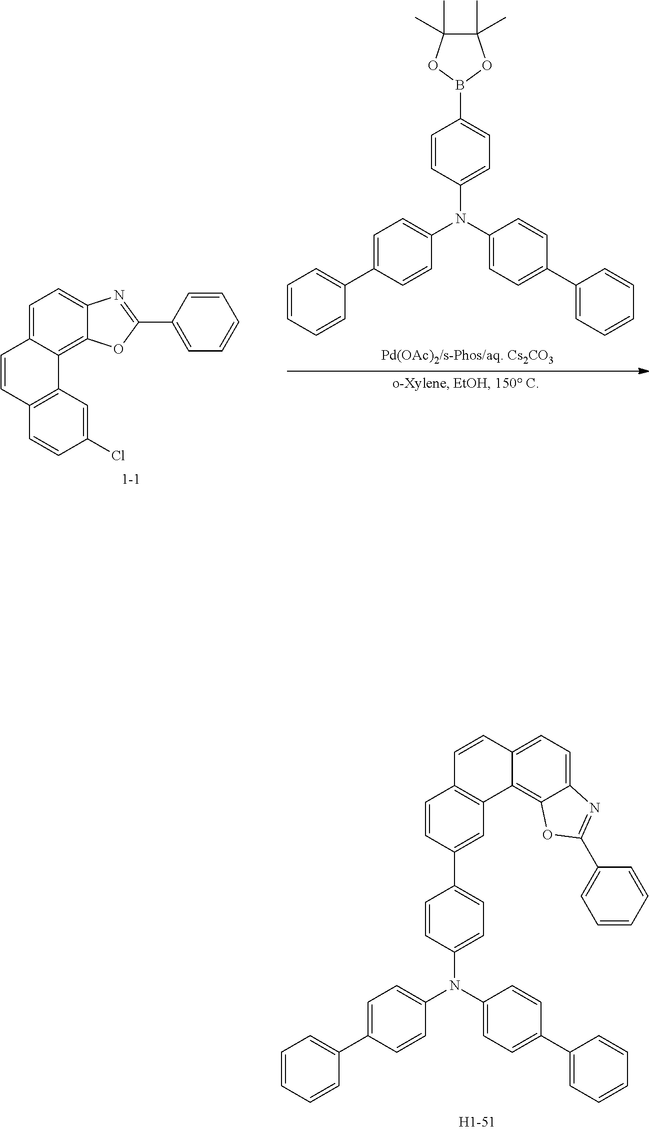 [SMILES: Clc1ccc2ccc3ccc4nc(-c5ccccc5)oc4c3c2c1 ; CC1(C)OB(c2ccc(N(c3ccc(-c4ccccc4)cc3)c3ccc(-c4ccccc4)cc3)cc2)OC1(C)C ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1]

Compound 1-1 (4 g, 12 mmol), bis(biphenyl-4-yl) [4-(4,4,5,5-tetramethyl-[1,3,2]-dioxaborolan-2-yl)phenyl]amine (6.8 g, 13 mmol), palladium (II) acetate (Pd(OAc)₂) (0.3 g, 1 mmol), 2-dicyclohexylphosphino-2′,6′-dimethoxybiphenyl (s-Phos) (0.9 g, 2 mmol), cesium carbonate (Cs₂CO₃) (11.5 g, 35 mmol), o-xylene (60 mL), ethanol (EtOH) (15 mL), and distilled water (15 mL) were added into a reaction vessel and stirred under reflux for 3 hours at 150° C. After completion of the reaction, the reaction mixture was washed with distilled water and then the organic layer was extracted with ethyl acetate. After drying the extracted organic layer with magnesium sulfate, the solvent was removed with a rotary evaporator and then purified with column chromatography to obtain compound H1-51 (2.2 g, yield: 27%).

| Compound | MW | UV | PL | M.P. |
|---|---|---|---|---|
| H1-51 | 690.85 | 406 nm | 427 nm | 271° C. |

### Synthesis Example 7: Preparation of Compound H1-80

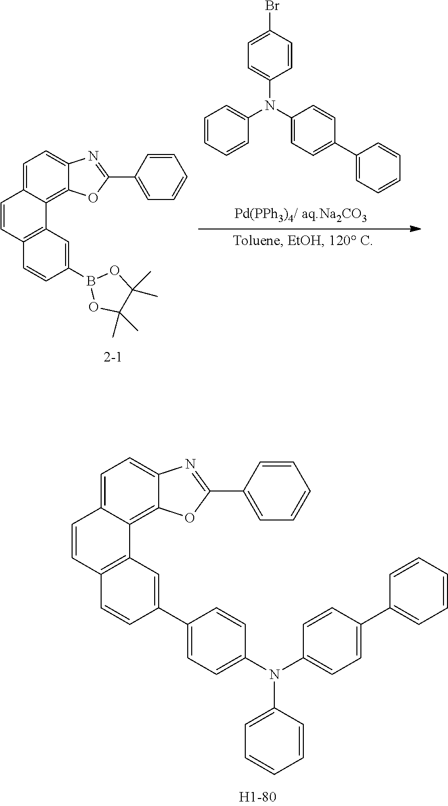

Compound 2-1 (4.8 g, 11.34 mmol), N-(4-bromophenyl)-N-phenyl-[1,1′-biphenyl]-4-amine (5 g, 12.47 mmol), Pd(PPh₃)₄ (0.4 g, 0.34 mmol), sodium carbonate (Na₂CO₃) (3.0 g, 28.35 mmol), toluene (57 mL), EtOH (14 mL), and distilled water (14 mL) were added into a reaction vessel and stirred for 4 hours at 120° C. After completion of the reaction, the mixture was added dropwise to methanol, and then the resulting solid was filtered. The resulting solid was purified by recrystallization with column chromatography to obtain compound H1-80 (1.4 g, yield: 20.0%).

| Compound | MW | M.P. |
|---|---|---|
| H1-80 | 614.73 | 230° C. |

### Synthesis Example 8: Preparation of Compound H1-158

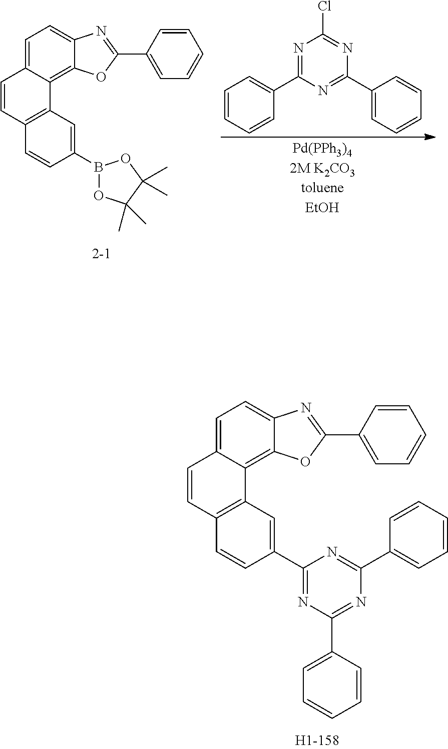

Compound 2-1 (10 g, 23.7 mmol), 2-chloro-4,6-diphenyltriazine (CAS: 3842-55-5, 5.8 g, 21.6 mmol), Pd(PPh₃)₄ (1.2 g, 1.0 mmol), potassium carbonate (K₂CO₃) (7.5 g, 59 mmol), toluene (90 mL), ethanol (30 mL), and distilled water (30 mL) were added into a reaction vessel and stirred for 4 hours at 120° C. After completion of the reaction, the mixture was added dropwise to methanol, and then the resulting solid was filtered. The resulting solid was purified by recrystallization by column chromatography to obtain compound H1-158 (5.7 g, yield: 50%).

| Compound | MW | UV | PL | M.P. |
|---|---|---|---|---|
| H1-158 | 526.18 | 290 nm | 427 nm | 291° C. |

### Synthesis Example 9: Preparation of Compound H1-102

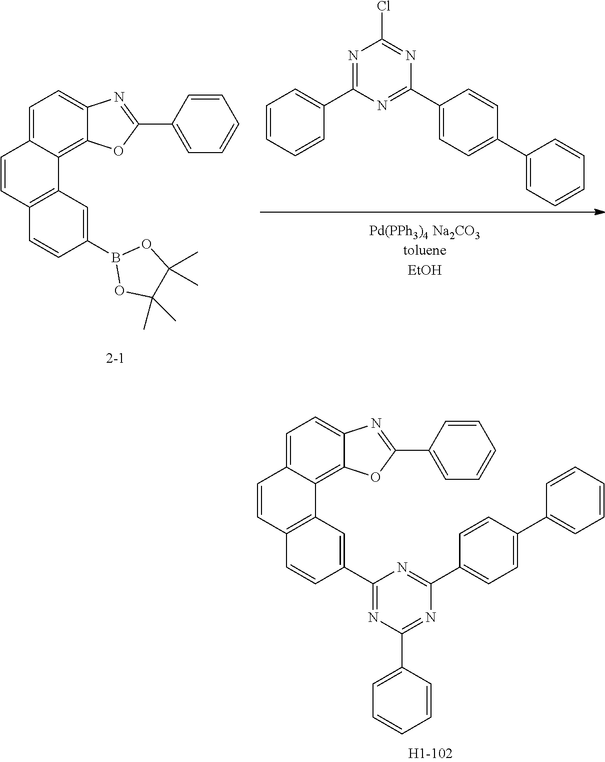

Compound 2-1 (3.48 g, 8.3 mmol), 2-([1,1′-biphenyl]-4-yl)-4-chloro-6-phenyl-1,3,5-triazine (CAS: 1472062-94-4, 3.53 g, 9.1 mmol), Pd(PPh₃)₄ (0.48 g, 0.41 mmol), sodium carbonate (2.2 g, 20.7 mmol), toluene (28 mL), ethanol (7 mL), and distilled water (7 mL) were added into a reaction vessel and stirred for 5 hours at 120° C. After completion of the reaction, the mixture was added dropwise to methanol, and then the resulting solid was filtered. The resulting solid was purified by recrystallization by column chromatography to obtain compound H1-102 (3.7 g, yield: 74%).

| Compound | MW | UV | PL | M.P. |
|---|---|---|---|---|
| H1-102 | 602.21 | 324 nm | 429 nm | 299° C. |

### Synthesis Example 10: Preparation of Compound H2-22

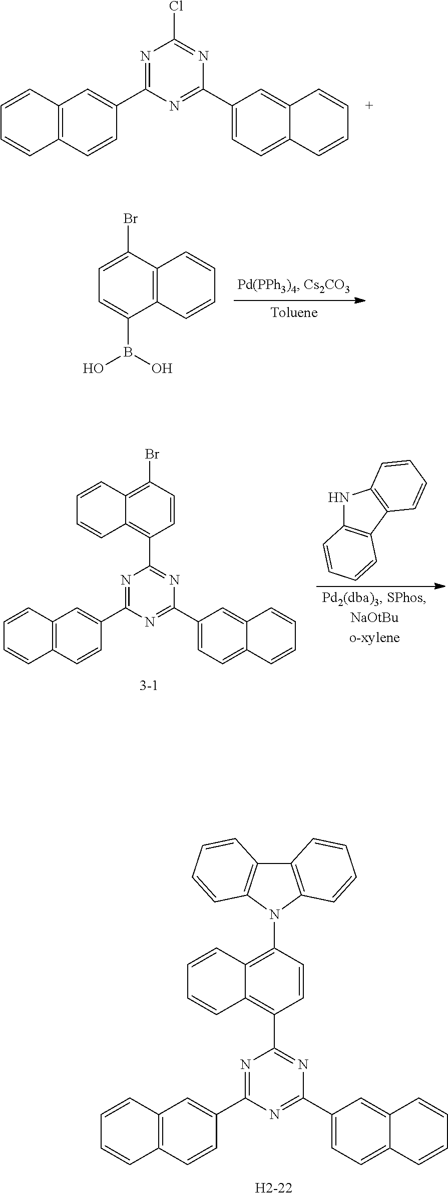

### Synthesis of Compound 3-1

2-chloro-4,6-di(naphthalen-2-yl)-1,3,5-triazine (20 g, 79.7 mmol), (4-bromonaphthalen-1-yl) boronic acid (32.2 g, 87.7 mmol), Pd(PPh₃)₄ (4.6 g, 3.985 mmol), and Cs₂CO₃ (65 g, 199.25 mmol) were dissolved in 400 mL of toluene in a flask, and the mixture was stirred under reflux for 4 hours. After completion of the reaction, the reaction mixture was cooled to room temperature, an organic layer was extracted with ethyl acetate, and the residual moisture was removed using magnesium sulfate. The residue was dried, and separated by column chromatography to obtain compound 3-1 (30 g, yield: 74%).

### Synthesis of Compound H2-22

Compound 3-1 (10 g, 19.7 mmol), 9H-carbazole (3.0 g, 17.9 mmol), tris(dibenzylideneacetone) dipalladium (0) (0.8 g, 0.9 mmol), dicyclohexylphosphino-2′,6′-dimethoxybiphenyl (s-phos) (0.73 g, 1.79 mmol), and sodium tert-butoxide (4.3 g, 44.75 mmol) were dissolved in 90 mL of xylene in a flask, and the mixture was stirred under reflux for 4 hours. After completion of the reaction, the reaction mixture was extracted with ethyl acetate, and separated by column chromatography to obtain compound H2-22 (1.5 g, yield: 13%).

| Compound | MW | M.P. |
|---|---|---|
| H2-22 | 624.75 | 265° C. |

### Synthesis Example 11: Preparation of Compound H2-115

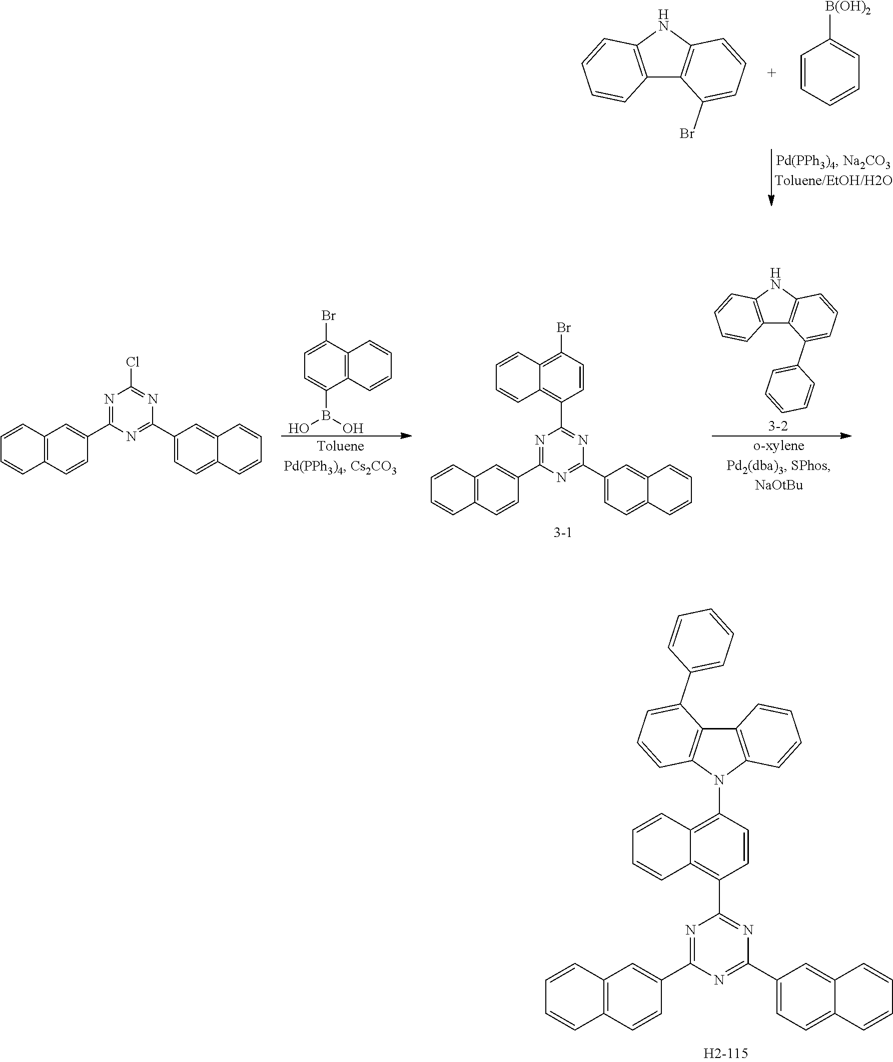

### Synthesis of Compound 3-2

4-bromo-9H-carbazole (10 g, 40.6 mmol), phenylboronic acid (6.2 g, 48.7 mmol), Pd(PPh₃)₄ (2.3 g, 2.03 mmol), and Na₂CO₃ (13 g, 121.8 mmol) were dissolved in 200 mL of toluene, 100 mL of ethanol, and 100 ml of water in a flask, and the mixture was stirred under reflux for 3 hours. After completion of the reaction, the reaction mixture was cooled to room temperature, an organic layer was extracted with ethyl acetate, and the residual moisture was removed using magnesium sulfate. The residue was dried, and separated by column chromatography to obtain compound 3-2 (9 g, yield: 91%).

### Synthesis of Compound H2-115

Compound 3-1 (8.5 g, 13.5 mmol), compound 3-2 (3.0 g, 12.3 mmol), Pd₂(dba)₃ (0.56 g, 0.615 mmol), s-phos (0.51 g, 1.23 mmol), and NaOtBu (2.9 g, 30.75 mmol) were dissolved in 60 mL of o-xylene in a flask, and the mixture was stirred under reflux for 4 hours. After completion of the reaction, the reaction mixture was extracted with ethyl acetate, and separated by column chromatography to obtain compound H2-115 (2.8 g, yield: 32.5%).

| Compound | MW | M.P. |
|---|---|---|
| H2-115 | 700.85 | 260.3° C. |

### Synthesis Example 12: Preparation of Compound H2-14

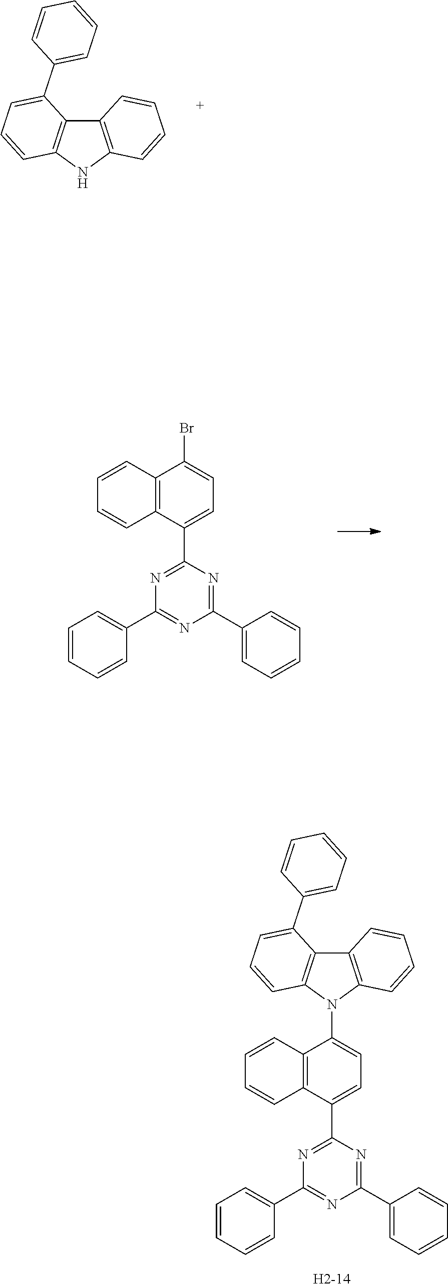 [SMILES: c1ccc(-c2cccc3[nH]c4ccccc4c23)cc1 ; Brc1ccc(-c2nc(-c3ccccc3)nc(-c3ccccc3)n2)c2ccccc12 ; c1ccc(-c2nc(-c3ccccc3)nc(-c3ccc(-n4c5ccccc5c5c(-c6ccccc6)cccc54)c4ccccc34)n2)cc1]

4-phenyl-9H-carbazole (3.0 g, 12.3 mmol), 2-(4-bromonaphthalen-1-yl)-4,6-diphenyl-1,3,5-triazine (5.4 g, 12.3 mmol), Pd₂(dba)₃ (0.56 g, 0.62 mmol), s-phos (0.51 g, 1.23 mmol), and NaOtBu (2.4 g, 24.7 mmol) were dissolved in 62 mL of o-xylene in a flask, and the mixture was stirred under reflux for 6 hours. After completion of the reaction, the reaction mixture was cooled to room temperature, and MeOH was added thereto, while stirring it at room temperature, to produce a solid. The solid was filtered under reduced pressure, extracted with MC/Hex, and separated by column chromatography to obtain compound H2-14 (3.3 g, yield: 45%).

| Compound | MW | M.P. |
|---|---|---|
| H2-14 | 600.71 | 254° C. |

### Synthesis Example 13: Preparation of Compound H2-16

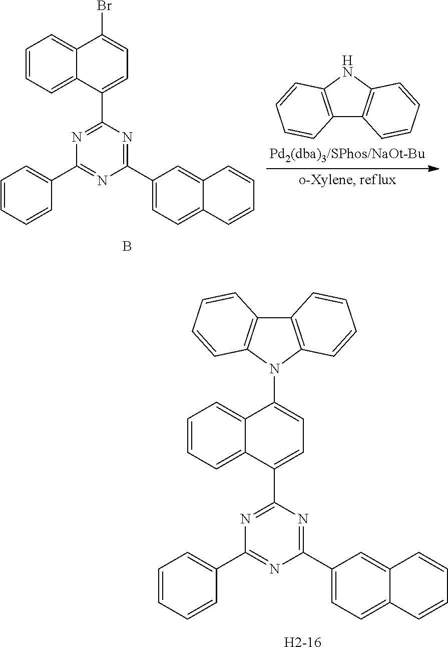

Compound B (8.0 g, 16.4 mmol), 9H-carbazole (3.0 g, 18.0 mmol), Pd₂(dba)₃ (0.8 g, 0.8 mmol), s-phos (0.7 g, 1.64 mmol), and NaOtBu (2.4 g, 24.6 mmol) were dissolved in 82 mL of o-xylene in a flask, and the mixture was stirred under reflux for 4 hours. After completion of the reaction, the reaction mixture was extracted with ethyl acetate, and separated by column chromatography to obtain compound H2-16 (6.0 g, yield: 69%).

| Compound | MW | M.P. |
|---|---|---|
| H2-16 | 524.63 | 245° C. |

### Synthesis Example 14: Preparation of Compound H2-116

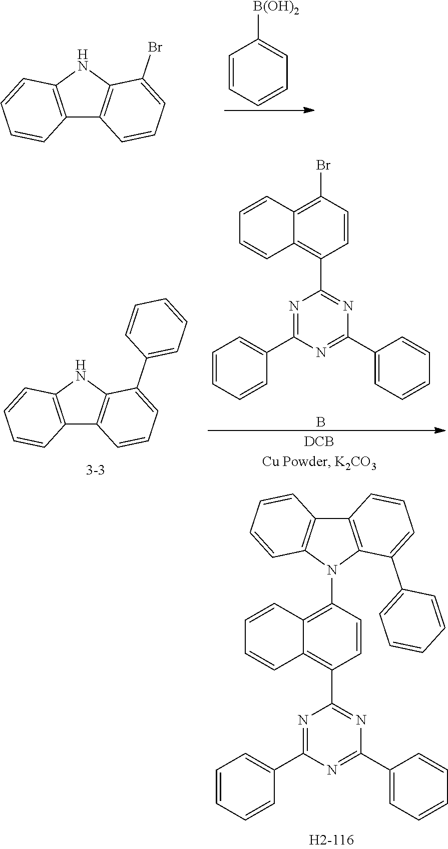

### Synthesis of Compound 3-3

1-bromo-9H-carbazole (10 g, 40.6 mmol), phenylboronic acid (6.2 g, 48.7 mmol), Pd(PPh₃)₄ (2.3 g, 2.03 mmol), and Na₂CO₃ (13 g, 121.8 mmol) were dissolved in 200 ml of toluene, 100 mL of ethanol, and 100 ml of water in a flask, and the mixture was stirred under reflux for 3 hours. After completion of the reaction, the reaction mixture was cooled to room temperature, an organic layer was extracted with ethyl acetate, and the residual moisture was removed using magnesium sulfate. The residue was dried, and separated by column chromatography to obtain compound 3-3 (9 g, yield: 96%).

### Synthesis of Compound H2-116

Compound 3-3 (3.0 g, 12.3 mmol), compound B (8 g, 18.5 mmol), Cu powder (0.39 g, 6.15 mmol), and K₂CO₃ (3.4 g, 24.6 mmol) were dissolved in 60 mL of dichlorobenzene (DCB) in a flask, and the mixture was stirred under reflux for 24 hours. After completion of the reaction, the reaction mixture was cooled to room temperature, and MeOH was added thereto, while stirring it at room temperature, to produce a solid. The solid was filtered under reduced pressure, extracted with MC/Hex, and separated by column chromatography to obtain compound H2-116 (1.1 g, yield: 14.8%).

| Compound | MW | M.P. |
|---|---|---|
| H2-116 | 600.23 | 226.9° C. |

### Device Examples 1 and 2: Producing an OLED Deposited with the Plurality of Host Materials According to the Present Disclosure as a Host

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 shown in Table 2 below was introduced into a cell of the vacuum vapor deposition apparatus, and compound HT-1 shown in Table 2 below was introduced into another cell of the vacuum vapor deposition apparatus. The two materials were evaporated at different rates, and compound HI-1 was deposited in a doping amount of 3 wt % based on the total amount of compound HI-1 and compound HT-1 to form a first hole injection layer having a thickness of 10 nm on the ITO substrate. Next, compound HT-1 was deposited on the first hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The first host compound and the second host compound shown in Table 1 below were introduced into two cells of the vacuum vapor depositing apparatus as hosts, and compound D-39 was introduced into another cell as a dopant. The two host materials were evaporated at a rate of 1:1 and the dopant material was simultaneously evaporated at a different rate, and the dopant was deposited in a doping amount of 3 wt % based on the total amount of the hosts and the dopant to form a light-emitting layer having a thickness of 40 nm on the second hole transport layer. Compound ET-1 and compound EI-1 were evaporated in a weight ratio of 50:50 to form an electron transport layer having a thickness of 35 nm on the light-emitting layer. After depositing compound EI-1 as an electron injection layer having a thickness of 2 nm on the electron transport layer, an AI cathode having a thickness of 80 nm was deposited on the electron injection layer by another vacuum vapor deposition apparatus. Thus, an OLED was produced. All the materials used for producing the OLED were purified by vacuum sublimation at 10-6 torr.

### Comparative Example: Producing an OLED Comprising the Comparative Compound as a Host

An OLED was produced in the same manner as in Device Examples 1 and 2, except that the second host compound shown in Table 1 below was used alone as a host of the light-emitting layer.

The driving voltage, luminous efficiency, and light-emitting color at a luminance of 1,000 nit, and the time taken for luminance to decrease from 100% to 95% at a luminance of 5,500 nit (lifetime; T95) of the OLEDs produced in Device Examples 1 and 2, and the Comparative Example are provided in Table 1 below.

TABLE 1

| | | | Driving | Luminous | Light- | |
|---|---|---|---|---|---|---|
| | First | Second | Voltage | Efficiency | Emitting | Lifetime |
| | Host | Host | [V] | [cd/A] | Color | T95[hr] |
| Device | H1-80 | H2-115 | 3.5 | 33.4 | Red | 310 |
| Example 1 | | | | | | |
| Device | H1-80 | H2-16 | 3.2 | 35.3 | Red | 367 |
| Example 2 | | | | | | |
| Comparative | — | H2-115 | 4.0 | 25.6 | Red | 45.9 |
| Example | | | | | | |

From Table 1 above, it can be seen that the OLEDs comprising the specific combination of compounds according to the present disclosure as host materials can significantly lower the driving voltage and have remarkably improved luminous efficiency and lifetime properties compared to the conventional OLED using a single host material (Comparative Example).

TABLE 2

| | |
|---|---|
| Hole Injection Layer/ Hole Transport Layer | 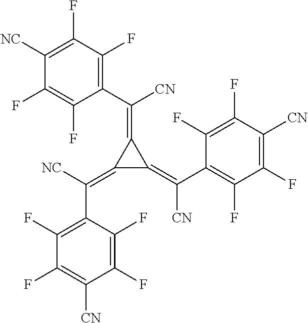 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 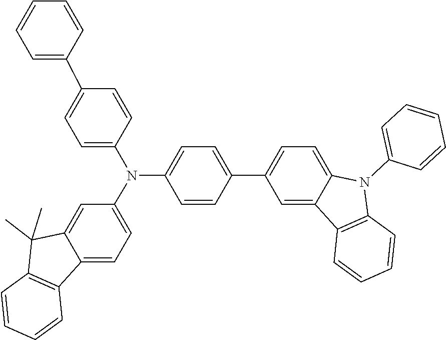 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | HT-1 |
| | 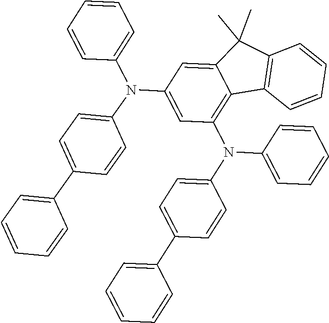 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HT-2 |
| Light-Emitting Layer | 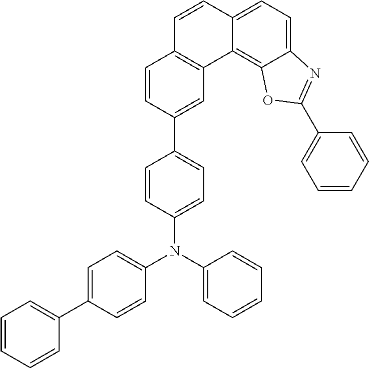 [SMILES: c1ccc(-c2ccc(N(c3ccccc3)c3ccc(-c4ccc5ccc6ccc7nc(-c8ccccc8)oc7c6c5c4)cc3)cc2)cc1] |
| | H1-80 |
| | 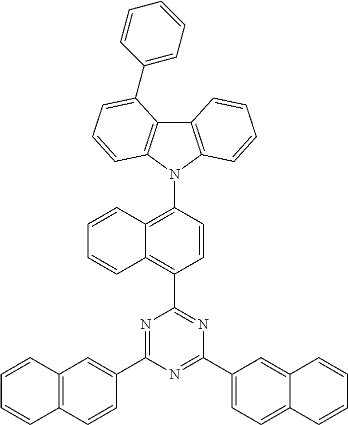 [SMILES: c1ccc(-c2cccc3c2c2ccccc2n3-c2ccc(-c3nc(-c4ccc5ccccc5c4)nc(-c4ccc5ccccc5c4)n3)c3ccccc23)cc1] |
| | H2-16 |
| | 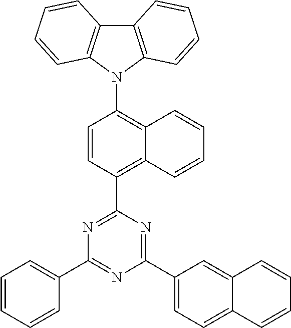 [SMILES: c1ccc(-c2nc(-c3ccc4ccccc4c3)nc(-c3ccc(-n4c5ccccc5c5ccccc54)c4ccccc34)n2)cc1] |
| | H2-115 |
| | 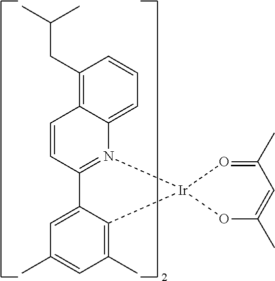 |
| | D-39 |
| Electron Transport Layer/ Electron Injection Layer | 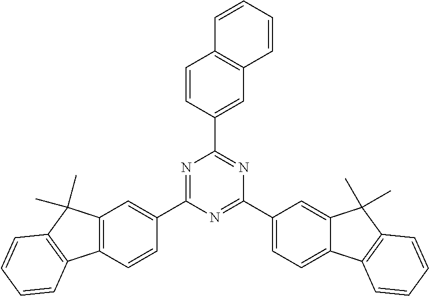 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ET-1 |
| | 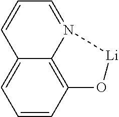 |
| | EI-1 |

1. A plurality of host materials comprising a first host material comprising a compound represented by the following formula 1-1 or 1-2, and a second host material comprising a compound represented by the following formula 2: 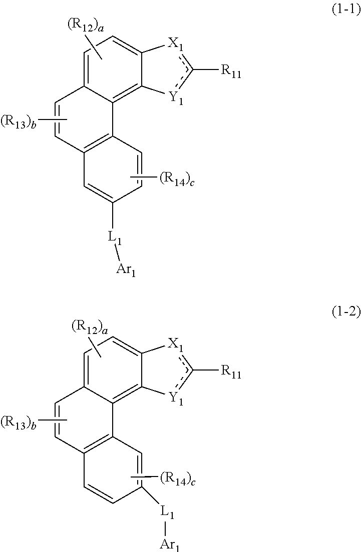
wherein
X₁ represents —N═;
Y₁ represents —O—, or —S;
L₁ represents a single bond, or a deuterium-substituted or unsubstituted (C6-C30) arylene;
Ar₁ represents a di-(C6-C30) arylamino unsubstituted or substituted with at least one of deuterium and (C1-C30)alkyl, or a (C6-C30) aryl(3- to 30-membered) heteroarylamino unsubstituted or substituted with at least one of deuterium and (C6-C30) aryl;
R₁₁ represents a deuterium-substituted or unsubstituted (C6-C30) aryl;
R₁₂ to R₁₄ each independently represent hydrogen or deuterium;
a and b each independently represent 1 or 2, and c is an integer of 1 to 3, where if a to c are an integer of 2 or more, each of R₁₂ to each of R₁₄ may be the same or different; 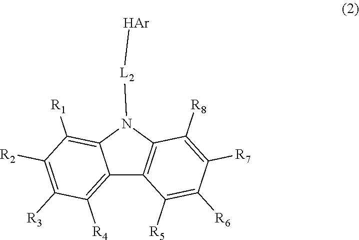
wherein
HAr represents a substituted triazinyl having two substituents, wherein one substituent is a naphthyl unsubstituted or substituted with deuterium, and the other substituent is selected from the group consisting of phenyl, naphthyl, biphenyl, phenanthrenyl and terphenyl, and each optionally further substituted with deuterium;
L₂ represents a naphthylene unsubstituted or substituted with deuterium; and
R₁ to R₈ each independently represent hydrogen, deuterium, or a (C6-C30) aryl unsubstituted or substituted with deuterium.

2. The plurality of host materials according to claim 1, wherein the compound represented by formula 1-1 or 1-2 is at least one selected from the following compounds:

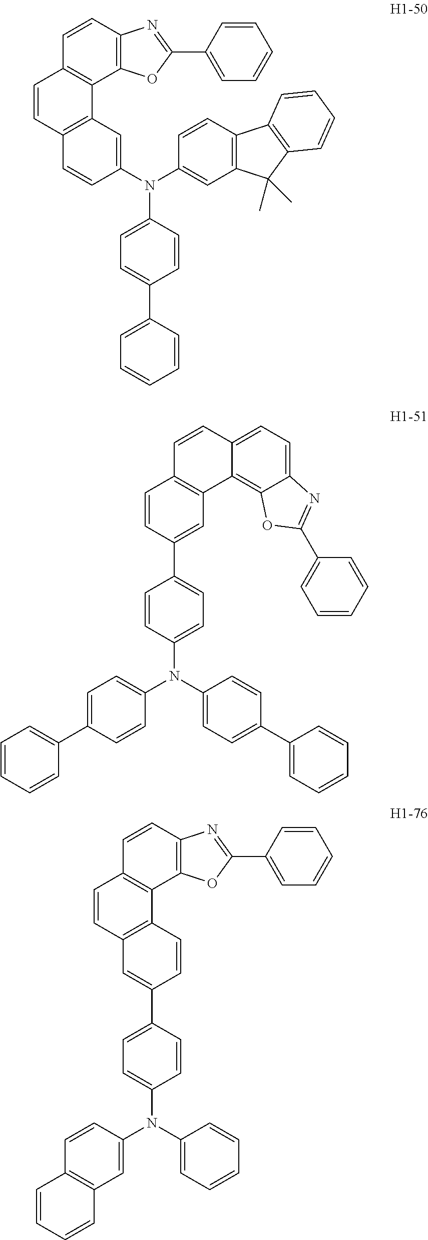

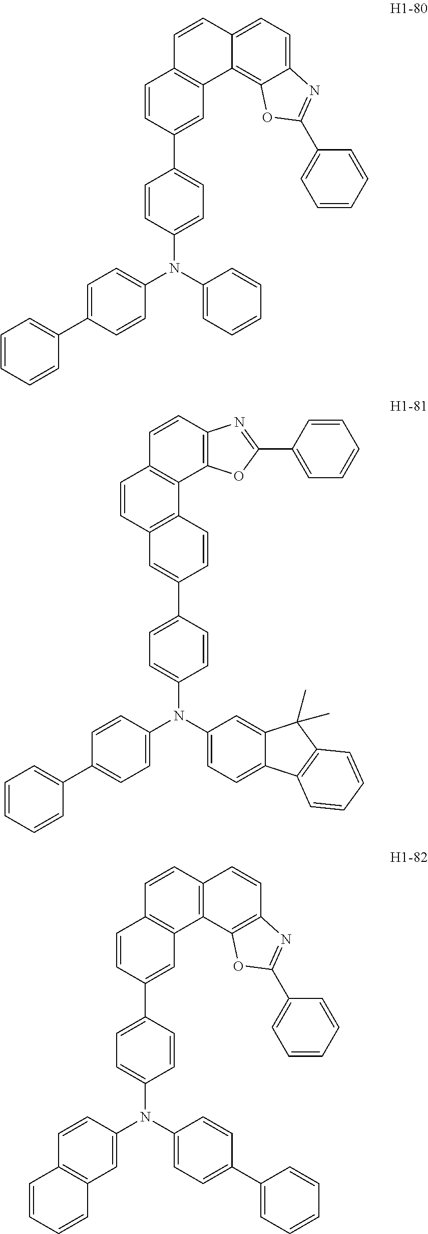 [SMILES: c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1]

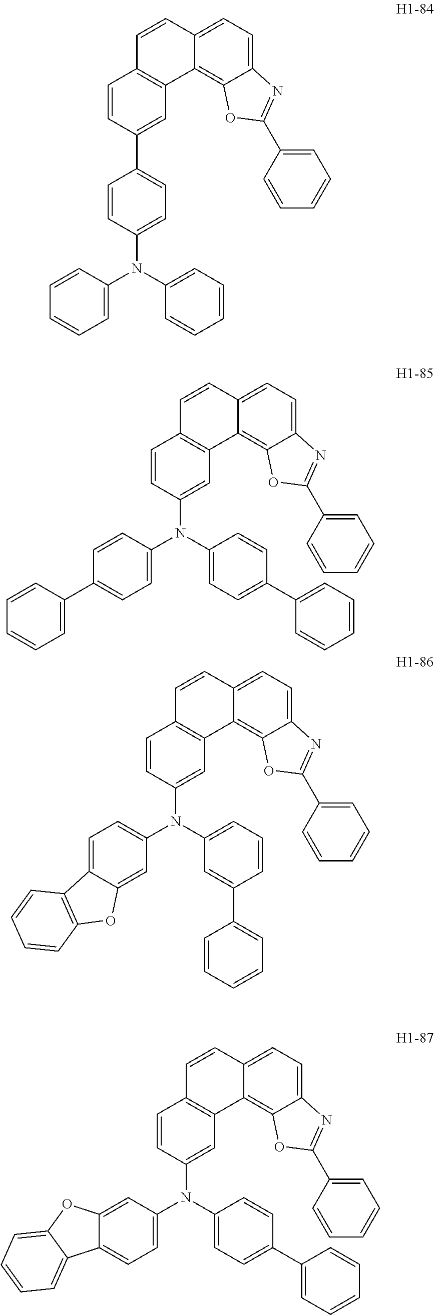 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc4c(c3)oc3ccccc34)c3ccc4ccc5ccc6nc(-c7ccccc7)oc6c5c4c3)cc2)cc1]

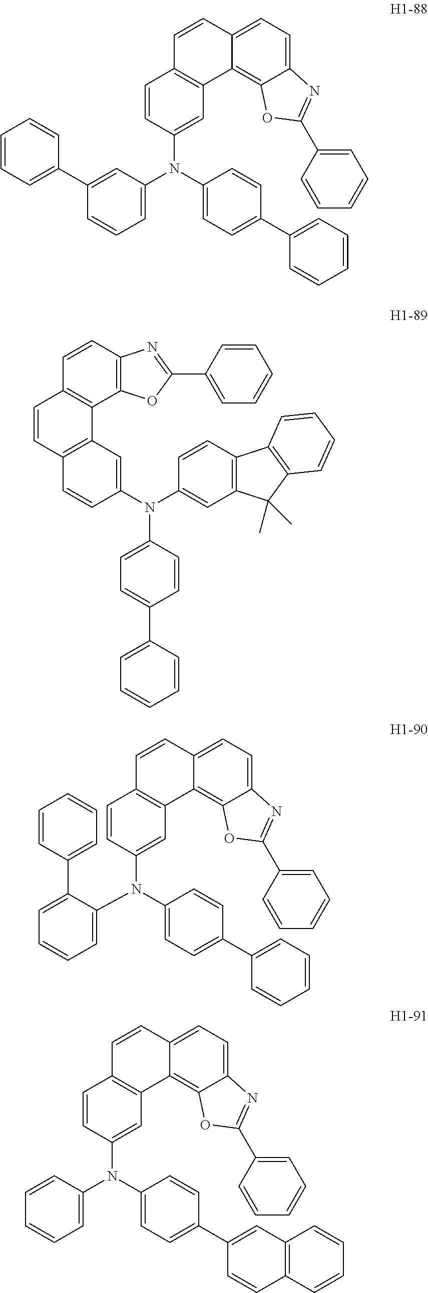

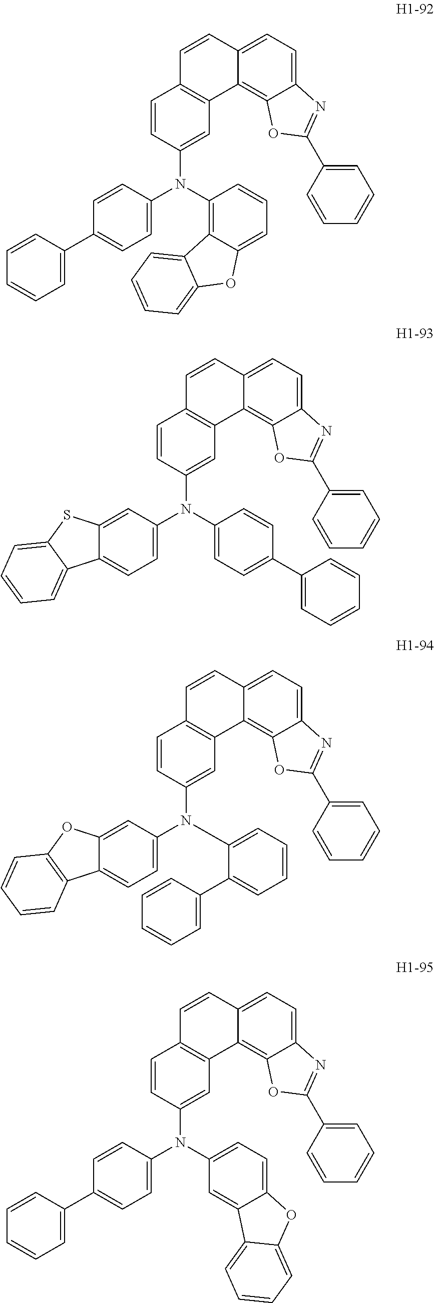 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1]

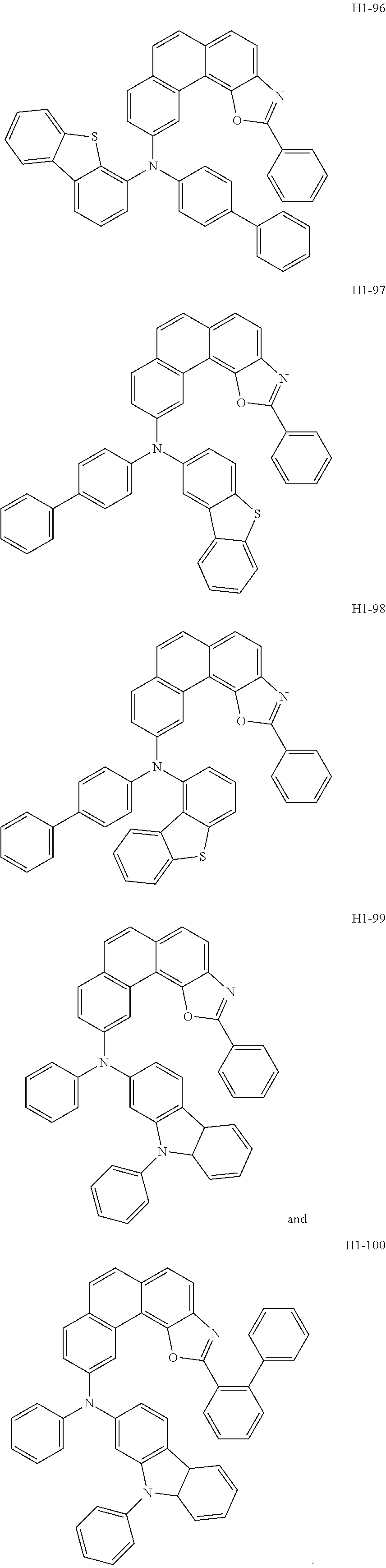

3. The plurality of host materials according to claim 1, wherein the compound represented by formula 2 is at least one selected from the following compounds:

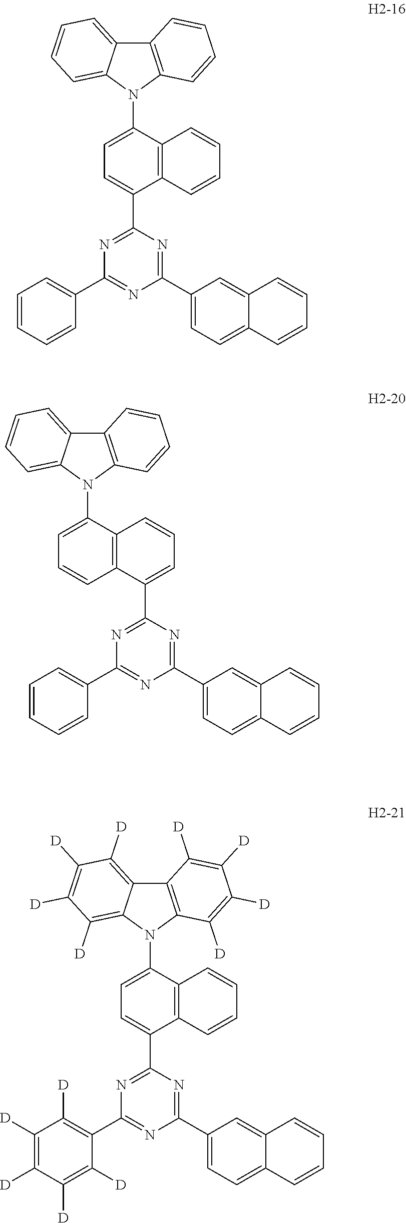 [SMILES: c1ccc(-c2nc(-c3ccc4ccccc4c3)nc(-c3ccc(-n4c5ccccc5c5ccccc54)c4ccccc34)n2)cc1 ; c1ccc(-c2nc(-c3ccc4ccccc4c3)nc(-c3cccc4c(-n5c6ccccc6c6ccccc65)cccc34)n2)cc1 ; [2H]c1c([2H])c([2H])c(-c2nc(-c3ccc4ccccc4c3)nc(-c3ccc(-n4c5c([2H])c([2H])c([2H])c([2H])c5c5c([2H])c([2H])c([2H])c([2H])c54)c4ccccc34)n2)c([2H])c1[2H]]

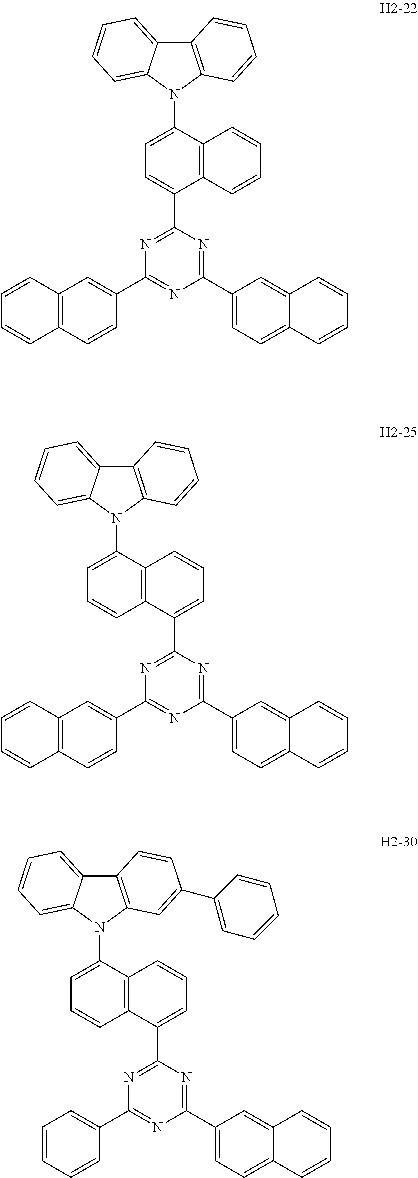 [SMILES: c1ccc2cc(-c3nc(-c4ccc5ccccc5c4)nc(-c4ccc(-n5c6ccccc6c6ccccc65)c5ccccc45)n3)ccc2c1 ; c1ccc2cc(-c3nc(-c4ccc5ccccc5c4)nc(-c4cccc5c(-n6c7ccccc7c7ccccc76)cccc45)n3)ccc2c1 ; c1ccc(-c2ccc3c4ccccc4n(-c4cccc5c(-c6nc(-c7ccccc7)nc(-c7ccc8ccccc8c7)n6)cccc45)c3c2)cc1]

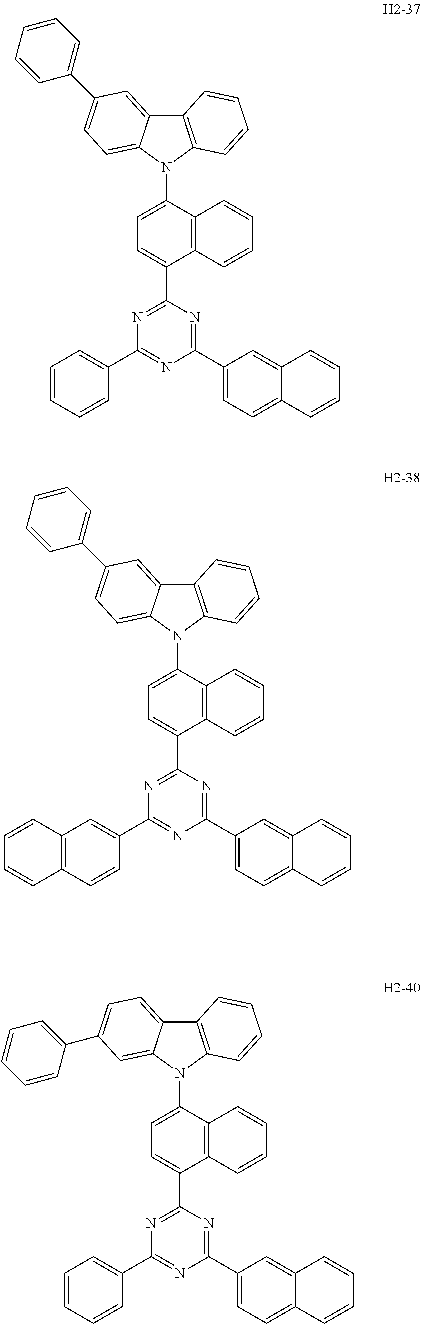 [SMILES: c1ccc(-c2ccc3c(c2)c2ccccc2n3-c2ccc(-c3nc(-c4ccccc4)nc(-c4ccc5ccccc5c4)n3)c3ccccc23)cc1 ; c1ccc(-c2ccc3c(c2)c2ccccc2n3-c2ccc(-c3nc(-c4ccc5ccccc5c4)nc(-c4ccc5ccccc5c4)n3)c3ccccc23)cc1 ; c1ccc(-c2ccc3c4ccccc4n(-c4ccc(-c5nc(-c6ccccc6)nc(-c6ccc7ccccc7c6)n5)c5ccccc45)c3c2)cc1]

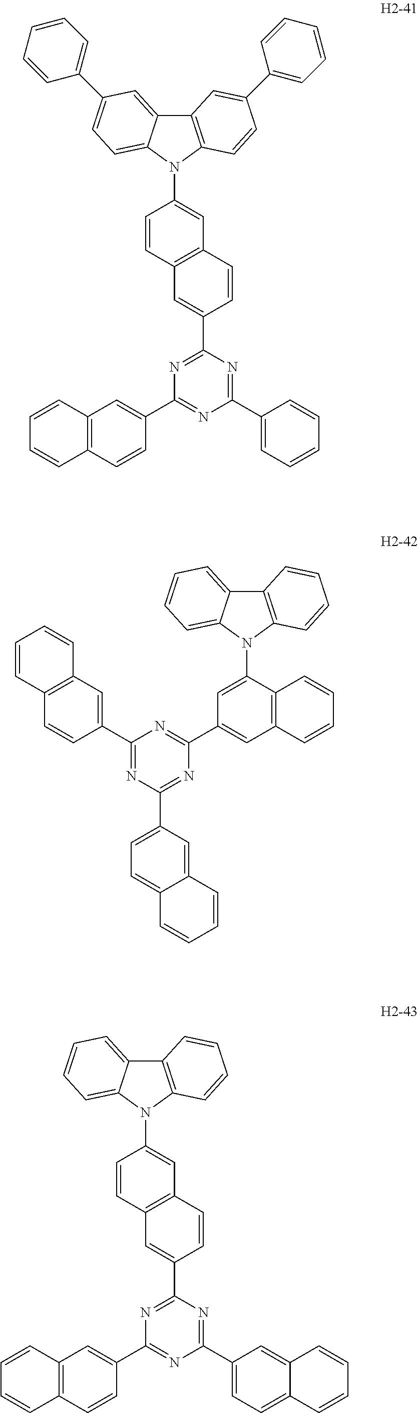 [SMILES: c1ccc(-c2ccc3c(c2)c2cc(-c4ccccc4)ccc2n3-c2ccc3cc(-c4nc(-c5ccccc5)nc(-c5ccc6ccccc6c5)n4)ccc3c2)cc1 ; c1ccc2cc(-c3nc(-c4ccc5ccccc5c4)nc(-c4cc(-n5c6ccccc6c6ccccc65)c5ccccc5c4)n3)ccc2c1 ; c1ccc2cc(-c3nc(-c4ccc5ccccc5c4)nc(-c4ccc5cc(-n6c7ccccc7c7ccccc76)ccc5c4)n3)ccc2c1]

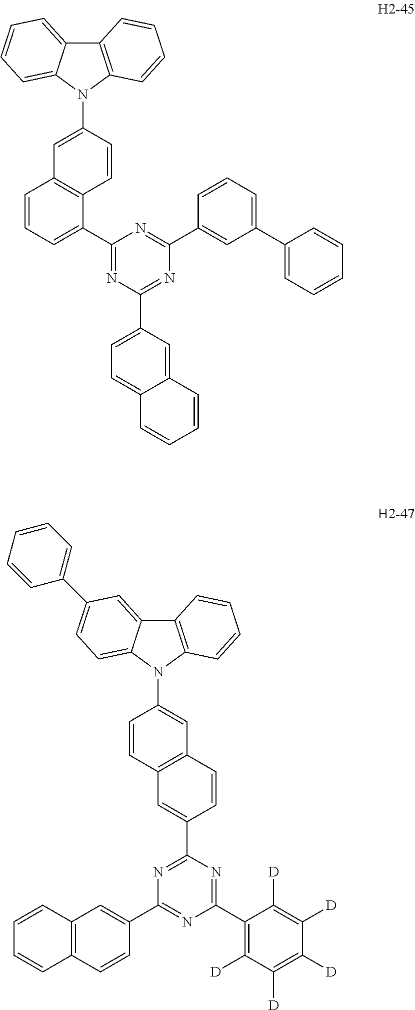 [SMILES: c1ccc(-c2cccc(-c3nc(-c4ccc5ccccc5c4)nc(-c4cccc5cc(-n6c7ccccc7c7ccccc76)ccc45)n3)c2)cc1 ; [2H]c1c([2H])c([2H])c(-c2nc(-c3ccc4ccccc4c3)nc(-c3ccc4cc(-n5c6ccccc6c6cc(-c7ccccc7)ccc65)ccc4c3)n2)c([2H])c1[2H]]

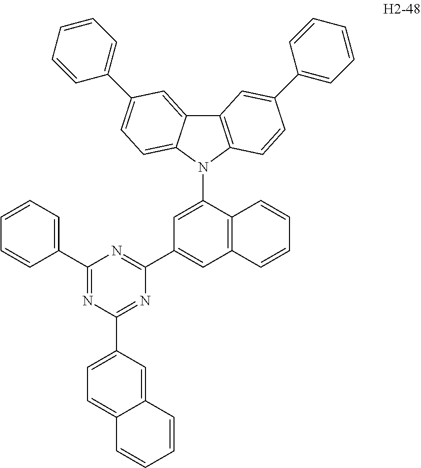 [SMILES: c1ccc(-c2ccc3c(c2)c2cc(-c4ccccc4)ccc2n3-c2cc(-c3nc(-c4ccccc4)nc(-c4ccc5ccccc5c4)n3)cc3ccccc23)cc1]

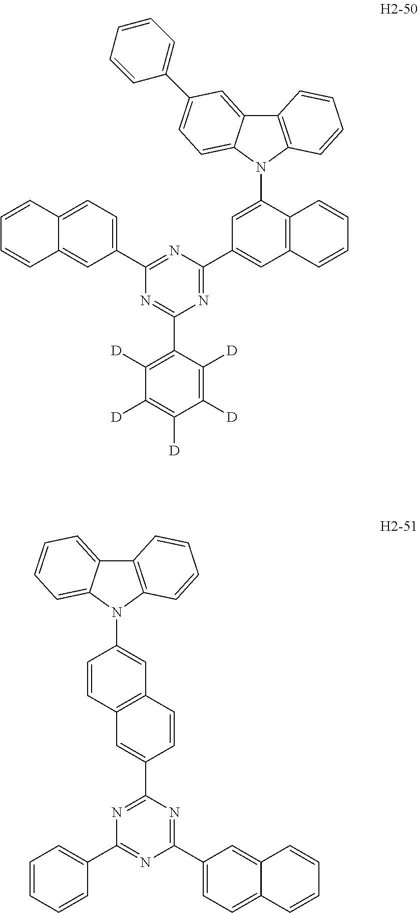 [SMILES: [2H]c1c([2H])c([2H])c(-c2nc(-c3ccc4ccccc4c3)nc(-c3cc(-n4c5ccccc5c5cc(-c6ccccc6)ccc54)c4ccccc4c3)n2)c([2H])c1[2H] ; c1ccc(-c2nc(-c3ccc4ccccc4c3)nc(-c3ccc4cc(-n5c6ccccc6c6ccccc65)ccc4c3)n2)cc1]

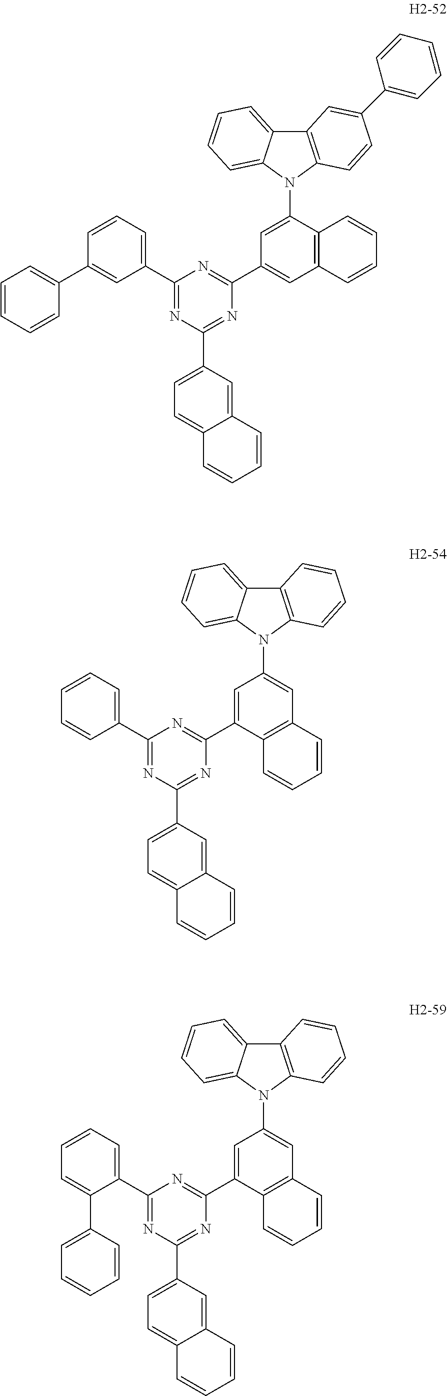 [SMILES: c1ccc(-c2cccc(-c3nc(-c4ccc5ccccc5c4)nc(-c4cc(-n5c6ccccc6c6cc(-c7ccccc7)ccc65)c5ccccc5c4)n3)c2)cc1 ; c1ccc(-c2nc(-c3ccc4ccccc4c3)nc(-c3cc(-n4c5ccccc5c5ccccc54)cc4ccccc34)n2)cc1 ; c1ccc(-c2ccccc2-c2nc(-c3ccc4ccccc4c3)nc(-c3cc(-n4c5ccccc5c5ccccc54)cc4ccccc34)n2)cc1]

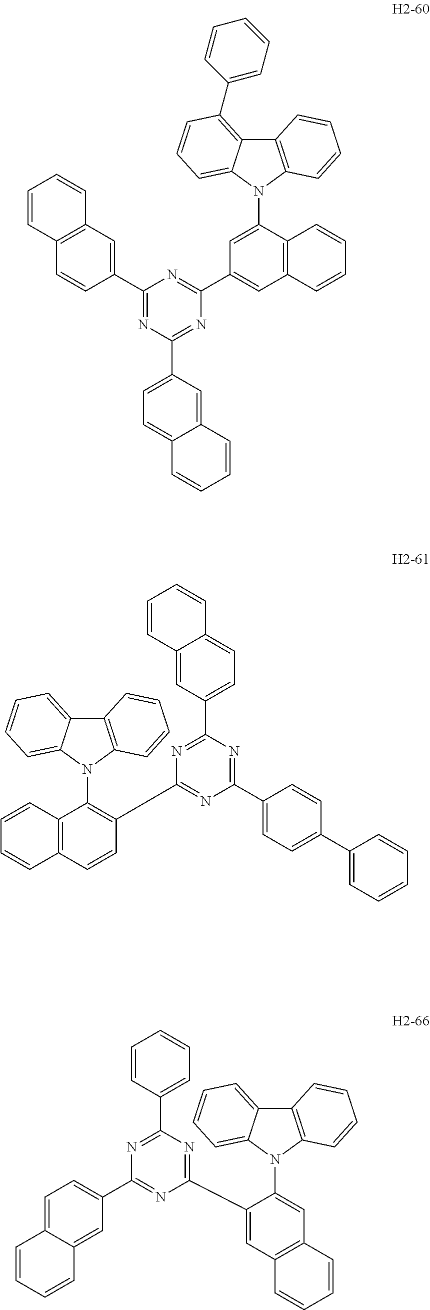 [SMILES: c1ccc(-c2cccc3c2c2ccccc2n3-c2cc(-c3nc(-c4ccc5ccccc5c4)nc(-c4ccc5ccccc5c4)n3)cc3ccccc23)cc1 ; c1ccc(-c2ccc(-c3nc(-c4ccc5ccccc5c4)nc(-c4ccc5ccccc5c4-n4c5ccccc5c5ccccc54)n3)cc2)cc1 ; c1ccc(-c2nc(-c3ccc4ccccc4c3)nc(-c3cc4ccccc4cc3-n3c4ccccc4c4ccccc43)n2)cc1]

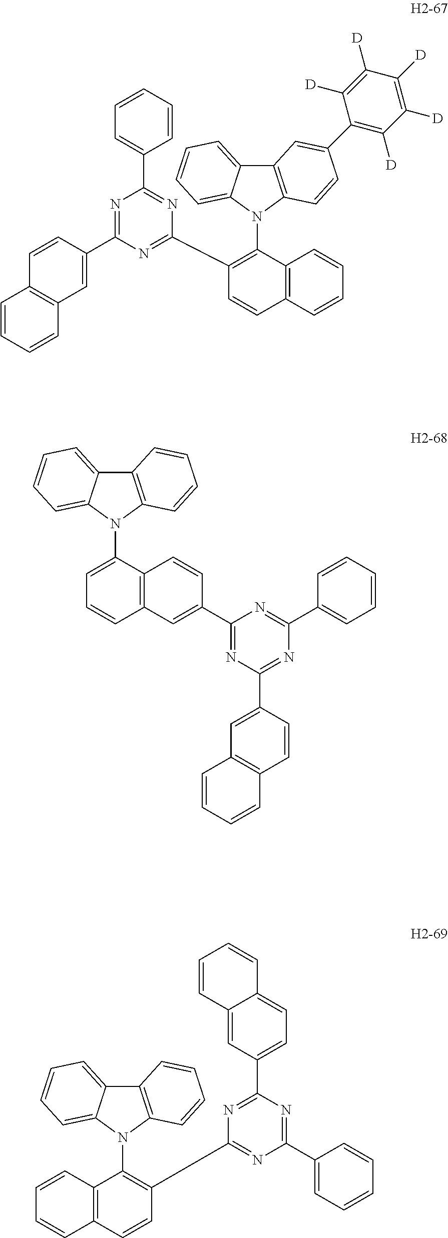 [SMILES: [2H]c1c([2H])c([2H])c(-c2ccc3c(c2)c2ccccc2n3-c2c(-c3nc(-c4ccccc4)nc(-c4ccc5ccccc5c4)n3)ccc3ccccc23)c([2H])c1[2H] ; c1ccc(-c2nc(-c3ccc4ccccc4c3)nc(-c3ccc4c(-n5c6ccccc6c6ccccc65)cccc4c3)n2)cc1 ; c1ccc(-c2nc(-c3ccc4ccccc4c3)nc(-c3ccc4ccccc4c3-n3c4ccccc4c4ccccc43)n2)cc1]

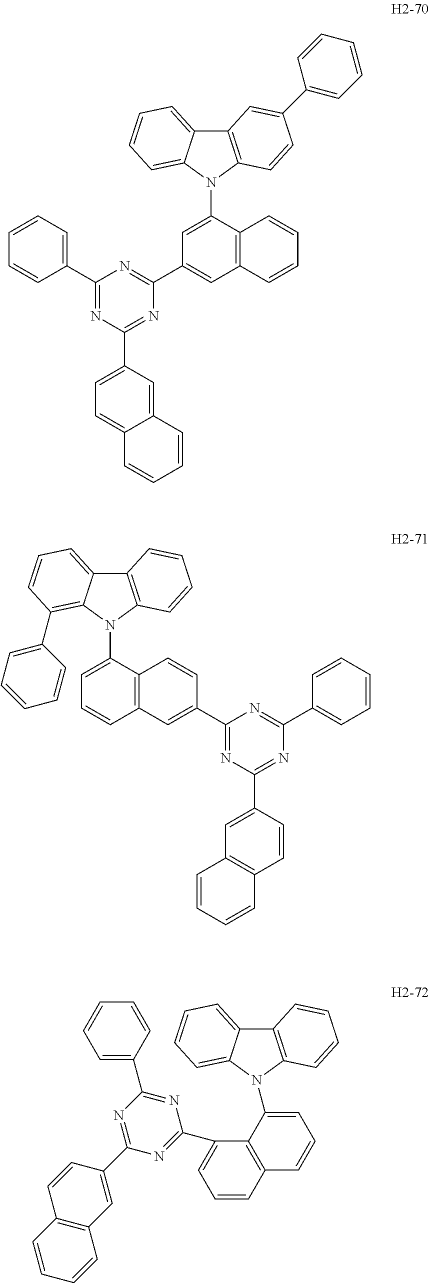 [SMILES: c1ccc(-c2ccc3c(c2)c2ccccc2n3-c2cc(-c3nc(-c4ccccc4)nc(-c4ccc5ccccc5c4)n3)cc3ccccc23)cc1 ; c1ccc(-c2nc(-c3ccc4ccccc4c3)nc(-c3ccc4c(-n5c6ccccc6c6cccc(-c7ccccc7)c65)cccc4c3)n2)cc1 ; c1ccc(-c2nc(-c3ccc4ccccc4c3)nc(-c3cccc4cccc(-n5c6ccccc6c6ccccc65)c34)n2)cc1]

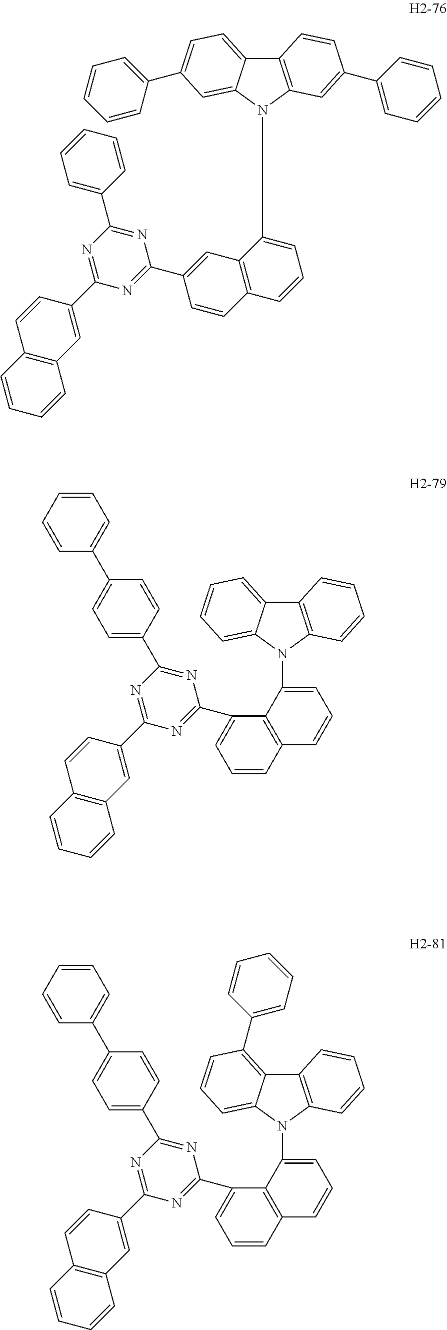 [SMILES: c1ccc(-c2ccc3c4ccc(-c5ccccc5)cc4n(-c4cccc5ccc(-c6nc(-c7ccccc7)nc(-c7ccc8ccccc8c7)n6)cc45)c3c2)cc1 ; c1ccc(-c2ccc(-c3nc(-c4ccc5ccccc5c4)nc(-c4cccc5cccc(-n6c7ccccc7c7ccccc76)c45)n3)cc2)cc1 ; c1ccc(-c2ccc(-c3nc(-c4ccc5ccccc5c4)nc(-c4cccc5cccc(-n6c7ccccc7c7c(-c8ccccc8)cccc76)c45)n3)cc2)cc1]

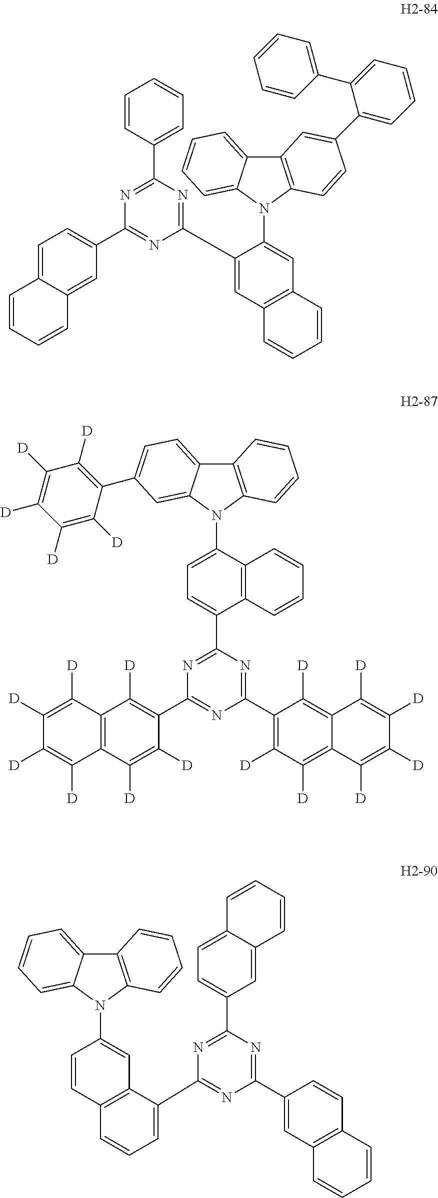 [SMILES: c1ccc(-c2nc(-c3ccc4ccccc4c3)nc(-c3cc4ccccc4cc3-n3c4ccccc4c4cc(-c5ccccc5-c5ccccc5)ccc43)n2)cc1 ; [2H]c1c([2H])c([2H])c(-c2ccc3c4ccccc4n(-c4ccc(-c5nc(-c6c([2H])c([2H])c7c([2H])c([2H])c([2H])c([2H])c7c6[2H])nc(-c6c([2H])c([2H])c7c([2H])c([2H])c([2H])c([2H])c7c6[2H])n5)c5ccccc45)c3c2)c([2H])c1[2H] ; c1ccc2cc(-c3nc(-c4ccc5ccccc5c4)nc(-c4cccc5ccc(-n6c7ccccc7c7ccccc76)cc45)n3)ccc2c1]

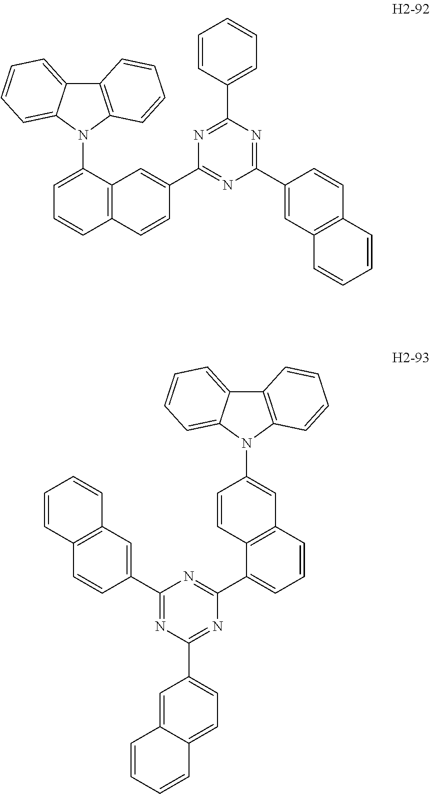 [SMILES: c1ccc(-c2nc(-c3ccc4ccccc4c3)nc(-c3ccc4cccc(-n5c6ccccc6c6ccccc65)c4c3)n2)cc1 ; c1ccc2cc(-c3nc(-c4ccc5ccccc5c4)nc(-c4cccc5cc(-n6c7ccccc7c7ccccc76)ccc45)n3)ccc2c1]

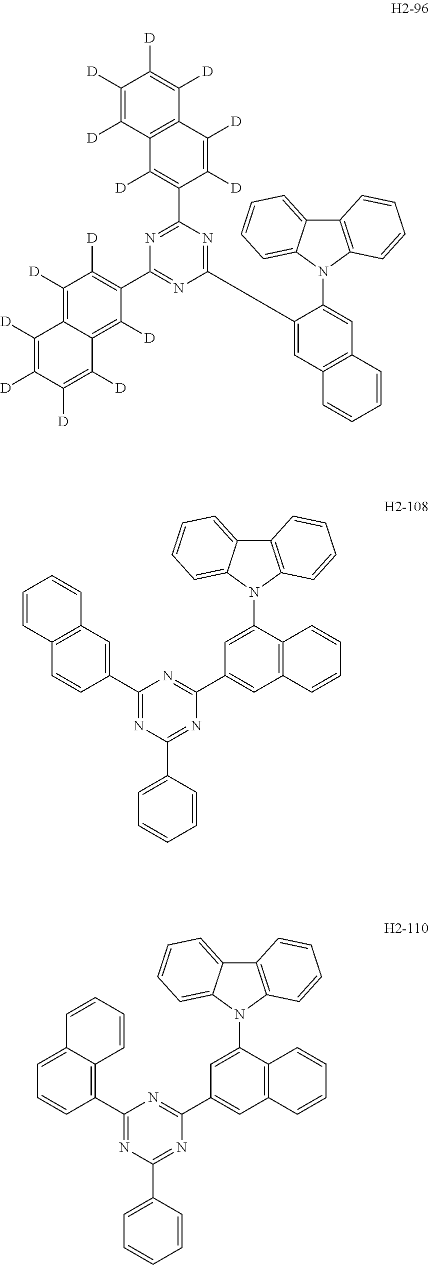 [SMILES: [2H]c1c([2H])c([2H])c2c([2H])c(-c3nc(-c4cc5ccccc5cc4-n4c5ccccc5c5ccccc54)nc(-c4c([2H])c([2H])c5c([2H])c([2H])c([2H])c([2H])c5c4[2H])n3)c([2H])c([2H])c2c1[2H] ; c1ccc(-c2nc(-c3ccc4ccccc4c3)nc(-c3cc(-n4c5ccccc5c5ccccc54)c4ccccc4c3)n2)cc1 ; c1ccc(-c2nc(-c3cc(-n4c5ccccc5c5ccccc54)c4ccccc4c3)nc(-c3cccc4ccccc34)n2)cc1]

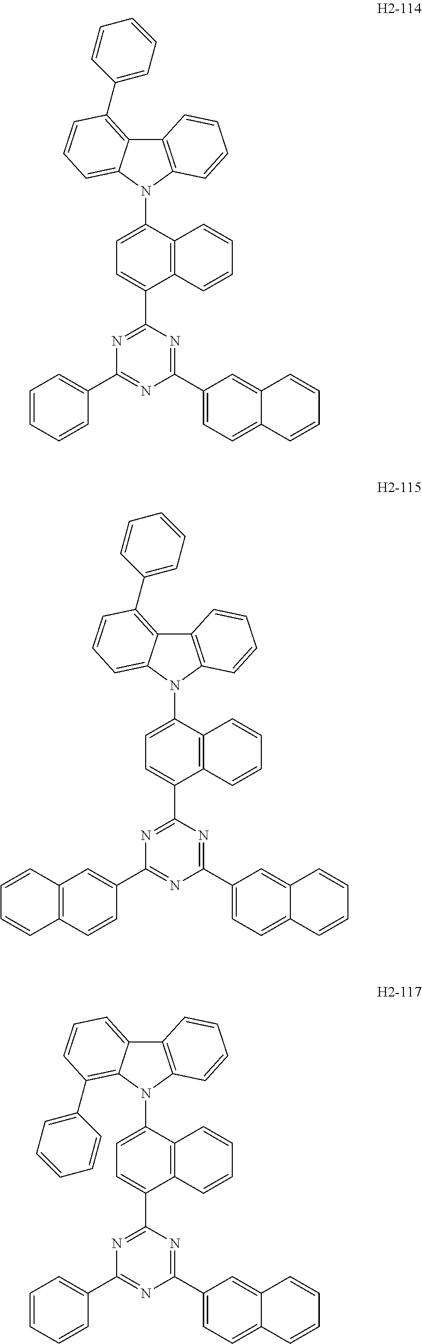 [SMILES: c1ccc(-c2nc(-c3ccc4ccccc4c3)nc(-c3ccc(-n4c5ccccc5c5c(-c6ccccc6)cccc54)c4ccccc34)n2)cc1 ; c1ccc(-c2cccc3c2c2ccccc2n3-c2ccc(-c3nc(-c4ccc5ccccc5c4)nc(-c4ccc5ccccc5c4)n3)c3ccccc23)cc1 ; c1ccc(-c2nc(-c3ccc4ccccc4c3)nc(-c3ccc(-n4c5ccccc5c5cccc(-c6ccccc6)c54)c4ccccc34)n2)cc1]

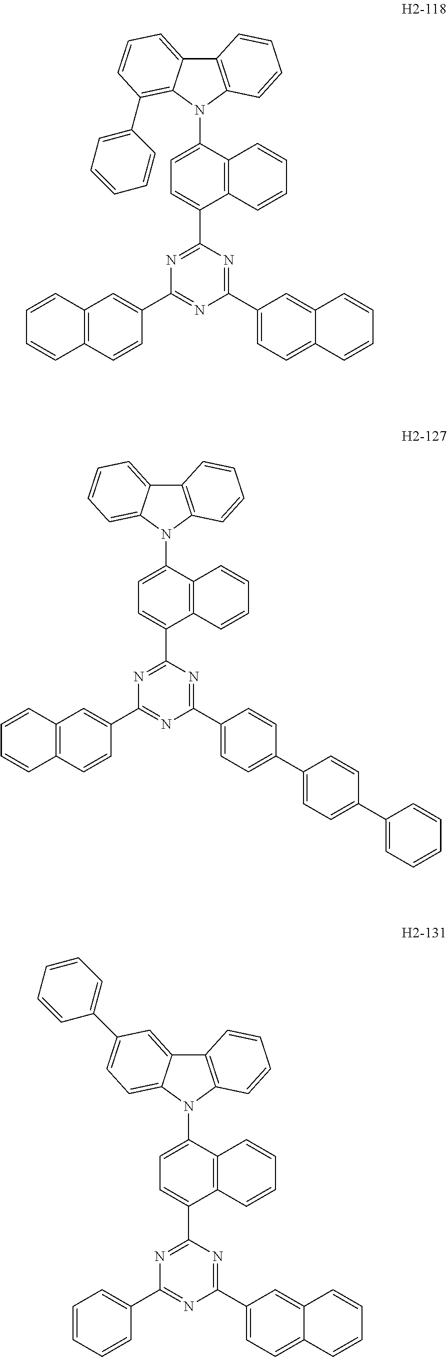 [SMILES: c1ccc(-c2cccc3c4ccccc4n(-c4ccc(-c5nc(-c6ccc7ccccc7c6)nc(-c6ccc7ccccc7c6)n5)c5ccccc45)c23)cc1 ; c1ccc(-c2ccc(-c3ccc(-c4nc(-c5ccc6ccccc6c5)nc(-c5ccc(-n6c7ccccc7c7ccccc76)c6ccccc56)n4)cc3)cc2)cc1 ; c1ccc(-c2ccc3c(c2)c2ccccc2n3-c2ccc(-c3nc(-c4ccccc4)nc(-c4ccc5ccccc5c4)n3)c3ccccc23)cc1]

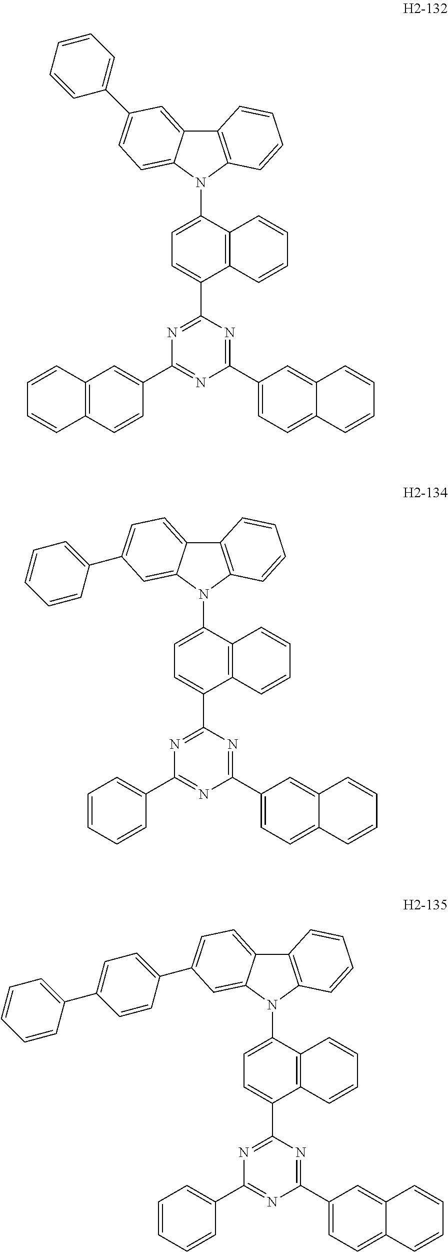 [SMILES: c1ccc(-c2ccc3c(c2)c2ccccc2n3-c2ccc(-c3nc(-c4ccc5ccccc5c4)nc(-c4ccc5ccccc5c4)n3)c3ccccc23)cc1 ; c1ccc(-c2ccc3c4ccccc4n(-c4ccc(-c5nc(-c6ccccc6)nc(-c6ccc7ccccc7c6)n5)c5ccccc45)c3c2)cc1 ; c1ccc(-c2ccc(-c3ccc4c5ccccc5n(-c5ccc(-c6nc(-c7ccccc7)nc(-c7ccc8ccccc8c7)n6)c6ccccc56)c4c3)cc2)cc1]

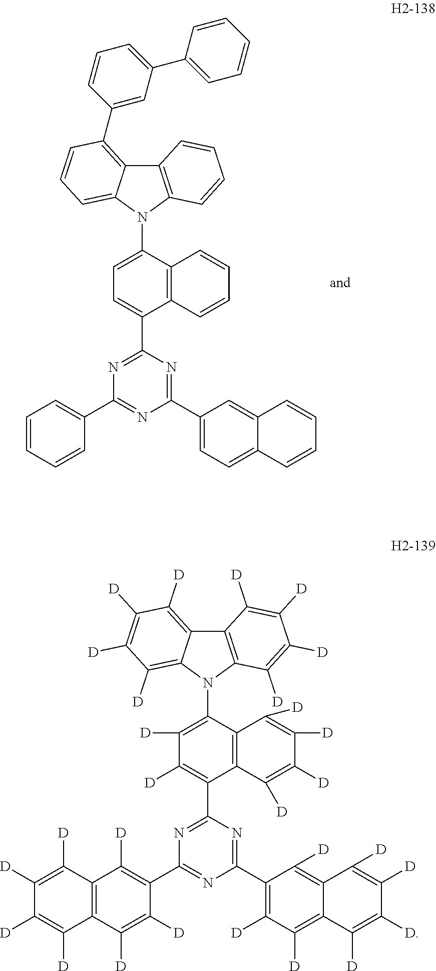

4. An organic electroluminescent device comprising an anode, a cathode, and at least one light-emitting layer between the anode and the cathode, wherein at least one of the light-emitting layers comprises the plurality of host materials according to claim 1.
